SEQUENCE LISTING (110 sequences)

SEQ ID NO: 1 - DNA, 59558 bp, Cricetulus griseus
  misc_feature 1..30020: note = CHO genome outside of ERV on 5' end
  misc_feature 30021..39247: note = ERV type-C, LTR to LTR
  misc_feature 39248..59558: note = CHO genome outside of ERV on 3' end
  source 1..59558: mol_type = genomic DNA
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttccatgtgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggagggactgacgggtccgaactcccgaccgcggctccaggagacgtcctggtagcgtttgaagccctcaggaagagggatttgtattttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatggggcaaactgtcaccactcctttgtccctaacactctcccactggaaagatgtacaggaatatgctcataaccaatctgttaatgtgcgtaaacgcaaatggattactctttgttcttcagaatggccgacctttgatgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcggaagcggcccctgacccttcaccaatggcttatcgactaagaggtcgtagggagcagcccgttccagattcaaccactctgcccctccgaactgggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggaagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccgggccaagcttccaccgccaagggtttctgacctcctgaacctagaagattagaggagtcggggccaggagcccccccctgagcccaggataacactgcaagtcggggggcatccggtcaccttcctagtcgatacaggggcacaacattccgttctgaatcggtcacccggacccctgagtcacaggactgcatgggtacagggagctacaggcggaaagcagtaccattggactacaaatcggcagctccagctcgcgaccgataaggttatgcattctttcctccatgtgccagactgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtacaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaagggaaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaatctggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagatagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactcccccctggaacttccgcacagagggcagaactcatagcactcacgcaggctctaaaattggcagaaggtaagaggctcaccgtgtatacagacagtcgttatgcctttgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttattggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatcccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccaacccaccgattcagccactgcatcagaatggaccgcacaccgcactcaaaatcctttaaagataagactctctcgtacaccctcctgttgattggttgtctgtttaccccccatgtagcaactaacccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctgggggaaggataggaacaagaagcaaggatttttatgtttgccccggccataaaccaactcatggctgcggggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagttgggccaaacatagtaatagcggaccagagatccccaacccattttcaagtccctaaaccccctaccgttcctaaagctatcactcctacaccaggtgctgtcaccttctcccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacaatggggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacggggattagatttattatttttacaggaaggggggctatgtgctgccctcaaggaagaatgctgcttttatgcagatcatacaggaatagttagggatagcatggccaaacttagggagaggctaaaacagaggcaacagctatttgagtctcaacaaggatggttcgagggatggttcgctaartccccctggttgactacccttatatccacgctcatgggacctctggttattctatttttgatcctcatatttggtccctgcattctgaacaaactgactcaattcatcagagaacgactatctgttgtacaggctttagtcttaactcaacaatatcatcagctaaagcaaatagatccagagtatctagagacctctgaatgaaagattccattcagttacaagagaaatgggggaatgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcacatgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctcccctcacctcctagcctcctggcaatactgcgtggccaggcacagcccaggcctcctccattttatttctatctcctgtgaagccactcccttggccccagccccagctggggaaaagagcacaggagtgagtctgaaactctccctagggatttcaagccagactgacttccagaaagccccgaaaggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacaccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacaggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctggggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtggaatatccatatttattgagcattgtgccctctctcttgggccttctctccttcctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaagggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctctcctcaataaggaaagcctgcttgatcctgtcacggacacacttggcacacaaggagccaccacagcccctgctgacgtgcttctttgtctagacagtctcataaggactttgggtctcacagcatgaacccttcaagtctgcctgggcacacaccacatggggattcaggtgctttcccagtcttcttggtataaaggtaaacaatcctgttgccagggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgcccccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacata

SEQ ID NO: 2 - DNA, 60000 bp, Cricetulus griseus
  misc_feature 1..59559: note = Locus of ERV integration, allele 2 (without the ERV integrated in allele 1, at position 30020)
  source 1..60000: mol_type = genomic DNA
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttccatgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctccccctcacctcctagcctcctggcaatactgcgtggccaggcacagcccaggcctcctccattttatttctatctcctgtgaagccactcccttggccccagccccagctggggaaaagagcacagggagtgagtctgaaactctccctagggatttcaagccagactgacttccagaaagccccgaaagggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacacccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacagggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctgggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtgggaatatccatatttattgagcattgtgccctctctcttgggccttctctccttccctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaaggggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctcttcctcaataaggaaagcctgcttgatcctgtcacggacacacttggcacacaaggagccaccacagcccctgctgacgtgcttctttgtctagacagtctcataaggactttgggtctcacagcatgaaccccttcaagtctgcctgggcacacaccacatggggattcaggtgctttcccagtcttcttggtataaaggtaaacaatcctgttgccaggggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgctcccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctcccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacatattaaataaataaaatcttaaaaaaagagaattagaaaagaaaaaaaagcttcatgtgtgtgtggaaacctccatgaggtagaatgggataaaagctcttgattctatgcttagtctggctagatcacctctcctgttctctggatctctctgacttgactgtagaggttggctctgtacaaggcagcactgtcagcagatcctgttggttcaaagaggtaggtgaagccaagagactacaggtgacagcagattgggagggcccagcacaggtgcagctcactggaagggtccagcacaggtgcagctcactggaagggcccagcacaagcgcagcaccttgggctatagactttgaccttgaccaaatcactttcctctctgcttccttatttctctccttacgaaattaaacaaataatcctcatttacctgcctcacaagttgtttagagggatcaaatattgctgttgctttgtgaactgtaagtagataagctatttagatgagatatatcatgattgacattcactcagccatgaagcacaggcttcctgacaggcactgctaggtgatggagaagaacaaatgagtctctccatctctcttttctcagcactaggattgaacccagggccttgcatacactaagcaagtgcactgccgccgagcttatctgtttttacctccttttaaaaatattgtttatgagctgggtggtggtggcacacttctttaatcccagcacttgagaggcagaggcagggggagctctgtgacttcgaggccagcctggtctgtagagtgagttccaagacagccagggctacaaaataaaaccctgtctcaaaaaacaaaaacaaaaacaaaaacaaaatcaacaaaatgtatgaatgcaaaaacaaaaggatactttagggtcgcaggtagattgggaacttcaaatgtgggccaaaagaaggctgtggccctgcttctgaaggatggtccagaccgaatctcagaaggacaaagtgtcggggctaagatgtagcttcgttgatagagttcatgcatgcctagcacgaaatcctgggttcagtctccagccctgaagaaactggatatggtggttcacgcccgtaacctcagcacttgggaggtagaggtagaaggaccaggagtttaaggaccgggcccagcctacgatacgtgagaacctgtcttgatgatgatgataatgggactggagagatggctcctgtgtaaagtgctgcataaacacgaggacctgagttctggtccccagcaccagcatgataccaggccatggcagaatatacctctagctccagtatcgagtagaggagacaggtggaccctggggcttgcctccccaccagcttagctgagacagcaaggtctaggttcaatgagagatcctgtctcaaaacaaaatagagataaatcaggaagacaccctggtgtcagctctggcctccacacgagcctgcaagcacacctgcccatacaacacacacacacacacacacacacacacacacacacacacacacacacacacacacacacacaccctggtgtcagctctggcctccacacgagcctgcaagcacacctgcccatacaacacacacacacacacacacacacacaaacacacacacacacacacacacacacacacacacctaaatttttttaattaatttaaaaagaagagacaatagcaataccaagatgcaatgacccagtggacatgcccctaactgtcctcccatcgtggagattgccaaggtgaggctgaagcagctaaagtctccatgaatgagaggaagctgtgagatgatggggagggtccccacaaagcctggagccttgtcagccttttttcctgcaccggaaaccacaggaaaccaggtcagtgtgtgtggctgctccctctgctccagagctcagctgtcagttttggggcttcctgagcaagctgtattatttagtttgtccttgaagctagagaccactttgacctggagtcgagaagagttggaaagccagctagctggggagccagagtgtacatggataacacaagaagtttatttgtgttatatgttggcctatttttttgcctaactttaaatgatgtaccaattgcttctttatcttcagcattcagtatagacaagtgccgaatagctgttaattctcaaaatggaagatgtgtaactgtctatgacagatccaagttctagaacatgctaataggcgtgccagatctgcagtgtttgaggagggttaagaaatgggtgcatttccacaacagggtgctggtccctttcctgttacctctccgccacaccaagagccccctgtgacgtgagagcagtaaggctttctaccaaacaggtggacaagtgaattagtgttcaccagtgccctggctcaggagaaggaatgtgacccagcctgctggttaggatagaggaggagcttcagggcagggtggatccagggcagggcttggtgagagcgcctgctggtactgtgtgctcctcacagctccagggccaggccctgctgccctgctgccctcctgcaaacaacaaagccatggtctctgtgccctgatcctgcagaagcttatggggagccccagactgacaacctggttcctgcctctcctcttactgctcttcagcctgtctatgtctgctgaggttggctgtccctacctgccacgctggaccagccactgtctactggcttcccatgtggtaaggtcctgctgctaggtgggggacacttgcttggcacagagtagaaactccccagtctcctgtctgccctttcctacctctgaaatccctgtgatctagtggcagcctctctgtgtccaccatgtggtcctgaaggttctgttgggctgaggtccttggaaggatgagaagaaaacagctggggtggatggacagtgggctgtgggtctcagaaccggatgatcggcacaaagagttcagggagcatgcacctcctgcagttcccttctaaccacacctctctcatttcttttccgcactgggctctgtacctccaggataagaattctgccggtgagtcctgtttcttgttctgcttcactccccatgctcccctgagttccaggtcccagtgagccatccatggccattttggttcccagcctggggagtagtctagtatcaatgggaaagtgcgggactagtgaagatttgcattgtaattctgcctcagacgctatgactgcttttcactctcccatctgaccctcggtctctgagcacaggagaagaggagaaatgattatgcgatgaggacagtgttgctctctaccaatggggaggataaaatgagcaccatggctaatctccaggcactctggacaagctggctcttcttatcaccggtacctcacttgaccaggaacaataatcatctttcatactttattgtgcattctgctttaccaagctttcccccatgccttatgtcacttggtccttatgacaaacccactttgagcagtctggcacctgcccaataacttaaccaagattgtacctgtaagctatatcagagcccagcccgaaatccagtttggagaaggtaggtgggaccccttaggccctaccatttttcttgttcctctttttgtttttgttttgagatagggcctcactatgtaataacaaagcctccataattatttatttacttattttggttcttcaagatagtgtttctctgtgtagtcctggctgacctggaactcactttgtagactaggctggccatgaactcacagagatccacctgcccctgcctcctgagtgctaggatcaaaggcatacgccatcaccacccagctgcctccatcttttaatccttccatctcagcctcccaaggaatagcttacagccatgtgttattgtgccgactttctatcatacacaggaagttctgttcaaactccctgccctaccctcctagccacgaagccttggcactggctctgctgtttgtgcctgcgtctgatgtcacggccttcaggtaagaaaagcagcctttgagccattcttagtgaaggcatgaatcagggacagttgaaaataactagctaggctgacacaagtgtcaattgtcatattgggacatatatttgtcttgttaacctcaagagggacttagagcatcctagaataacacatcatgacagacattcatgggaccactatatctttgtggaggtgccatctttcatctggcactaattcctaaaagccatgtgttctgtagacttgctattgttaccttccctctgtaaaatgggactgccactctgctgcttcattgcctcacttggtacatacggaacacacactttaaaaccagcagtcataggcggatggaaagatagcttagccatcaagagtactagatgctcttgcagaggacctaagttcaattcccagcacccatatcaggcagcatacaactgtaggtaactccaactctaaggaatctgatgtttctgatctcttaagcctcatttatatgtgccaatacatagatatacatacacatgcaagtaattaaacataaaataaaatcacaaaaagttagtttaaaattgttcaccattagctgagtgctagtggcgcacgcctttaatcccagcactcgggaggcagcggcaggaggatctctgtgattttgaggcccgcctgttctatagaatgagttctaggacaggctccaaaacaatacagagaaaccctgtctcaaataaataaataaataaataaataaataaataaataaacaaacaaactaagaaagaaatcatgctacaattaacactgcttttgtgggaggtgtgtgtgtttgtgtgtgtgtgcgtgtctgtcttttttctttgttttattttattttttaaatatcttatgttagaggtggagagatggctcagttgttgagacagttgctgcccttccagaggatctgtattagattcccagtaccctcatagtggctgtatacacaattgtctgtaactgtagttcagggaatccagtgccttcttttgacctcagcaggcaccaggcatgcgcatggtgtacattcatatgtgcaggtaaaaccttcatacacataaaataaaataaataaacctaaatatttaaaaaagaaatgaaatatcttttatttattgtttgacagtctcatacatgcagacaatctatcttggtcaaaaccacccacaattcctccctcccatgcacttggataactcccaatacatcactgctccacattcatgtctcttttaagatcttgtgtgtatgagtgctttgcctgcatgtatatatgtgtattacattcatgtagtactcatggaagcatcaaatccctgtagcactggagttatagatggttgggagccgccatgtgggtgctgggaaccaatcctgaatctaagaacatcaagtaggctgaactgctgatccatcttcacacacacacacacacacacacacacacacacacacacgcacgcacgcacgcacgcacgcacgcacgcacgcacacagagagccatcaaattttgagacagaggcttactgtgtagtcctggctcacctggaacttgcagagtagaccaggcaggtgtcaaaatcaagagatcagcctgcctctgcttctcaatacaagtataaaaccacgccttgctctgtttgttttgtaaattcacggagtccagctatgagtctgcctgctggaatgttgactgatctggttggcttgaccttgtgcagttcctgtgcaggtaaccacagctgcagtgggttcatgaacacgacagtcatgccatgtctagaagacttcaaaacactcttccacatcttctggctctcacattctttctgccctgtcttctgcatttgctgaaccttgaaggggcttaattctgttttttactgaatcataatttttgccaccaaagaagctcaatatcattgctacccttgatttctataaacattctacccaacattgggccacatctccagataggacaggggccaagaatgcccaggtaagaagtgtgtttccctcccacaggccttcagtggaactggtttcctctcttggtgaagaaatctaaaagtcctcctaagtctgaattctactggaggcgtagaaggccagcatcgtcccaggtaccctatcatttacaacactatggccctcagcccctttctttcttgccccttattttgttcttcagcaacaagagggagtttgaaaatgtggagattacgcactaagggaaactgaggccaaaggaatttgaacaacttgcctgttaaattaatttgtctctcagggttcagaggcagagttagaaagcacacttcctggatcctggcagacttcctggatcctggctcacatccactttgttctctgcttgcagaggaagctgctaagcagcctttccctgtctcaggaagaccatagcatttccaaccccttcccagccatctcccatggagggccacactgcaaaaggacccagcctttggacaccggaggagtagaacattttcctagagcaggttcacagaagcgtggaggtaggtgcgaggagcaacaagctggtggtctcagccttgcctgagccagtctctgagtcgtccttctccctaggacctgaattctcctttgatttgctgcctgaggcacaggctattcgggtgactattcctccaggcccagaggctagtgtgcgtctttgttaccagtgggcactggaatgtgaagacatgagcagtccttttgatacccaggtatggagttccatgccttgcaaagagacagattacacaatttctcagcaaatgggatggggtgggggggttggggcccagacaaggaaagtaattgtccaaaacttccttacaaaatgagataagtagcattaaaaacttgttgggggcgggggaggactagatagaaaagatggtgggctatgttacttgagaagagagaccagaaagaaatgagaatggtatgtattgccagggagaggactcttggtgtgattcgggtcaaattaagagactttgctaagggctggggacatacccgttggtagaatgtttacctagcatgcttgatctctggcactgcacagatggagtgggaggtggaagcaggagaatcagaagttcaaagtcttccttagatatataatgagtctgaggtctgtttggagtacgtgagtcttaagagaaaaaaaaaaaaaaagctttgttatttcctcaggaggttgttccctgaaaatttgacaaaactgtcatatgtgtcttggtacttttccctttcataccttttgtctagccctcagaaatgatattccccagtctcacaatccaggtcttgacagcctactttaactgccactcctcacagatgccattgtatgtagctgggcatcacatgcagagactggagtccttcagcttgcttcatgtctacaatggctataccttctaacaataccagcctttagtagaattgatgcttctaatactgcatggcatacagctggaactcaagaaatgtatcgcctcttccctaaccctactgcttgtagagtacctcaagaaaatggccaggatatagaacctatgagagggagaaataagttccatggagtaggaaagtaaggtatattgtccaaggtcccggcttcatcctgccttgtccatagtgtccatgcttttccttaggtgtctctttttctttccctttgtggttttatcaacagaaaattgtgtccgggggccacactgtagacctgccttatgaattcctcctgccctgcatgtgtatagaggtgagcacagggaaatgtggattagccatggctccttgtccaaccgcatctccagtaagtcaacttagaaaggactcgggaaggatggagatgacttggtgatagagcaggccgtacacactaaggagagactccaaaaaaaccgggctactgggatgcttaggaagctggtgctggctaggctatgagccacaccagaggtggaagggtcctaggcaattccacctcagccctgcctggatgctgtcttctctggtccagatttggcttcagctcacagacacagagtagacagccatcatcgctgtgttcttgagagccaaggttgcatatccctctcaaagtcatgttctttccaaccctccatttctccttgtaagtctgaagataaagatgttttggacatttttagggggaagacaggtactaaaagaagggtatggacagagacctgggaaacaagaagctgacagaggagacaaccagtggactggactcgtagggagtccagagtgaggagtcaacacagggtactgggtacttgagaaagtcaacaatggagctcagagagcctaaaattgagtgatgcaacctcgggaattctctgctgctgggaacagttctttggagacaggaggggtcatcccctctcatttgccactccctcctatgctctggaaccccaacacttacacacactccacagtgctgcccattattgtctgacagctcaagctgtttgatgacagtaagccagagccagaatgaagatgttaggaaagccatttctaacaaacactctctatgctccccaggcctcctacctgcaagaggacaccgtgagatgcaaaaagtgtcccttccagagctggcctgaagcctgtgagtgttgtttgtattaatcccgatgcacattcatactccccctgctgtgtgtgtgtgtgtgtgtgtgtgtgtgtgtgtgtgtgtgtgtgtgtgtgtgatggaggaagaccccagatggcaggaggacattttcttcaggagccaggggaagatgccacagctgtcagccaacagatgagctcctgagtgctgaacattatgaatggaaaaggatcgcccagcgagtgtgtgagagggccaactgtagacctaggctcacttctcagttctgccataggttatccccatagcccctcccctttccctcatctgtaaaatcccaaactgtttttgaggcttgaatgaaatattttaggggaaagtggctggtgttgtctggagtgctgtatgtagaatggtgaccttgaggagagaggacacttagacacaagtaacaaagtctaacgtacataggcagggttatcatcaagggtatcagcccaggacaggagaagcccagagacactgttgtgagcagagagggtgtcacaggatggcacttgactgagctcaggaactggacaggaattagaaagaagaagcagggggaagacagactctgtgaggagcctacacagattctggatacaggcgacagtaagggctgttcagtgtggaggcctggatggaaaggtaaagccaaggaagctgggtagaaagggagggagcgtggagtgcttgcctgttgaggacctgtgaagcctggaattgtgctggaacattggtattttctcctggtgtcctggggtggggtggggagatggagttacagctgggctatggcctcactcttgcacctgccacctgcccagatggctcggacttctggaagtcagtacacttcactgactacagccagaacaaccagatggtcatggctgtgacact

SEQ ID NO: 3 - DNA, 9227 bp, Cricetulus griseus
  misc_feature 1..9227: note = expressed and VP-generating type-C group 1 ERV
  misc_feature 1334..1336: note = Myr motif
  misc_feature 1851..1868: note = PPYP motif
  source 1..9227: mol_type = genomic DNA
tgtgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgtctccaataaagcctcttgtttttacatccagttcgtggcctcgctgattcctgggtgtgtgggtctccctctacgaaagtgcctcttcggggtctttcatttgggggctcgtccgggattgagacccgcccagggaccaccgacccacgtctgggaggtaagtgttgtgcggatccgctgttttgtcttgtctggtctgagtctgtcttgtgaattgcgcttgcgtttgtagtatacagctgtgtacatttgtaggcggatccgaggagggactgacgggtccgaactcccgaccgcggctccaggagacgtcctggtagcgtttgaagccctcaggaagagggatttgtattttgaacttgggaagccctcagggtgagagatttgtactttgaacttagatctatgactggacattttcccagtctctttggagaaggccctcggcttgagggatttgcaatctttactggggacgaggaaggagggcccccttcctcgactctctctcaattccttctgtcgactctctgtcgaaaccgcgctgcgaaagtctgttctgtgttattcggtctttgtcttgtagctgtcatttgtgccctcctaagcctagaaactatggggcaaactgtcaccactcctttgtccctaacactctcccactggaaagatgtacaggaatatgctcataaccaatctgttaatgtgcgtaaacgcaaatggattactctttgttcttcagaatggccgacctttgatgtaggctggccgcgagatggtacctttaacccccagactatattccagataaaagagaagattatggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcggaagcggcccctgacccttcaccaatggcttatcgactaagaggtcgtagggagcagcccgttccagattcaaccactctgcccctccgaactgggctgaacggccaacctcagtattggccattctcagcatcggacctctataactggaaaaataataatccttctttttctgcagaccccgtgaggctgacatctctcatagagtcggtactcacgactcaccaacccacctgggatgattgtcagcagcttttgcaggtccttttaacctcggaagagaaacagcgcgtgctactagaagcacgaaaaaatgtcccaggagtaaacgggcagcccacccagctacccaatgaaattgatgcggcttgccctcttgaaagacctgaatgggattttaccaccgaagcaggtaggacccacctgcgtctctatcgccagttgctggtagcgggactccggggggcaggacgcagacccactaatttggcccaggtgaagcagataatacagggtgcggaggaatcacctgccgcttttctagagagattgaaagaagcgtacaggatgtatactccctataatccggaagatccaggtcaggccaccaacgtttctatgtcctttatttggcaatcagccccggacataagaaacaagcttcaaaggctagaaaatctacagggatatacactccaagatttgttgaaggaagcagaacgtatttttaataagagggaaacacagacagaaagagaagaacgttggaggaaggaaactcaggagagagaggaaagactaagacaggaagctgaggaaaaagaggttgcgagagaccgtaagcggaataaagaaatgagcaggttattggccacagtagtgacaggccagagacagaatagacagagggatgacagaagggggccccacctggacagggaccaatgtgcttactgtaaagaaaaaggacattgggcaagagaatgccctaagaacccccgggccaagcttccaccgccaagggtttctgacctcctgaacctagaagattagaggagtcggggccaggagcccccccctgagcccaggataacactgcaagtcggggggcatccggtcaccttcctagtcgatacaggggcacaacattccgttctgaatcggtcacccggacccctgagtcacaggactgcatgggtacagggagctacaggcggaaagcagtaccattggactacaaatcggcagctccagctcgcgaccgataaggttatgcattctttcctccatgtgccagactgcccctaccccttactaggacgggacctattgaccaaattaaaagctcaaatacactttgagaggtcagaagtcaaagtcacagggccagagggaattccccttaccatcttgacaatgtccatagaagatgaatatagactccatgaaaagaggactaattcgaacaatcaggaaacccttgatcactggcttgcggaatttccccaagcctgggctgaaacaggaggaatgggccttgccattaaccaggccccaattatagtaaccttaaaagctgccatccttcctgcatccgtcagacagtatccaatgcctaaagaagcccgcgaaggaattcggccacatattaaaaggttacttgaacaagggattctggtgccctgtaaatctccttggaatacacccttgctacccgttaggaagccgggaactaatgactaccggccagtacaggacctgagagaagtcaataaaaggatagaggacatacaccctactgtccccaacccttacaatttgctgagtggattgccacctaactatacctggtacacagtcttagatcttaaagacgctttcttctgcctccgcctgcatcccaccagccagcctatatttgcctttgaatggcaggacgcggcccttggaatctctgggcagctgacttggactaggctacctcaagggtttaagaacagccctaccctttttgatgaagctttacatcaggacctggcagaattccgggttaggtaccccgctctaatcctcttacaatatgtagatgacattctcctggcagccaaaaccaaagggaaatgcaaggaaggcactcaagccctcctccagactcttgggagcctagggtaccgggcatccgccaagaaggcccagatatgtcagaaacaggtgacctatttaggatacaagataaaggatggacgtcgatggctaacggaagcccgtatgcgagccatcttagacattcccaccccacaaaatccccgccaactgagagaattcttgggaacggcaggcttctgccgcctatggatccctgggtttgccgaaatggcggctcccctctaccccctcactcggccaggggttgcttttaaatgggaagagccccaaaagaaagccttcaccgacatcaaaaaggctctccttgaatcaccagccctgggtctaccggacttagctaagccatttgaactttttatagatgagaaggagggctatgctaagggagtcctcacccaaaatctggggccttggagaaggcccactgcatacctctccaagaaattggatcctgtggcatcgggatggccaccctgccttcgaatgattgctgctatagccctgctggtaaaagattctcacaagctaaccttggggcagcctttgaccatacatgcccctcatgcagtagaggcagtcatcagacagcctccagatagatggcttactaatgcccgaatgactcattaccagactatgctgttagacaaagaccgggtccacttcgggcctttggtgactctgaacccagccaccctgctccccctccctggggagcccgaggctcatgattgcttacaggtattggccgaggcccatggagcgagatccgacctgactgaccagcctctacctagcccggaccacatctggttcacggatggaagcagctttttgcatcaaggagaacgaaaggcgggcgcggcagtcaccacagagaatcaggtcgtctgggcccaggcactcccccctggaacttccgcacagagggcagaactcatagcactcacgcaggctctaaaattggcagaaggtaagaggctcaccgtgtatacagacagtcgttatgcctttgccactgcccatatacatggagaaatttacagacggagggggctgcttacctccgaagggaaagacattaaaaataaggaggaaatcctcgctctcttaagggctcttcatctgcccgctgccttaagtatcatacattgccctggacaccaaaaaggggattctttcgaagcaaggggcaatcgaagggcagacttggctgcccgagaggcggccctgaccacagacaccactaacctcctggctctagagcccaccaacgaccatcccttcccctcatgggactatgaacaaagagacatccaaaccctagagaaattgggagccgcaaaggaaccaaacggggattggacttatgaaggaaagactgtcatcccctaccgggtaaccaagtacctagtgacatttttacataagatgacacatctgagctccaagaagatgcgggagctcctcgaacgagaagaggaattcaatttccttttggggaagaacgatattctaaaacaggtaactgagcaatgtgatgcgtgcgcccgagtcaacgcatccagactgaagcttcctcccgggaaccgggtcagaggctaccggcccggaacacattgggagatagatttcactgagattaaaccaggaaaatatggatacaagtatctattaatttttgtagacaccttttcaggatgggttgaagccttccctactaaacatgaaacagccaagatcgttactaagaaattgcttgaagaaatctttccccgttatgggatgcctcaggtattgggaacagacaatgggcccgccttcgtctcccaggtaagtcagtcagtggccaccttattggggattgattggaaattacattgtgcttatagaccccaaagttcaggacaggtagaaaggatgaatagaacaatcaaggagactttaacaaaattgtcgcttgcaactggcactagagactgggtcctcctactccccctagcactctaccgcgctcgtaatacccctggaccacatgggctcacaccctttgagatcctgtatggagtacctactcctatcattaactttcttgatcaagatgtctcagattttgctaactccccttctctccaagctcatttacaggccctccaactagtacaacgggaggtctggaaaccccttgctcaagcttataaagaccagagggaccatcccaccatcccccattcctaccagatcggggacactgtttgggtccggcgtcaccaggccaagaaccttgaaccccgctggaagggaccctacatcgttttgcttaccactcccaccgcactcaaggtagacggcattgcagcttggatacatgcttcacatgtaaagccagcccaacccaccgattcagccactgcatcagaatggaccgcacaccgcactcaaaatcctttaaagataagactctctcgtacaccctcctgttgattggttgtctgtttaccccccatgtagcaactaacccccacagggtttataatatcacctggaaaatagccaatctagggaccggggaaatagccaacctcagcacttatatagggactctacatgatgggttccctcctctctatgtcgacctatgtgacttagtagggtctgattgggatccctctgaccaggaaccattcccagggtacggatgccaccaccctgggggaaggataggaacaagaagcaaggatttttatgtttgccccggccataaaccaactcatggctgcggggggccgcaggaagggtactgtgcaagatggggatgtgaaaccacaggggaggcttactggaaaccctcttcctcttgggatttcatcactctcaaacggagggagatcccagggtacgcagggaaaggaccatggagatgtgggcaaagagcctgcggaccttgttatgatagtgccggagggggaggttttcaaggcgccacccccggaggaaaatgcaaccctctcatcctaaggttcacagatgctggaaaaagaactacttgggatagtcctaaggtctggggactcaggctgcaccgagcagggaaagatccggtgactttattctccctgtacagacaaattactcccctaagccaacaatcagttgggccaaacatagtaatagcggaccagagatccccaacccattttcaagtccctaaaccccctaccgttcctaaagctatcactcctacaccaggtgctgtcaccttctcccccaccccagatgccctaaacatcgagataaccagagaccctccaggtaccagagatagattattacaattaatccaaggagtttaccaagccttaaatttttcagaccccaacaagactcaggaatgctggttatgcctagtttcccggcccccatattatgaaggcgtggcaatactgggcaactactccaaccagacctcagcacctaccagttgcggagctgctatgcagcacaagctcacaatatctgaggtctcaggaaaggggctatgcataggcaggattccttcctcacatcaagaattatgtaaccaagtagagccattatctcaggacagccgataccttgttgccccttatggaacttattgggcttgcagtactgggttgactccctgtgtctctaccactgttctcaacaccaccattgacttttgtatattgatagaactttggcccaaagtcacataccaccaacctgaatatgtttacagcgtactagagaaatcaacccgatataagagggagccaatatcctttaccgtggccctattattaggaggaataacaatggggggcatagcagccggcatagggaccggaaccgttgccctacagggaattaatcattttaagcttctacaacaagccatgcacacggatatccaggtcctagaagagtcagtcagtgcactcgagaaatccttaacatcactctctgaggtggtcctgcaaaacagacggggattagatttattatttttacaggaaggggggctatgtgctgccctcaaggaagaatgctgcttttatgcagatcatacaggaatagttagggatagcatggccaaacttagggagaggctaaaacagaggcaacagctatttgagtctcaacaaggatggttcgagggatggttcgctaartccccctggttgactacccttatatccacgctcatgggacctctggttattctatttttgatcctcatatttggtccctgcattctgaacaaactgactcaattcatcagagaacgactatctgttgtacaggctttagtcttaactcaacaatatcatcagctaaagcaaatagatccagagtatctagagacctctgaatgaaagattccattcagttacaagagaaatgggggaatgaaagacccctctcccttagccctttctttctcaagtttgtctcctcttcctcctgtcggcggcttccccgatccccacccccggtggcctttccccgcccggcccgagaacaagcaccgggtggggccggcccgagaacaagcaccgggtggggccggcccgagaatgagcaccaggtgggccagcccgagaacgagcaccgggtgggctggcacgagggcgagcaccaggtgggtcagcacgaggacaaacaccgagtgagccggcctggagctctgcccctgagcccccgccccgcccgaagagaaacactccgtcccaaggtctccgcccccaaggtcagccatcaggaaaagggggggaattgagtctgctgtaccagacaccagaccttgagaatatgctgatctggaatggctctgtgtctcatttgaaccatccaatggaaatgattctgtatttcgcctcatttgaaagactctgtgtttcacctcatttgaataactctgtactttgcctcatttgaataaccctgtatagcgcctcatatacattgaccaatgggaatagctctgtataatgcctcattagaattatccaatagaatccttgctcctagcttgcgccttttttcctatataaggaccccttttcccttggctcggggcgcttagccacacagaagctaagtcgccccaggtacctgcgt

SEQ ID NO: 4 - DNA, 30019 bp, Cricetulus griseus
  misc_feature 1..30019: note = 5' of SEQ ID NO: 1
  source 1..30019: mol_type = genomic DNA
tggttctatcgagacagctacatccgcttctccacacagcccttctccctgaagaacctggacaagtgagctttccctccacctcccttgactgcccaggctagtgaggaggtcagcagacaaacagaacagtgggctctgcgggcagggaaggcaggcttttcgccgccactcatcctctgccctgaaggagcttgccaagagggtgcccttgggttaccgaaatgagcaaaacaagaactcctgctaatcaagggcttacatcctagcaatgagcttgagtaatcatggctaacatgctagaaggtgatatgtgttaggaagaaaatgtagatctgggtaatggcagattaagagaaacttgagaggggaaaggcaacttgcagtttgctcttgggatgagaggaaaccttgaggagacctgggttcaaattctatctctgcagcatccagattgtgttcctctagagacatgaaaagagacttaagcctcagtttcagttagagcccagtggtcccatctgtaatcccagcacttgggagtctgagtctgaaggattttgaggtcattctgggctatataatgagacccccactcaagacaaaacaagccaggtgtggtagctcctaactgtaatcctagaacttggctaaggcaggagccaaaatcaagccaggtacggtattggaggcccgtgatcccagtgcttagaaggtagaggcaggacaatcagttcactgtcaccctcagctacatgccaagttttatctaacctgggcaacatgagaccatttcatagaaaaagaccagcctccgtttccacatctgaagactggagcagcaatacactgtagctgcttatcatcaagtacttcacatgtgataagttgttcctgcttggttataccacagccacttctcaatggctcacaatgaagtgagttattgccattttaaaaagagatgagaggggcaaaggttaaatgacttgcctgagatctcatagttgatcatcggtgaagctgggatttgaccatccagggtcatcgtccccctgccacacataggaatggtgaactgaaactaggcccaaagcacttgatacttactgtggcctgcaggcaacactggagcattctgagaaatgactagccctggccgtgggtgaatagggagtggggcagggagtgggggtggggtggaggggtgcagcttctgtgcttctgtcctgttgatcttccagcaggggcccctctcagttgagatcccctagacggctgaccacagggttctgcctgcagccctgaggttgggctccagggctgagcctgtgtctctgatccactctagctctgtgcacctatgtaacaattccatccagagatacctggagacctcctgtcaccggcaccggatgctgccctcggacaacatgtggtccagccagaggtttcaggcccacctgcaggaaatgggtgccccaaatgcctggtctagtgtcattgtacccggcatgaaggctgctgtgatccatgccctgcagacctcccaagacactgtgcagtgccgaaaggccagctttgagctctatggggccgactttgtgtttggggaagacttccggccctggttgattgagatcaatgccagccccaccatggcaccttccacagctgtaactgcccgcctgtgtgctggtgtgcaagcagataccctccgtgtggtcattgaccggcgactggaccgtacctgtgacacgggagcctttgagctcatctataagcaggtgagatgtcccagcacctcccacaggcaaccctacagcaaagccctggctggggtctgctgtgagacagagttcaagactgactctacacacggggcatcttaacacagacacgtcccactggcctgtctcctcatctgtggaagatactgtcctttgagagccattaatgccatgagtttgtagtcataggtagtattttcagaggccctgggacctgctcagtttccatggtaattccaagcctctaggtagtaaccttattctctcatctgtaaagtaagactgtacctggctcctcctttgtgtgctgtgagaatgggtggtttgatcttcacacaagggtttgctaaagtaccaagctggaggacatagaggatatgaggccatgaacctcaaggcctgcctatcaagagttaattattagtgtctatcattgttataacgattattatgttactcatcttcttccaaaaacagatttgatcttgcttatttataataagtatagaattgcttggggtttttgtttttgtttttgtgggttttgttgagatagggtttttctgtatagctttggatcctgttctggaacttgctctgtagaccaggctggccttgaactcacctttgactgcctctgcctcccgagtgctgggactaaaggtgtgcaccaccattgcccgacctagaattgttttttattcagccaaaaaacattaccttgccctcgtggtctaattctctctagaaacacccttaggagcacaccaggggcatgccctatagaaatcctaggttttctcagtccagtctagttgacaactgatattagccaccacagctggacatggggctccaccttccacctcccagtatttggaaggctgagacaggggatcacttttagttcaagggaagcttgggttacacagtgagttccaggctagcctgggcttcacagtgagaccctacttaaaaacatcatacaaacaaacaaacaaacaaacctttaaacgtatgtttttaagggtttctgaattctgaggacagtttttaagatctttgagctaagattcaccaaggtctaggtttgctacaggaagggaaaacactgatcacctgacctgggtggcctcatgtttcctacaggtcctgatcaccttagaataatgtgctggcaaagggttccgtctttgttagggatgccatcactaggtgtccagggtggaatccaggcctgcgtttttagcatgtgcagttctactgagctacacctccagcctaaaaaatgtaaaggaggagatgcatgcaggtgtgcatacacctgtaatccctgggcaacagaagcaagaggactgtcacaggttcaccaccacctggttacagggtttataataaggtcctgtcacaaacaatgtagtcttggaagagaggagaggaaatggggaagaggaagtaatttgcagtttaaaatagagcaaaattgccgggcgttggtggtgcacacctttaatcccagcactcgggaggcagaggcaggcgcatctctgtgaattcgagactagcatggtctacaagagctagtttcaggacagcctccagagctacagagacaccctgtctcaaaaaaaaaaaaaaaaaaaaaaaaaaagcgaaatttgtatggacatggcaacacgtgcctgtaatcccaacacttagggggctgaggttggaggatcaggagttcaaagttatccttggctgtatgtgagcttgaagccaacctgggcctacagcactgtctgttccacaatctgtctttatctgtttgtctccttatgttgttcagctcggtctgttctctgaatgtttgtctcagaacaaacaaaattgaatagggctgggcatacgcacttttcaaatgtcctttgtcccccaggtatctttcattgctgatttggttttgagcttgagggtcagtgtacaacaaggtggttacaatggtggctttgtctgtctctgatctcctttatctaggacagtgccactgctgtatccctggcaccctggtcctttgcaggccaggcaggccagcctgcaccaccgccccacactgttttatctgtttttctccttatgttgttcagatcggtctgttctctgaatgccctgtaaactagaagataggctaacaagctgatggggttcagggttgagatttttggcaaaaaacactcatgtgatgctaggtacctcatgtgactgtcacaaggcacaaccaggaatctctagttcccaatgccgaatttgaccttagactaaggtggctgccaccagagccacatcctcccctttgtagcttttttcatttttctttacattatttattgtgtttgagtttgtacatgtgtgtggtcacacatgccacagcacacatgtgggagtcagagggcaacttatgggagttggttctctcctcccatcccatgggtcctggggcttgaactcagcaagtaccttataagctatctcaatactgtttgcctccaaaatgtattactttgtgtagctgtcccttttctgttgctaataacagaatactacagacagtgtaagttacaaagaaaataggctcaattgtatccatcttttgctgcccatggcatgaaaacacttgcatcccaacacaggccagaggtcgctttagtgtgaagcaggattgagtgcatgtctgcatggctaagactctctacttctctggtttcctgatccctagggctctgggactctagatcctctggaccccttgataatagagggagcttcctgtttctcagaagtgccttttgaccatatatcccagaaactgattccatccatctctgccgtgtgtcactagtcactagatggcgtcactgtcatctctcactagtgtctgagatggccttgtcttctctcctgcccacagcctgctgtggaggtgccccagtacgtggggatccggctcatggtggagggctctaccatcaagaagcccatagcagcttgtcatcggcggacagcggtccgctcatcactccctcatctgctggcccagcaaggctgtggggaaggcaaggactcaggaccccctatccacaggtcagcttctaggaaagatgctggggccaggagcctgggacacactgagaagccagactctgcggccaccacctcagtccccggaaaggggaagaaaggcaaggcaaaaagtgccacagccctggtctgcatcaccctgcagaaatgggagtcccacaacaccagggtgggccccaccttcaacaggttaatgtgtctgaaacagcctgaggcctggggtagtaccatgtcccccaaaccccgcagtgttcccaaggccatttctgcctgctctccaagccctccccaagcatctgggcttgccctcctgccaaaaggccaccagtgatagcaagtatgaaccaaatatctttaaatacataaccaaatgagtattacaaagtagtcaccctgccaggcagttagaccaaaggctcggtcctagagtgcgcgcccagagtccagacccatgctgctgctctagccagcctttgccctcacctttctctggagaaaggtgctgccaccatgcccttccccattcctaaccagccccctcagccctcataacgccctagtgaggtaggtgctattgtccccattttccagccgaggtagcagcaagtttaaggacgttgcccgaggttgcacagctcagaaggggcagagctgggatgcagacccaggtctgttggtctcccaaccctgtgttcttcccactgcctctggaggaggagctgggaggggctccatctgcccttaccttgtatcccccacctttacacatgtactgtggaacaattggtcaggctggggcctcacccagatcctcacagcttcccttctcccacagccccgtcctacctccctagtctccattccaaggcctggctgccttcttcccatgtgctccgaccccagggccgggtcctcagactaccgaatggccaactggtgggctctaaggctctgtcaaccacaggcaaggccttgatgactctacctactgccaaggttctgatgtccttcccacctcaccctgatctcaagctggcacccagcatgctgaagccaggaaaggtgggcctcgagctgtgcctcacaccctggcgggtagtgctgagcagtgggatcggggctgaagggcacgaacagagggcagcgctcggaccatacagcgccccagggaagggcttgtcttctccagaaccctgttccaagacagaggcctgatcatatctctttccctcccctccttgcaccgaggctgctattcccctgcaccttcgaggcccccactttggaagtgcctcgaggcctctgccctttgaagttggacctcttcctagcacccacaggaaagtcacggccaaaggcaagttcaaggccatactctgcgacaaagccagggctgaggcataccccaagaagaggctgagcctccccaaacccttgacccttattctgacatgccggacactgagaaccatgggggataggaggctagagaaacccctgctctgatctctactgccccatcctggatccagcatcaaattaaaaaaagcaattaaagttctctggacttggcttgaataatgtgcggctaggctcataaaagagttgaccagcagggcctccatcagcaagggccacagtccccacccagcgacagacattggctttctctgcagggagacggatgggtggggaaagagccttcactatacagatgatgacactgagacatggcttgcctgagaccacagcaggcggaaagtcagccatcaggagtgccccttcccaaagacaagctgggctggcagaagagcttctgatggtcacagaacattatgacaagagacgggctttccctaaacctcagcctttctccggagagtatgtccctcagtgaggggtcggtcaagacacctcaaactgcaaaacccaagaaacaggtcaagtgtggcatttcctacctgtagagcctcagctgctggtcctccaaaagcggtatctagctgtagcgtgtgaatgcttcctgagtggggcagggtggagaggagagtgcggtgttgaaatccaatgacctgtgtctcccaaagtcagaagagctcatgcctgcacagtggtgtgtgtctgtcctcccaggacttgggaggcagaggcaggtgcatctctgaattccagtccagccagggatataccaagaggcccagcctcaaaagcaaacaaacctcctcgtgaccaggagtacagcagatgccacctccagacctggccactgttcacttgagcagagagcacagtccctggtcaacacttgctttctcagcagatcctcaaaagcagacttgtgaggtaggacttttattattttaagtccagagagcggtcacctgcccaacgccacacagtaagtgagtggtttaactgggatttatcttaggttggtaggactaaggagtcaaagaccttgaccgtactcagcaccaagcccactgtccttgagctgggtcacggcccttctttctattttccaattgggggttaagcactgtctatcggtgagagagccgcaggcactgcagggtccgagagaagtacgagggtaggggtgccacaagttcactaggggggtccctggatctggtgcctgggaggaggaggcggtgcaagtgcaggtgcaagggcatctgggccacctgggaaggacgcaggcgaaggcgtctgaggagagcttcgtccagcacctggagtgggaaagacagcagcccacctgagttccagccagaggagcccctggctctgatggacctctctggtctgcaactgccatcattttctcaacaggcaggcagggatttctctccacacagagctaagttacgtttcagctccttttgtgtttagtgaagccatgtgattaagccactcaccaatgggatgtgaggaaaacacagacctagccctcaaagccccgattcacaaaaacatccagtctccccttatggccaggaagcaatagcccttttctccaagtggctacccagagtcaggtccactcctactgtatgcccatttgccagacttaatccaagaagaaacaatggacttcagggcctgtcagaggtcagctccccactccttgagctaacagacagaaggaagcctgaggaagtcacagcaccacagaggcaagggtggccccagaggccaagcctgttcccccaattccctaaatacagagcaggcatgtgggcctgaagacctacctgtctctggagcttggtggtcttggctggccacagaaagcgctggcccagaacggtgcccactcgaggagcataggtgtggtccccaagcactgggcagagctgtagagccatgtgcacctgaagctgactggggaacactgaaagatggggggaggcagttgctcttcttgatcatcaaggactgtccccattcccagagcctgtgcatctatgaggtgacctctgcctccacagagggccatgagcagatcaggacaatgacagctggcaaccttcccagcctgggggcattgaacccaacagaactgaagctcctgggaataagtatgtgggttgggtgctaaactgtgggtgtgcacctaaaactctgcctcctgctgagcccactctcagagtccccagatgctcttttttttttgttttgttttttgttttttcaggcggggtttctctgtggctttggaggctgtcctggaactagctcttgtagaccaggctggtctcgaactcagagagaggtgcctgcctctgcctccccagtgctgggattaaaggcatgccccaccaccgcccggccaccagatgctctttggatgttgcaaaacaacatcttcctcataggcttcatttcccctgcagagtactgttcagccctacctgtcagtggctgcagctggaccagagcacagccacagcctgtggccatcacatggaaatggctgagggtcctcttgacaccttctaggatgtcctttcgagatggggatgtcacggggatggcctaggataccagtcaagaatgacttagcacacacgtaggagcccaattgagccacccctgctgccggctgacttaggacagggcctcagaacaggcagcagcaacttgcctgttagaggacaggggaatagccaccccatgcacatagtgcagactcctctgtaaggcaaaacctgaaaggactcctagtggcttctgactcacaagatcaatgccatccatgcgttccagcttcagggccacgtggatagtcccctcagaaggctcagggatgccatcagtgatgccactgagaaagagaaaggaagtctcagagcagccagctgggaccttccttggccctgggagtcaccccgcatctctcaccagtaggtggctgtgggcctctgtgttctccttgagtgaacgaagaacttctgcaagcggcttgctgtctgggggcagctggagagaagcacaagcccagacgcctccctgaaagaagaggacaagtcacataaagcctcttggttggaaaaaatgagggccagattggctgctcctgcagaggactagcactcggcacccacatagtgggtcccaactgtaattccacttccaagagagctgatgccctcttctggcctctggggcaccgggcatgcactgtggtacacagacacacatgcaggctggcctccgggggcacaaggcatgtgtggtgcacggacatacatgcaggctaaacacacatttaaaaacaaatctttggtcttttttaaaggagaccctcccaccaaggggctggagaaatgaatcagtggttaagagcactagctgctcttccagagtacctgggttcagcacccacatggcagctcacaactgtagctccagttccaggggatctgagaccctcacacagacacacatgcaggtaaaacaccaatgcactatatatatatatatatatagatagatagatagatagatagatagagagagagagagagagaccaccttcccacctccccaaatgaacaccaggactacagtccagacctaagaaacaaatcctggccaggcagtggtggtgcacgcctttaatcccagcacttgggaggcagaggcaggcgcatctctgtgagttcgagaccaacttggtctacaagagctagttccagggcagcctccaaagctacagagaaaccctgtctcaaaaaaaaaaaaaaaaaaaaaaaaaaaaaaaaaaaaaaaaaaaaaaggggggggaaataaacaaatcccaagaccttttttctggcccccagagaactcagacaaatgccctaagagttactacacatgccaggcacctgaataggactcttatatacagtgggtgtaaaataaaggcccaccacactcctcacttactttccaggtgctcgcacaacctggagctcccggtgtttgagccccagggcctggctcagctgtggcagcacagaaagcaaggtcagctccccaggtctccctgagaagtaagagggagaaaacaagttgccagagcaccatccaacgaactcctccccaccagcctttccctccttgtccctgcccatacctgtcacaggcagaccctgtggcttgttcagtgtcaccagaggtcctgaaatattcggtagatgtcagcaaaagcagggcagcctgtgtgcatgcagcctctggttataagctcccaagcccactccaaactgaacttcctgctccccgcacccagggcctttgcacctgctgccctgagctttcattaatattgggtcccttatcacacacctccctataatgtgtgcccctcctacagtccttgactgtttggtctgagaccagctcaaacaggccggcctcaaactcaactataggtaagtctggtcttgaactcttgatcctccttgcctctgtctcccaagttctgggattacaggtgtggtcaccactcccagaaacagcagagattcactattcctcacatcagaacgcttgctctctcaaggcaggagccagccatatttatcccagtactgtgtagcctcctcattgctacaatgcactgaagttcagcaaagttgtcattcctaaaggcacacagcatgctgcttacagaccccaggaaactaacttctgctcccgagggtcttagaatgcgaggcggtgtgaaggtttcctatgcctggcggagggaatgtgacggctacaggggtccgactccagggggtcggaggctccctcaccttgctgatccaccacagctgccctcagcacctcagccagctcctccgggccgaggttctctgttcgcagaagcccggggaagggctggtcctccactgcgtccttgcacttgctggaaccttgaggttgaggccgacccctgagaaaaacgacagaagccggtacctccaacgcccacgctgcccagtgcagccttttcgtacacaaccctcagaacccccggctcggctgacagttgtggtccacaggcccccaagactacagaaggggcatcagacgctcggagcgccaaccatctcgtgtcgggtcacacaacgcggctgggattagaacccatgtcctgatctgagtgcccgacctgccctccccttgtcctagcctccctcctacacggaagtgttgagttataatcaccgggcctcggtgccgaagcctgcatctctcggccgtgcgccagccgccagccggggccgccaggtgccagacacacaacgccaaacgcggaagacgcccatcgccgcagcaagcacagacgcatgcgtgtccacggactgccccaccgcgtgcatcctccgtgcgccgattggtcagcctggctgtcaatcagagcatcgagcaggcggagcttcgaggacggaagagccaaactttcctttggctgaggaaggtaaagtacggtctccggctctgctttgggggaaactgaggcaggaggcgcggttatttactcgcggtagaggacgtgtcttagaatagaaagaggcgagtttgcagatagattgtttcagttctcaggacccttaatctgaaacccatccttgtaaagcagagggagaaggtgaagccccacagccttcgggccagggcacctgcatggaaatggtctgcgacattaaccatcccatgactgtggctcaggtggtagagagatttttctaggatgcacgagttcctgggttcgatccccagcaccacacgaagaccaagatcacagatacagactgagttggagaccagcctgggatatgtaaggcccttctatcacaagaaaaacaaactaggccgggtgtggtggtgctcttcttcaacagaggccaacctggtctagacagtgagtttcaggacagccggagctacacagagaaaccctgtcccccccccccaaaaaaaaaagaaaagaaaaacaagcttggtgcggtccacatagtaagttccacactggccagtatgttgttaattccaaagatggaatgagtgtaaggaaaaaggccagccaaagaaacacactttggctctgcaaccagaaccaaagaaatgcacccttgtcagaagccagccttggtgacgcccccccccccaatgcctagctagccgccagctagcctagcatcccacccctcaggttccaggacctggcctgagaagcaactaacaccttcccccatttccttagttagtgcttgagataaacataggagattccagatacctggcctgagaagcaactaacacctattctttctccacacaggagattccggatacctgcctgagagcaattaacattcattcctcccccacctcctttttctctgcttcctcctttctctataaaaaccccttgtaataatcaataaatgggccttgacaagaaaacctgcttggtcccgctctttctttcccgcccatcattttcaggcgtgatccacctcggactcaggaattaccggagccccgagggccggggtacaccctgaccctgaaaccagtgccagagaaacacactttggccctggaatcagagccagtgaaagaaatacaccctggctctaaaatcacagtcaaaaggctaaaaaggaccagtgaaaggaacacactttggccctgaaaccagagccaaatctaaccctaaacctgtgcagaaaaccaaccaatccctgagcaggagatctagccattctgagctcagggattgaaatctgaccaatccccaccctggaaatctccctgggaaaactctgcccccccctaagaatccctatataaaccctgtgcctgttcagcttcaggctgcctggctgccctctgccactggattcttgaatgaggtttgggtaagaactttcgccggagcagagaccaccctacacactggctctccaggggactggagttgaactgttacaatttaaccagggaaaccctctcctccccaagcagagctgatacactgaggaaggcctttccctgaagcagggctgtaagccgctatgcttactgtgacctgtggcattccttggctcctaaacgccagaatacctttccatcccagctgtaacactcagtattctgtgactccggagtgccagaatactttgcatctgagccataacacagtattctttggctcccgagtaccagaatccattccttcccatccaatccgttgaaatgcttacaatgagaaaaaccacgaacccaaatcattgagaccaaattaattaaagtttttttttcattcatgtacgagactgcctcccccaaggcctgatttgagaggtcagcattggatgtgaggaagacaagggggttttgttgttttggttttttgagacagggtttctctgtgtagctttggagcctgtcctggaacttgccctgtagattaggttggcctagaactctcagagatctgcctgcctctgccacttgagtgctaggattaaaggcatgtgccaccaccgcctggcctcacctggatcacacagacacatatatacataattaataaaatacagacctggcctttaatcccagcactaatctacatagtgagttgcaggacaaccagagttacatgaccctgtctcaaaaaaaaaaaaaaaaaaaaaaaaaaacaacctaaataaataaaatgcagaaagtaacaatatgtacataataaaaataatgaaataataacataattgaggaacagtggggcatggtggcatgtgcctttaatcccatcacttactgatctacatcccaagcctgaaggaccttgctgaaagggctccagcactagactgttgcttctggcagagaaagtggctctcaacctgctggtcaacgacccctttgggggtagaagggccctttcacaggggtagcctaagaccatcagaaaatacagatattcacattacaattcataacagtagcaaaattacagttataaggtagcaacaaaaataattctatggttaggagtgggtctccataacatgaggaactgtattaaaggttgcagcattaggaaggttgagaaccaccaatttaggggcagatgctgcctccttcacagcactcagccacagttgtgttgtgtccttgttgaggttctatccggtcctccaaagttcagctgtgggaaactaaatctttaacttcataataatggtaattggaggctggagtaattagtaccagataaaatcatgaagagtgtgcttccatgatggccattaacagctttctaaggagagcagagaaaaaaaccttttcctcgttgtggaatgacttcctccgtgttggtgtgcaccagaaaggccccctgcttgtgctaagtggagctatgccatgctcttggactctgcagcctctagaaccatgatccagatcaactgtgtgtctgtgtgatttgttttatttttattatgtgtgcatgcctgtgtgtctgcgtaagtgtataccatatgtgttgcaggtgcacggagaggccataaaaggggcatcagatcctccgaagctagagttttaggcagtcgagagcctccacgtgggtgctgggaactgaactcagagcctctgcaagctcagccagtgctcagccagtgctcctcagtttgtgtgcttgttttcagtactgcagatggatccaggaccccacacgtgccaggcaagcattctctcactgagctgcaccctcagactggtctcaggtggtttgttatagcaacagaaaacagacatgtggaaggagtgatggcgtatcttgactgagaggaggaataacttggctactcgcacagcacatgtctgggcaggttcacaaggaggcttccagggacaattggcacctaagaaccctgacctaatgaatggatgaaccattcagaatttggatgggttcttgggggggggtgagggaactgtggtgggtggggactgcttggaggaagtagggtgtttgttgtgggtggctctcggaggctatgtcttacattggctcctttctgttttggttctttcaatttcctgttgtgtgctattctgctacaaccgcctcacccagtagattgcagcctctgttaatcaagacagggtcttactgtgtagccctggctggcctggaactcatagagatctgtttcttctgcctcttgagtgctggaattaaagacgtgtgccaccaccaccacctgacctagaggacaactttattttttttaattttttatttattatgtacacattgttcctgcctgcgcaccagaagagggcaccagatctcattatacatggctgtgagccaccatgtgggtgctggtacttgaactcaggacctctggaagagcagccggtgctcttaacctctgagccatctctccagccccctggatgattaattgtagaagtttcagttctttctttttgtttttgttttttgagacagagtcacactacgtatcttcaactgatctggaacatgctacgtagacaaggctggcttcaaatgtgtgtcaatgttcctgcctctgcttccagagtgctgggattataggcatgtccctaaatctgttatttttttaaacacttatttttgagggggagtcggtggtggtgctgttgtacgtacatatgtagagatcaaagggcatcttgtgggagtctgttctctccttccaccaatgggttctagggactgaattcaggttctggagcttgacagcaagcaccttaatctgctacaacatcttgttggtactaaatccatggagaattgaaaggattgtttattgttgggcccgggaatatagctctgtggtagtagaatgcttgcctaatatgtgtgagattcttaaaaaatcattgattgttttcttaaaccaactttttaatttgttaaaaccaacttttaaaggatatatctaggcatggtggcacacagcattaaccctagcacttgagagatagaggcaggcagatctcctatgaatttgaggccatcctattctacgagttccagtacagccaggactacaggactgtgtgatatgcacaacacaggaaggactgatgacaaatttggtaaaaaaaagaaagaaaaacagcaagtggtttcacattgtccggcttcacctggagtgggtgactgtgggtactcctgaagcctcccaggagtgggtgactattctgtcagactgtgggtattcccgaagcctcctaggaggcctcctgtgcatcacagaagtggcttgagcaagaacttgggacagactgggcaagtgcacagctataatcccagcactgcagaggtgagggctgatgggtcaggagctcacagttatccttgactacaggaagtctgaggccagtctgagctatgtgagactctaaaacatacatgagtcagtgagttggctcagccagtaagggtgcttgataccaagtctgaccacctgagtctgatccccagaatccacatggtaggagcagagaaccaactcccaggagctgtcctttgacctctggacttatgctattgcatatgcataccaatacataactccctaaaatacaaaatacatatgtttgggctttttgtttgttttgttttgttttttcgagacaggtttctctgtgtagctttggagcctatcctggcactcgctctggacaccaggctggcctccaactcacagagatctgcctgcctctgcctcccgagtgctgggattaaaggcgtgtgccaccaacgcccgactgatgtttgggcttttttgatggtttttatgtgtgtgtgtgtgtgtgtgtgtgtgtgtgtgtgtaattagaaggagaaagagagagagggagagagagacagagagacagagacagagacagagacagagacagagacagagagaggagagagagccaaagtctggtttgtggcccattcaactaagcagacttctcctccaccaatggtgtccaccaatggcccaggaaaggccatttccccacttgaaccagcgcctggccctctggagggttagttcctttactctttggaggtctccctccctgccccatagcaggctgccctctcttgctcccttcccacaggcagctaccttcatcccagtgggctatgagctcctggaagaaaaggaggagccaaattttgcctaggctgagggctcagtgccaggtggcagccacagtcaactgctgaacttgtgaaactggaccccaggagaagaagcctgggacaaacatcagcttgcatcagctccctctggctcagttacagctttgcagcaggtgtggacaggctggtgcttcagcaatgggaaacaggactgatgcaaaccaggcctccaggaggccaagccctggagccagcctgcagtgaaccagcctccagccacatctacaactgtgtgagggcctagaatagaaagtgtacttgacctgtctgtccttgagtgctcttctcttctcacacttggctccaggcatggggcagcttccagcgatggctttccagacaagcttgagtaacttggccctttgtcttagtgttttctaaatccaaataggctttacagggaggattgtgaaattatgttagctttggatctgagagccagactgaaacctggcttcctacaggcctagtctttcctgcttaggtcagttacctgtcactaataaatgggggctccttttcctaccagccaccaagatggctgaagtggaggagaagaaatgaaccttccacaaattcacatactacaatgtggacctggatcagctgttgacaggttctaggaactgctgatgcagttgtacagcaccccagaggtggtgtctgaaccacggcctgtggcagaagcagtactcgctactcaaacacctgagaaagctcaagaaggaggcaccaaccatggagtaacccgaggtgctgaagacccacctgagggacacgatcatcctgcctgagatggtgtgtgtgtacaatggcaaaaccttcagccaggtggaaatcaaaccagagctgatcaactgctacctaggcgagctcttcatcacctccaagcccatgaagcatggccagcctggtattggtgccacccactcctccagctgcatccccctcaagtagctgtggccaacaaagactcatgtttaaaaagaaaattggaagccaggcattggtggcacacgcctttaatcccagcactcgggagacagaggcaggtggatctctgtgagttggaggccagcctggtctccagagcgagtgccaggataggctccaaagctacacagagaaaccctgtctcgaaaaaccaaaaataaataaataaataaataaataaaataaagaaaagaaaagaaaagaaaaatgggggctcactctgagaagacctgactcctcctcctcctcagaactctctggtttgtttttgcagtgctggggtcactgtgccaggctagagactcactgcaatctgagggtggaagtgctgagcaggaacagggaactgtgtataagctggcataggcattgataaattcccctgtagatggaccaggacctttcaacccgaacacatggaaagttattgtaaaatacaacagttgggaatcaaaagagtggtttgatccaccttacaggcaaatttcattcggaaactgacaacacatatggatcatgtggtctgcctgaatatcaaatatggcaggcctcaaaaatcaactgcagattttaatataacatttaattattcatctattaattaatttattctgtctcataatatcaaaccttatctattaaaagggagcagggctggggatgtggctcagttggtagagaatttgcctagcatgctggaaaccctgggttccgggttcaatccccagagccacataaattggatgtggtgtttcacatctgtaatgctagcacttaggaagtggaaacaaatggatcataagttcaaggtcatcctccactacataataaatttggagccagcctaggcttctgtatctagaagaaaaaacagggcaccactgatgcaactcattgcataaaagcaattgctgtgtgagcctgacaatccaagctcaatccttagaaccaagagtggaatgaaagttgtcttctgggccatgcacgcctttgatcccagcactcgggaggcagaggcaagtggatctctgtgagttcgaggccagcctggtctacagagtgagttccaggataggctccaaagctacacagagaaaccctgtctcaaaaaaaaaaaaaaaaaaaaccaacaaaaaaaagaaggttgtcctctgacctccacacctgcaccatagtactggcataccaacacacacacacacacacacacacacacacacaccagtaatgacaaatgctatctcctgtaattctcggcaaatagtttcaaacaaagaacacaggcaaagatgaggtattggcataaataatttacttcaggctctaataccatacattagtggggaatgagaacaaaaggaggaattgtctgacttcccctggggatcacaaactctatccattcggcccagcaagggcgccaatgaaaggtgaagaagccacgattccatccaagtccagcttggtgaaccggtgagtttactaggttacttacaggtggggcctgggtgactcaaaatcacaggtgactccctccaaatctgcatcagtgacatcctggctttagttaaccttttacctcttatatactctagcaccgccccaagatcatgagcagctggggcggggcaggagggaggaatggctgggatttcaggtgctaagaccccctgacactctcctccctttctaatacaggtgttaactatttccataccctagccataggcctcaccgtcactgtggcatttggttcattttgttgtcttgattcaggtcaaagtattctggaggccaccatagcaatgtctgttgcttgatgagcatggtcaaggcaggaggggactgcagagagggagtggcacatagggatggcatgtgaagaccgagtggcagacttggtgccaaagcctgccagggacaagtgtttgtcccctaaactacctgtgacatgaaggaaggaactgagccaggctaggaagttctcccgtgaccagcccaccccagagctccagcccttcctgcagtttccttggtgctgtgctgtgagaggggtgcaggttggtgagaagctctccagctgccaggcttgtgggtgcttctagcagagtgcaaggtctccagtcacatccttggctggggacggcatctgaggacttgggccttcattgcagcatcttcagacaggcggggaggagggaggaggtcttggccttggacggctgttcttcctcagtggcatccaggaactgctgcatataactggaggagccaagcagcatgtggcctgtggcctgcatgctgaagagggcccagcggagcatttccctgggagacagcaaggcactcaggttaaacactccccatgctatctccccaaatgtcttctccatttttctatgctggcctaaagcagttggcttcaagttagacttctttctttgtttctacttcttttatttttgagatagaatcttaacactttatctcaggttagctagaaattcatttatgtagctcagtctggctttgaactcacagaaatctcctgcctcaatctcctgatgctgttgcagttatgggccaccacacctaactttttttggtttggtttggttttgatttggctctttttgaaacagaatctggagatcctcctgtcttgtcctcccaagtgctaggtatacagacatgtcccaccatactgggctcaagctagcttttctccttaggaagatttaagtgtcactaaagtaaaatgacaaaaatgcttccaagtacagacaggaactgggaaccaggctcatagctagccatcccttacacatagttctgcctctactcttgtgacaacttttctggaaccccttgctttttcccatgcttaacttgagttctcaaaacaaagctttttattttcttgtaatgatattttgtttctttcttttctcttctatggtgctggggatggaacccagggctttatttgtgcataagcagctgacctgccatggagctatgtccccagccccatatattttttttttcaagacagggtttctctgtgtagctttggagcctatcctggcactcactctggagactaggctggcctcaaactcacagagatccgcctgcctctgcctcccgagtgctgggattaaaggcgtgtgccaccaacgctcggcatccagccccatattttttaaactactctggaccagccagattaatttacataacacatgtctaccctttgtaacactgcttcttagactttatagtcttcccaggccagttctcagaatgctggccacaaggctcccatgcctgatatcatattctaagcatagaacttggaccagacagggctgaacactgatccagagtggtttgtttatatctaaaatatttaaaatatattttaaaatattataaagatgggtgatgtaactaatttagtgcttgcctttcatgaacaaagccctgggtttgatccccagcaccgcaataacccagagtggtaatataggactgtaaacctaggatccagcactgtggaggtagaagcaggtggatcccaagttcaaagtcatccttggctacatagcaagtgtgagaccagcctgagatacatgagaccctgccaaaaaaaaaaaaaaaaagctttaactgttctggttctgagctccagcagccagaagctttgtgacttgtaaaatgggtaatgagtttgaaactaatgtgaagcacttagcatttggtctgatggacagaaaatgggacatgcagccaagagcagctggctgaatgcaacttctcccacagctattgacaagggctcggcaggggtcactaggagttgctggtcttaagaacaataaggaagagaaaacacaagaaagagaaatactgccaggccgcggtggcacatgcctttaatcccagcactcaggaagtagagacaggcaaatctctgtgagttcgaggccagcctagtcttcagagcaagttccaggacagctacggttacatagtgataccctgtctcaaaaaagcatatatatatatatatatatatatatatatatatatatattgaatttagtggcctgaggcaatggctcagtgggtaaagtgcttgccatatggccatgaggccctgaaagcccatgtaaaactggggatggctgcctgtatctgtgaccccagtactcctctcatggtgagacagagagacaagagactcctctgaagctttcaggccagcaagcctggcccagacaacagacaaacagcaaagaccctgcttgaaacacagtggaagatgagaccagcaccttaggctgtcctctgacttcgaaatgcacgctgtggtacatgggtgcccacattcacacacatatagagataaagactggctgggcagtggtgacacacgcctttaatgccagcacccttgaggcagaggcagttagatctctgagtccaaggccagcctggtctacagagtgagtttcaagacagccagggcaacacagagaaataagaaaagagaaaaaaaaaagatataaagactgaaactgaaaatataatatattagggctagaagtatagcttaatggcatggtgcttgtctagcatgtatgaaggtcctggtttaatccccagctttgaagcatgtgtgtgtgtttgtttctttttgtggtactggggaactggacacaaggccttaggcaagtgctctgccattgaactacagcctcagctttcttttcttttttaaaaatgtttatttattttatgtatatgagtactctatctgcatgtatgactttatgctagaagagggcgtgagatcccactatagatggttgtgagctaccatgtgggtgctgggaattgaactcaggacctcaggaacagcagccagtgctcttaaccgttgagccatctctccagccccctcagctttctttttacccccccacacacaatatctctctaagttgcccagtttagccttgaacttactctgtagcctaggcaagcttctaatttgccatcctcctgtctcaatttcctaggcacctgggagtacaaggccatgtatagctttatatatatgtttcgtgcagtgccttcaaagttatttctcaaattagagaactgagttttgactctagccagccagtcttaaaaaggaatgaaaataaggcctattctacaaatgaatgaaccttgaaaacagaattcttgtggggaaagaatcatgatcccatttctatgaggtctctaggatagatcaatggagcaacagaaagtagagtaaaggtgagcagggtctcgggagagggctgagaaccattatttactgagtacagcttctgctgcctggtgcaaaggttctggaaaccaaggcaatgcttgcacaacatgattagttacttcatgctagacacttcaagcggccacagttgaaaaatgttgaatgtgcagattttagcacgggtgttttttcttgaaggtctcattgtgtagctctagctagaatttggaacttgaaatgtagaccaggctagcctaaaattctcaggagatctacctgcctctgcctcctgaatgctggctgggattaagggagtgagctaccacacctgaccccttagtacatttttttttttaaatcacagtaacaccactaagtcactcagctaaccacagtttgacatcagtttttattttctctaagtttttatttttgttgttgttttattttgtttttcaagacagggtttctctgtgtaacagccctagctaccctggaacttggtctgtaaaccagactggccttgaactcacagagactcacctgcctcagcctcccgagtgctgggaccaaagatatgactcctggcctttttattttctttccatgtgtgggaggtaggggttatgcacctgagtgcagtgcccacagtggtcagaagagggcaacatatctcctagagttgcagttacatgtggttgtgagctggctgaggttggtgccgggaactggacctaggtcctcgggctgagtggttccctttttctgtttttgttttcaatacagggtctgatctggcccaggctgtccttgaactcctgattctacacctccaaagtactggaattatagttacattttcacttacaaaaatattagctgtggaaatagctcaggatatgagtatgtgcttagcatgcgtgctgccctgggtcctatccccagcacacaaaagaaagcacatgactgtcattctttgttagcccccagtgccaacccaggctttgagagagttcagctttgggtagacacaaaggacctctggttagctagtctcccaccctgctgtcccaatctactcacttcacgacgcgcttggcccggtagcagtgcaggtcataggaaagtgtgtatgtgtcatagagggtcgccttcacgttcaggcagccgatgaagtcctgggggttcagcttgtataggtcaaaggttacccgggccacatcaatcttctttgttggcttctgggaaagggatagctgtggtctcgtcttgcgctgcaagacaagtccatgttagctcgcctctgggactccttccacgccctcgacactcacccctgctggaggagactgtcacctgttctgatgggggcttccacttctgccccttctgcaggaccatgaacacagtatctcttgccagggcttggaagtattcttctgtctcaacaatcgtgccgtcttcctccagcacgagggagaagggcttgtctttaagtttcaagatatcctgggcctggaaaagaaggttggtgacatttctgccatccctatggagccacagagttgaggagcaaaggcccgcagtaaagcacttcagctgccagagagagagcaagtgagctgccatccatccgtgcgatggactgactgtcactgtgggggcccactcaagggctgtgtctcagtacttcctactcccaagaaggagtgtagtctaatagaatgggagtcatatgtgaggtcttttttattgagaaataaattacataccatgctgggcaatattaggagtcatatcgaaggccttgggcaagctaggcaaactgcagcattgaactatatccccagccttctttgtactttttgtttggagacaggatcttactaagttgtttaggttggcctggaactcactgtgtagtataggtaggcctcagccttctatttagatcttcttgactcagcctcctaagtaacttggattacaggcctacactaccaggcccagctaaactttattttgttggtggtggttttttgtttcttttgctatgtagccgtggctggccttgaactcacagagatccacctgcctctgtctcccaagtgctggggttaaaggcacgttgttatggaataatcttttggtacactgtgaagttgtgtctttgtcaaggcgctttctgactggtttaataaaagaactgactggccagtagctaggcaggaggtataggcaggaaagcaagacacagaggactctgtgaagaagggcagagtcttgggagtcatgagcaaatgcagagggaagcaagatgaaagaaggtaccactatgatgcagagggtagatagtaaaaggattaatttaagtcatatgagctagctaaacacaactctaagctatcagccaagcatttataactaataatgagtctttgtgtagttatttgagaactggctatcgggatagaaaagtctgtctatagtgtgcatcaccatacctggccactattttaaatttttatttttaattatgcgtatatgtatgtatgtgggtcccctggagcaagtgattgtgagctgcccagtgtgagagctgggaactgaacctctgtcctctgaaagtgcttttaacgactgagccaccactctagccccaacaactcatttgtaatcttgttcctgccaaagctacatggcatgaactgaagaaacaggtcattcaaaactgagggacaggaaataaaataactggcttgccatctttaaaagccagctagaagaatcagcaggtaagggtacttgcagtgcaaactttagcatttgagttcaatacctggagcccacagtagaaggagagaaatgactcccaaatgttgccctgtgacctccatgtgtaccctgtggcatgggcatgccagtgctcacacacacgcttcatagacttacagtaataattttaaatataaaataaaactgtctggatttgcaggacaatcagcagtgctccaacagagtctcaagataagcttgggggggttcttgttatgtagccctagttgggcaggaacccctatatagagaccaggctgacctcaaacatgtggtcctcttgcttcttcatgcatcaccacactcagccttagttttaatttcttttgaggcatacccagcgtatcctggaactcaattatgtagcccaggctagcctcaaacctgagatcttccatatctggtctcccagatacgagccaccatgcctggctcatttctccacgtgtaaatatggggaaggggaatgaatggccctttcagcaagaaaataaccacccacccacggttggtgtgaaagacggtagtgcactgccctccttctgtcacttgaccatgtcacttatcacacacacagctctccaggcggaaatgccaaccctccagtgccaacacctttctccgcttaagaccatgccactcatgcgcacagacttcagggctctccagaaaccgaagcagccggcaggctcaactctcagccaaggcctggagagagcctatgcagggtaccggcagatcccttaccttgcccaggaggtcctccaggctgtgagccatgatgcctttgcgaaccttccgatctgccgtgctaactcgacagggcctcgccctggggacttcccggctgggcttagacaccagctgttgggtcaccactgcagtgctcactgctacatgcctggggacaacagtttgtacagcagatgacctgcctgcggctaccaggtctgcccatgcctgcctgcttgctgagttcaggtgtgacctttccttccaaccaatcatggctgcttcaggcggctttcacaaagtccatcagaagacacagctccgatttggaggggcagggaaatcagcgggaatcttgcttaaatcagactcacttcagtgcctctgagtggagcccaaggactgctaacaagtccccaagctgtgtcagtgtggctggttcttggaccagccacattctcctgtggatggcttctgcttttgtggccttgttcaccttgcctaagaggcagggtgaaatggagctggtgtgtgtatatatgtggggtgtgtgtgtgtgtgtgtgtgtgtgtgtgtgtgtgtgtgtgtgtgtgtgtgtgtgtaaatcggcctgtagaatatgtggggactctgaggtacacagggtagaaagaaaggagcagagagaatttgggtcttggctggaattccaagtttctctctgttacggatttgctaggtgacagcatacccagcctgtgactcagtttcctcatctgttgacttggcatagttgccaccacttcccagagaggagtgaggctcctgggagatcaggaacaaaggcacctggtgcccagcctgaccctttctactccagatgtgatgggaactgtggcagtgacagagagtaagatggggactgtctagactgcaagatcttgggtgctaccctaggcctcctgaatcagatctgtgggggtttattgtaagattttgtctgttttgttttaatgggggtcttgcttatttgtttgttttttgggatagcatctccatatatagccctggtttactggaactaatttcctcatctctgtctctgtctctgtgtctgtgtctgtctctctctctctctctggacctggacctatgcagaccagactcaaactcaccaagatcctcatgccactgctccccaagagccaggattcaaaacatatgccaccatgcctggccatatcagctagaccccacatgactttttttgtcttttaaaatgggatgcctatgtagccctgactgacctgggacttgctgtgcccactacaggctggcctcaaactcagatctgcctgcgtctgcctcctgagtgctgggattgaaagcgtgtaccatcacatccagcccctacatggtttttatacctattaaagttttcaaagttctaatagatggctccccagctttccccttccttcccatacccagctctccttacctggacagtgacttagggtacaggaggctgagagacttcatggcatagtccatccttgtcagctggattgtgttggacctggatcggaggcagtaacctatctggttagagtgggaagggagggaggggtccctctctttctttctttgactcagtggatcccagcccccacacggcaaccctctgccccagtctctcctcctacccccaatttccctgaatccacaccaactacactgagtcagcgtctaccatccccacccattccacagccccagaacaactcactccatgtttctctgccccaagctctggttacactgagctgaggaacccaagtcaaggctgaagaggccctcactgatttgtccctgtccctgggggcagagtccacattatgctggcaggaggtgagtcataccacctcatgctgtgagagacttgaaagtcacctattgtccagaaactccacaggcagtcagaccttggagagcctgctctaaccatgggcccttgagcaataaagtcctcctgaggcttagaactggctaagagaactttctgtgatcttaaagactgtctaggcttgtgttcaccaataccttagctgttaccactgagcccttggcaccataactaatgtgacaaaggagctgacaccgaacttctattgaggcttaactaatttagttaggtaactactgtggtttgtgacgccaggtaccccactggacattacagttgtagtggagtaatcctgggctcatgtgccccctgcctttcctcctatattgctgttctctggttcttgcctgctgagctgcaagtctctgcacaggcagtctcttctacctgcaatgcttgtctcaaactcagagctcaacataagtcacctagaagccccagctcctggctgccaactcaggaaagccttttctggtgccctcttgcctggtaccagattgatccctgtgccctgcccttcc

SEQ ID NO: 5 - DNA, 29980 bp, Cricetulus griseus
  misc_feature 1..29980: note = 3' of SEQ ID NO:1
  source 1..29980: mol_type = genomic DNA
tgttcatttctgctgcagctctcgggtccccactgtgcctgttttcagcccgtagcacagggacccagtgtgaacaccaggagtgggcaggcatggagaactgcctcctcagtgaaggaaaaccattcttcccttctgataactcgggttctccccggtcaaccttacagagttagagaccctcgtccccacttaatgctacccaagcactcttgtttcaccctgtttctaatacccaccactcacacagctccacaaaacccaccacacctgctctgtggtagccaaataccccagctcttctcctccccctcacctcctagcctcctggcaatactgcgtggccaggcacagcccaggcctcctccattttatttctatctcctgtgaagccactcccttggccccagccccagctggggaaaagagcacagggagtgagtctgaaactctccctagggatttcaagccagactgacttccagaaagccccgaaagggggaagaaggcatgtttcgaaagctctagaaggaagaggtactgcccaggacttggtaccaggcaagagggcacagaaagcaacagacggggcttgtggtctgcgttccacccctcacaccagctgtttgcgctcacccgagttgccttctttcagccctggattttctcctgtgcatgaggaataacgctcctgttgaagccaggaatggtggctcagagctgtaatctcagcacttggtagactgaggcataaaaagggccatgagtttgaagctagcctgggctatagagttaggtcctgtctcaaaaacaaaaatatagtcagacggtggtggcacacacttttgatcccagcactcggaaggcagaggcaggcggatctctgtgagttcaaggccagcctggtctccagagcgagtgccaggataggctccaaagctacacagagaaaccctgtctcgaaaaaacaaaataaataaataaataaaaaagaaatatctttttttttttgagtcaggcaagatacttgagttttattatgtctgtctaaaagcaggtgttaggcctatttgtagtgaagcgtggtgtgctggcgctgcaacacccggtgaggaagcaggaacctgatctcagagcccaagaactgcttgacagcaggccttcggcacttgccctctgaaatctcttccaccttcatcaggttaatggagtgtgcacgggcacagtgcgggcacccgtgtcttggtagcactgtttgacggctccagcagcggtcaggtcccggtattcttggcacatgttgtgtgtgtcatgttgtgagttgtagtgcagccataccgcaaagttcttcacccgcagtggggcctcaggtcgcactgcacagtgtacgatctcctcggatgacttcttcatcttgttcatctgtgcccaaaatcgggacttggccaccacatggttcgtcgcaaagatgtgcatgaggtataggagtggtgtgtggcacttccgggtgggcaagcagcgccccaccaccttgtactcccgaagcgtgcctgaggccttcgtggctgatctgtccttgttggccgccagccacaaaaataaaaggaaatatcttgcctataatgttacaatcccatccagcacttgggaagccaaggcaggaaggcctctgtggatgagaccagcctgggctacagagtgacaacctgccaagagaatggggtgggtaatcccaaatcttgggtatagtgaagtacaggactcaagagacagaggcaggaggatcatctccagttcaagacaagcccgagctataaggcctcaaaaacaaaaaaagaatattagatctatgatttattgctataaacactacaacttaaaaaaaattaaaataaggacagcaagatgactcagtgaatgagggtttgaatttgatccccagtacccacatagtacaaagagagaactgactcccataggttgtctttccgtgaaaactctccctcccttcctccctctctctttctctctctctaaatgaaaatgtttacatcaaagcctctacaaagagcattcatgttacacctagcgttggagagatgtctcagtggtcaagagcgctgacttctcttccagaggatccaggttctattacaagtacccatgtggcaattcacaatcatttataactcagttccaggggatctgactccctcttcaggcctctgtgggccttcatgtgtgtggtgcaaagacttgcacgaaggcaaaacactgtacacataaaataaaaataagagacagaaaagggaatttattcagtgtggccataccagaaaagatgagaggaccagtctcttcaaccctgtttttggagtgcagatggtagctctaggaatttacaggagaagaacaaaaaatgggatgagctggtggtgcaggcctctcagtgcttgagaggcagaggcagaggcaggggaatctctgtgggtacaaggacagcctgactgacctggtggattccgggctaaccgaaactacaaaataagacactacctctaaagttaaaacaaaaaagggaggcaataagtatgcatagctaagcattctattctattctgcccaaaccactctaattgacggctacctccagtctgaagtaagggtcctgcagtagctaggaagaggactacctcccaggctgactgttcctggctctggcaacaaactgaaaaggaaaactggttcagaaggagatcagagcagaggggccaaattagaacgaggattgtgccttccttcaggtttagaacaaccactggagaattttaggtactgctgtggtagtttggaacaagacgaggcaaaaacttattactggcaatctgtaatgtccccataaatcctttttatatctatttttaaaaagatttctatattatatgtacagcattctgtctgcatatatgcctgcaggccagaagagagctccagattttattacagatcgttgtgagccaccatatggttgctgggaattaaactcaggacctctggaagagcagccagtgctcttaacctctgagccatctctccagccctctattttattttttaaaagaattattttatgtgtatgaatatttgcatgtatgtatgtgcaccacattcatgcagtgcctatggaagccagaagaggacgacagattccctgaaaccagagttacagggggctgtgagttgctttatggtgctgagaactgagcttccttcctgtacaagaaaagcacatgctgttaaccactgagctgtccctccagccctccccacccctttgtgtgtgtgtgtacttgtatgtgtatgtatgtggacatgtgagcatgtgtggaagtcagaggatggtttgcaggagttggttctctccttctaccctgtgggtcctagggataaaactcaggctggtagcatgtccctctacctgcaaagccatctcctgggctttgtcccaataaatttcttgctctcccttggtatcttcattcttaaagggaaggacagagctacaggtacaatgggtgaaaattggcctttagttaccagtcttgaagatgagtacttaaatttttttcagaatcctttttttttttttaaatctttttatttatgtatacagtattttgtctgcatgtatgcctccaggccagaagagggcaccagatctcataatggatggttgtgagccaccatgtggttgttgggaattgaactcaggacctctggaagagcagacagtgctcttaacctctgagccatctctccagctcaagaatcctttttttttttttttttttttaaggcagggtttctttgtgtagccttagttgttctggaattaggtttgtagaccaggctggcctgaattcaaagacccacctgactctgcctcccagggtgctgggactaaaggtgtgtgccacgacacctggctaagatgagtgctttttaaatgcctatcacatctacaggcacaactgattgggaagctgattcaaaccttgagatagcaaagggatagaggtgttgatgcaacataaagacagagatgcccagattttctccttcctttagtcactgggcgggactctgcaggtccagtgaggactcactggctctcttgttaaagccttatttagagttaagccaatccttggaagaggcatcattcattcattgaacacttgcagtgtgttaccctgttaattcagcagaaagcaagacagctttaggccacacctggagcagctggaccagagaccaagtggaagcaggaatgctgatgtacacttctcatcctgatcccctggaggctgtcagcctgggccacacagtgcgagatcctgcctcaaaaacgtttttgctacccattggtgattgtacatgtactttgttctagcactcaggaggtagggccagcctggtctacaaaggacagccaatgctacacagagaaaccctgtctagaaaaacaaaacaacaacagaaacttttttgtctgatgtttcatcttgtttcttttctaataatgtactgggcagagaaaaacaaaataatgcagttgggcctggaggcttagacctgggattccagcacttaagagatagaggcaagctcattgctttgagtttaaggtccctctgccctgcatagtgagttgcaggtcagtgtggactatagagtaagaccttgactttcaaaaagcacaaggggccaggcgttggtggctcacagctttaatcccagcactcgggaggcagaggcaggaggatctctgtgagtttgaggccagcctggtctacagagggagttccaggacagcctccaaaacaatacagagaaaccctgtctcaaaaacaaaacaaaacaaaacaaaacaaaaatgtcaaaaagcacaagggccatcaaaatggctcagtgggcaattacaaacttgatgacctgagtccattccttgagacctacataatggaaagaactgactcccctaagttgtcttctgaccttcacacataccgtgtgtgtgtgtgtgtgtgtgtgtgtgtgtgtgtgtgtgtgtgtgtgtgtacatgcacgcgctctcgtgagcaagtacgctcctgatactaaattaataaaaaggattaaaaaagtaaaaccacaaacatgaaccaaaccaatgtggtgtaataacctatgtctggagaactaacaggtttctggacccagagaggtagagaagataatctaatgtcttcaggggaattagggggataaagcatcagagcataaagaaagtttccctcacttgggaagtagtggaggcaggagcatcaggaattcaaggtcatctcatctatatagtaagtaagttcaagactagcctgggttacatgagaccctgtctcaaaaaaaaaaaaaaaaaaaaaaaaaaaaaaaaaaaaaaggaagcatggtgcaacagatttgagattgaaacacaaaaatagctcaactctggggcagacagcagagtcaggggaaagtctaaggaagttctgggaaagggtcaggaggcccctccctgcttcattcacgccccaaacatctattaaacattccatgtgtacttggtgtttctggcctgaggatgactcacagaggctgacacactaagttaatcacaggaagcacagaagaaaaaaaaaaaaaaagaaaaaacaggcctaatgcaatggaatgcctgactgagggggttcttcacagtgctttggagacgaccctttgtgtctcattgaaattcatctctcaagaaacactcctcaaatgtttacacattgtcaggcactttgagctgcagggacacagaggactcagttctggtccctgcctttggaattcacaattgttgctcttccaaagaactggggttcggctctcagcacccacttaaggaggcccatgaccatttgtaacttcagctcaaccgcttccacaagtacccacacacatgtctgtcccccaaacacacctatattcttcatgctaaccttgtgacactatgagcaaaatcaaagacattaagaagccaggttcagtgccctgtgccttagtcctaacatttgagaggctaaatcaaacaggaattgaggccacttggacagcatcaagagaaattatctttgaacatatgaaatgatattatcacagaaatagaattgcaatttaaaccaggtatgtgatcccctctccccccaccaaaagaaaaaaaaatagagggctggagaaatggctcagtggtaaagagcacctgctgttcttgcaaaggatctagattgttttgttttgttgagacagagccacactatgtaactctggctgtcctggaattcactatgtagatcaggctagctggtatgtatgtctgtccacaaatgcatgcctggtgttagaggcttccagaagtgtgtgtcagaccctctggaactggagttataggtggttgtgagctgccatgtgggtgatgtaattgtacttgggccctttgcaagagcaaccagtgcccttaacccctgatacttgcttttaaggaatttgcatttttgttttgaaattacatgttggaaaagttttccacatcagtaagaaatgccatccttactatttcttcctgcagcttctcagaaatattttttaatctttttttttaagattttatttatttatttattatgtacacaacattctgcttcatgtatatctgcacaccagaagagggcaccagatctcattcaaggtggttgtgagccactatgtggttgctgggaattgaactcaggacctctagaagagtagtcagtgctcttaacctctgagctatctctccagcccctcagaaatattttaattcaaaaatctttcttagccagctctcaagccactggattacttcttgcttctgctactgacatagacttcatcttgattcactccatacagcaactagatgtgtgtatataaatgacagatagatcatagatggatggatggatggatagatagatagatagatgatagatagatagataatctcacttgctacctaggctgacctcaaactcatgcctcagtttcctaagtgatgtgattacgggcatacactattatgcctagcataaccatttgtttgccttaaaattttttaagattaattttttattatgtatacagtattgtgcctgcaggccagaagagggaatcagatctcattacagatggttgtcagccaccatgtggttgctggaacttgaactcaggacctctggaagaacagccagtgcttttagcctctgagccatctctccagcccccagcctttaaaatttttaattaattttttttttgtttgtgtatgggtgtttggcctgcatgtaggcctatgcactatatgtgtgtagtactcacctagaccagaagagggtgccagagactctggaattggagtttcagtgcatgatgagctgccaagtgggccttaatccccaaaccccagagggttgaagagggatcagtggctaagggtgcttgttcttgcagaggacccagttttgattcccagagcccacatggtagctcagaacaagaactccggcttcagaggattctgcaccctctctgggcctcatctggtaccaggcatacatctggtatgcagatatatacacgccctgagtggtgacaaacactctgtgagttcaaggctggtctacatagagaattccaggacatagtgagaccctgtctcaaccaaacaacctggatcctttgcaagaagagcaggtgcttttaaacactgggccatctctccagcaccacctcccccctttaatgatacataggtcccacatagcctagttcggcctcttaactcctgacataacacctcatgaattcctcatcctcctgcctctaccttttgagtgagtgacgagattacaaacgttcgccaccatctttgttcccagttggccctcaaggccccagggtctcagttcagatcctgccactatggatacatagcattgaaatggggaaggctgcctctgtgctgcctcagcctttgggtgacgcctcttgcttggcactgcattttcaggagcatgacatctttgctctggaccctgaccccaaggttgtgtggagctgtaagaagtgtaagggtgtctggtattggtggcgcgcgcctttaatcccagcactcgggaggcagaggcagcagaggatctctgtgagttcaaggccagactagactacagagcgagttcaggacagccagggctgttaacacagagaaacactgtcttgaaaagcaaaacaaaataaaaccaacaaaacaacaacaacaacaaagtgttaagcgcgcatgcaaccagccactgggcaattagtcaaagatgccaagtctagatagacatgcagttaagaacttagggtctagagagatggctcggtggttaagagaccagaagagtttgcatcccagcgctcgcacagtagctaacaacagtctatcttacaccctcttccagcctctcctggcacaaggaacacacgtggtgctcatagttacaaaacagacacaacacgcatacacaaagaaataactaatttttaaaatgtcttgtaatcaagtaaaagtgctaactctagggactaaattaattcctcagtcgccctactccaggagcggtaaggctggccagaaagaccaagaacgcacgcgcggaggagaaaccacagagtcggtcctcccgggatagagaggccggaagtgctcgcggagctgcacgccgggtgctggaagcctactgagccccgaggaagggctccgctcggggcttggcgtggtgggtgagccggagggtcggcgtgagcggcctgggctttggttctgaatgatggcgtctcgggcaggcccgcgagcggccggcaccgacggcagcgactttcagcaccgggagcgcgtcgccatgcactaccagatgaggtatgaggtgagccaggagcactgaggccttccccgggaggagcctgcgggtctcgggaagcgacgcgggcgagcctcacggtgccgctcccccagccagctgtcgcgtactaccgggtccccggctccggcgagcgcctcgggtctgtttacaggccgggaagcccagtggcctgccctcgcccgcctcgtgctttgaggggatctggcctgcagaggctcaggggtcgatgctcagcccctctgaatgaccttggagacatcatttttcttttttcaaatcgaggtcccgcagtgtctagagttcaggctggcctggaactcacggccttccctcctcagcctcccgagtatgcgcctggtctgaaaactaacattcttaaagaaaactgcgtgtgtgcgtgtgtgtgtgtgtgtgtgtgtgtgtgtgcgcgcgcgcgcgcgcgtgtgtgtgtttgtgtgtgtgtaagtttgcccacacgagagcaggtgtgctggcagaatgcagtgccagtaaagggtgtgtgtgtgtgtgtgtgtgtgtgtgtgtgtgtgtgtgtgtgtgtgtgtgtgtgtgtgtgtgtgtgtgtgtgtgtgtgtgtgcgcgcgcgcgtgtgtgtgtttgtgtgtaagtttgcccacattcttaaagaaaactgcgtgtgtatgtgtgtgtgtgtgtgtgtgtgtgtgtgtgtgtgtgtgtttgtgtgtgtgtaagtttgcccacacgagagcaggtgtgctggcagaatgcagtgccagtaaagggtgttggatctcttggagctatagttacagacgtgtttaaatccacctgagactagtactgaggaccaaaccccgaagtgattttgtgagatagcggcttggtatggggtccatgctagcctccaggagcggcaggtgctcttaaccactgagccatctctccagtccccaggctgtcttggaacttgctctgtagaccagactagaaatcagagatctgcctgcctctgctgccaagtgcaaggattaaaggtgtacactacagccgccctgttgaccctgtctttaggaagatgtcatgtgtactttacaaagagttttgatgtagtcatgtcttaagctttgttgtcacacaagatgaaattaattaccaccagggaatttttcatcaaattgtgctgtacttaaatatgcctaaagtaaaatacttggggtcagactcttgaagtttaaaccaacaccaggtactgagttgtgttgaacccacacaactaccttgcctcctgcagattgttaactctgctggctgtggtggttgaagagacccccacattggcttagctgttaagagcacttgctgttcttggagagggtctaaactcagttcccagcaccgacatcaggaggctcacaaccaaacacctgcaactccggttccaggggttccgatgccctctggcttcccgacagcacaggcgtgtgcgtggtgcacacacatacacatgagataaatttttaaaaagaaagaaaagccaatctgggtcctgccttcaaggagcttgagtgccagaagtgattcttttctccttacctcccacatttcctataacaaattaattttccccttaaatccactgaattattcaactggttcttggcctctttctttacagaaggatgacaagccctttggacccttctttttcaactttaggcccctgcctaatggatgctcttgttttcatgcgtacctagagcagacaccttgcttgtgacaatatgaaaaggatataattgagaccccgagaagatcaggactaagagatgtgcctccttctcatggaaactatatctggagaaatacagggagcccaaactgtgttccttgttgctaggcctccccctaatggcctttgtttctgtgatacgaagccatctttctaaccacagctcagaattcttgtcatggcttggtgagataatgtcttagtacgttgtccaggccaacctccaatcagagatcctctgcctcagcctcccaggtgccattactgttcttcagtcttgaagtctacacatgcatggtctttgtaactgttactacctctctgtgccttggcagttgatttcctgaaacacaaatactaaaaagcttattcctttgtgcctgcatgtttttgcctctgcgtagaagcccacttccactcgtctcattacctctgctgaaatagctctctcctctagatataaccttctctggctttccatctcatgctaattggttcttccctgggatagcggatatcttcggtttatatgtatttacttgagttttatacttcatgtttcccagcctctatcagaggctgtgtcattcagattgtatctatgtgtccagtaccggtgcctggtggtcggagcaatttaaatgaaaattgcttctccctctgctctgccttacagtgtgacgctcaagagtgaaatcaagaagctgatctacgtacatctggtcatatggctgctgttggttgccaagatgtgtgtgggacacctgaggctcttgtcacatgaccaagtggctatgccctatcagtgggaatatccatatttattgagcattgtgccctctctcttgggccttctctccttccctcgaaacaacattagctacctggtgctctccatgatcagcatggggctcttctccatcgctcccctcatttatggcagcatggagatgttccctgccgcccagcaactctaccgccatggcaaggcctaccgcttcctgtttggtttctccgctgtctccgtcatgtacctagtgttgatactggcagtccaagttcatgcctggcaactgtactacagtaagaaactcttagactcttggttcaccagcacacaggagaagaaacgtaaatgaagcctgcctgatggacacatgaagggagctgttcagaatctccatggactgtggcatctgtgatgttggcacctagtgcacactatcctcagattttggccttgagttctctgttaccatctgctgagatgacaaatctgtagtgtttaatttattcttgactagccacaaaccggatgaccgatgtctgtggaacacttcaagttgaggccctccagactgagccttatccttgccttctcttggtcaaattccttacttccatttatcacctcttcatcaccgatactaaaagagatctggaataaatcagtgcagaaattctacttcaatctgtaggtcatggggcaagcaacatttgggaagttgctcccctaaaggctactctgtttactgcaaaccattttaattaaaaaagaacttcaataaagttaagactgtcctgtgctttgtgtttgagatttgattgaatttcaaagttgtattgctctagaggacttagacattatgagaagaatagatgtttcctgagaacatgggactggcgctggaggagagacagtgtgaagagtcttaagtcagctgcagccctctagctcttaggtgagaacaaccctgggatgggggttgctggaggaatggctcaggggttaagagcactgacagaggacacaggtttagttctcagcacccaaatggtgacttaaaaccatttgtaactactgtcccagggcatttgacgccttcttgcctccggatgcactaggcagacatgaggtacaaaaacaaacatgcaagcaaaacactcaaaaaattaaaaaaaaaaaacatggtctctcaatgtagctgtggatgtactagaactcactaagtagagcaatctgggctcaaactcacagagatcctcctgcctttgcctaccaagtacagggatttaaggtatgtgccaccacacttagcaatatatatttggccagacatttaatctccacccataaagcagcttatttggcttggattatgagctcccctttttatgcttgtttgagacagggcttctctatagctctggctgtcctggaacttgcttcgtggaccagactggcatcaaactcaaatccacctgcctctgcctcccacgtgctggcattaaaggtgtgtgcatcactgcccagctgggttacgagctttctatttggttatttatgtttattagacttttatgtgtctaaaactgagtaataatacccaaaaagaatctgcttgttggggctataattttccagtaactgacaaataaacctgagtcaaaataagaagaaaggggtttgagttctttttcccttttagtttctgtcattgattattccttggcctgcttgtttggggaatgtgcaaaggaagctgctaactctatggaagccagaaaaagaaaaaggtgagattccagtgtcgccactaggggcagactgccagtgacgtaacttcttcactcagcccctcccaaagcttcctccacctccaaaaatgtcaaggactggtaactagtacatgggcctttgagaaaagatagcagggagcttccagtcttgggggaatgtgcagtgaacataacatctaattgtgtgagagacccacagctgagatacagtttaagagtcacacctagaagtcttcacagtaaataaggcagcaaaggtgtgcatccctttgcactggactttattatgcctagatgatgctcagatctgtaactgcagagatctggccactacagttgtaattcttttgtcctggaaatgtgttgcttgtacagtctgcaaagctaattcctccagctatcccactgatcatttctttggagcaggggcagtttccagacagaatttctctgtgtagtcctcgctatcctggaactcagagatctgcctgctgtgtgctgggactaaaggcatgtaccaccatgcccagctcctactgatcatttcaaagataacttcttgtgcctcaggcctattgctgttcctgcttcctgtgtttacgggaacacagctctggatcatccaccctgcaagtttaagtccgtccccactccatatacacactgcctattcggtgaacctcctatagataccctgataagttccctagcttctcagtttaacacttagaactctccctacttaaggggaggcctctcagtggtgaagtgctcctctagcatgcaccgaggcctgggtgtgactccctcgtaacaacaaataaaaacaaaccttttctgctcttacattcctttattaatctttaagatgccacttgtatgaaaattcctcaattttcaattcctcaatttgaagaacaactttaatttgtctggttgctttcaccttattcctttattattattttctaacaagacagagcccagaatgaccttaagctatgtggctgaggatgactttgaactcctgatgctttgtctctacttcctaagtgctaggattacaagtatgcaccatcactcctggcttatattactcgtcccccacccccaccccatagaccaagccttgacctttatttttaaaagctgcatatttattctgctttctgacttagtgtttgtgccttcaacactttcacaacccttttctcttcctcaataaggaaagcctgcttgatcctgtcacggacacacttggcacacaaggagccaccacagcccctgctgacgtgcttctttgtctagacagtctcataaggactttgggtctcacagcatgaaccccttcaagtctgcctgggcacacaccacatggggattcaggtgctttcccagtcttcttggtataaaggtaaacaatcctgttgccaggggttcgagacagcctagtttcgttagaggctgtattgtaggaaagcctacaatggtatgtcaaacactggaccattcgagtgcctctaagcaccatccctggaagaggaagagctgtattatttgttttatattgacagaattgctactttgtatgtcttctgtagctcattagtgttcactctgagcttattgaatgaattaacttgcccttttgaggacaaaggtctgtatttcacagaaaacagaactggactgaagcagaaagaagcccatggacaagggagtttgtcatgagcttagctgaggcacacattgctaacttcaaacaactgtgatgcaaacaattaaaactgcagtgcagacccccctgctttctcccaataaattttccacataagttctgtcccccccccaaaaaaaaaacctgttagcacattattagtcaaagagctaaaacctgtttttggtcatcagaatagtgttttactgaaagtctttgaataccttgtagactaatttcactcattgtgaaattagtcaacagttttaaaacactatcatgccaacatcagtttttgttagttttgtgcttggaattctgcttttctagccttttcctataacttttttctgcctcatggttatggtgttcacaagtcctcttgatttagtgggaagaatgctatttgggttagaaagttcagctgggtatgggggtacatgtctaatcccaacctttggcaggttgaggcagcaggaatactctgagtttgaggccagcctgacatacatacatacatacatacatacatacatacatacatacatacaaacactatctcaaaaaacaaagttggagttaaagtatcaaccacaggactaagatcaacctgcctgttcacaggttgctttaaagctccaatagccggttggagagatggctcagaggttaagagcactggctgctcttccagaggtcctgagttcaattcccagcaaccacatagtggctcacagccatccattatgaggtcaggtgccctcttctggtgtgcagatgtacatggaagcagaatgttgttacataataaatacataaaatcttaaaaaaaaaaaactccaatagccaaagtattatgaaggcattctaaaatttttaatttattttcattttatttttggagacacagtttctctgtgtaaaaaccccaactgtcctggaactcactctagaccagactggcctccaactcacagagataaacctacctctgcctcccaagtactgggattaggccgggcagtggtatcgcccacctttaatcccaacactcgagaggcagaggttggtggatctctgtgagtttgagaccagcctggtctgcaagagctagttctaggacagcctccaaagccacagagaaaccctgtcttgaaaaaaacaaaaaacaaacaaaaagcaagttaatctggaattaaaagtgtatgccaccacacctgatgtaaatttaaaattttttttaaattaatttttctatacacaggtgttttgccagcatgtatgtttgtatagcacatgtgaacctggtgcctaaaaagccaaaagagtgcatctgatcccctgggactggagttacaaatggagtgctgccatgtgggtgctgagaattaaaaccaaatcttctagaagagcagccagtgagtgcccttaactgctgcagcatctttctggctcttgtgaaggcattttatggagtctgtaacaccatatgggtatcaaaagccgtgtggtcacctgatctttgcagcaagacaggagagagaaggtgtttgagagacttgagggatggtctagacaccaaagaaaggtttgtgtgttgtgagcaatgactgattattaggttgagattctagccagggagtttactatctcttgtttgatttattacattttattgccatcctaggaattaaatagggttttacatttgctaagcaagtgttctctcaacatccttatatcccctccccgatttctgacacttactatagctcagcttgtccttgaactcagcaatcctgcctccatagtgccgggattatggatgtttccactcccagctgagcctgttcttcatgtgttgcatgggcattgcagatctctcccattgctgtgaggctgaaataaatgtggatgaactttgtagtacatagtcgtttacatatcaagatgcctttccttggtaaacagtgttatgcttgcctctcaaattgggagtgtcttcctgttttaagaataatcctcttccctcttttcttcctctctcctcctttcccctccttctgacagggtctctttatagaatattcctgcctctctctgccttccaaatgttgtgtaggtgtacatcaccatgcctgcctagttagaatgacgtctcagaatgctggaggttaatttcacattcttagtccacattatcctgactggtatgttcaaggtgccctgacagtaggtacagacccagcagacctcaggaagtgaccattacagcaagatcctgtagctgctactcatagcctgttgggagcccgtgcataggaaagaatggcaagaaaaaatggctgccagagggcgtccccatagtatactctgtcactaagcatgcatgcttcagagcttgccaaaacctcctgagtccctggcttggtccccaaataaaggtcattagagcatgaagtcttggtcaactgattgagactcctttggagagtgcaaggcttctatgtagatgcccctgttgcctcctattctgtctaatttcttactctccctgttgacagctgccaatctcctttctcctaggactgtgcccactcatcactcactgtgataagacccctttccttttctttgttccttcctggtctgcctgcctgctcccccccccacctctcccttttcatggtctcatgaaaactgctctcagactcactatgtagctaaggatgactggagctcccaatcctcctgcccctacttccctaattctgggattacaagtttatgccaccacattccactttctgatttccttccttacatcttaattccactagctttatcacactgctaaagctaaaactttctttgcaaaatgagatccagaaacccacagactctccatagtaggttctgacagggaaacagctgacattgtatggtgttctgccttcccatttctttgctgtgtgtgtttttggacaagtctcggtcttcctgtatttccttccttccttccttccttccttccttctttttccattttttatttgaattataaacacgattgttttacatgttaatcccagttccctctccctcccctcctcccctaccaccatccccaactaaaaccctacctatcacatatcctttctgctccccagggagggtgaggccttccataggggtcctcagggtccgtcatatcctttgggatagggcctaggcccacctccgtgtatcttggctcagggagtatccctctatgtggaatgggctcccaaagtccacacctatgctaaggataagtactgctctactacaagaggctccatggatttctgaggtctcctcactaacacccacattcaggggtctggatcagttccatgctggtttcccagctatcagtctggggaccaagagctccctgttgttcaggtcagctgtttctgtgggtttcaccagcctggtctggacccctttgctcttcattcatccttctctgcaactgtattccagttcagtttagtgtttagctgtgggtgtctgcttctacttcttccagctgctggatgaaggctataggatggcatataagtcagtcatcaatgtcattatcaggggagggcatttaaagcagcctctcctctgttgcttagattgttagttggtgtcatctttgtagctctccaggcatttccctagtgcctgatttctctgtaaacctaaaattttccctctattatggtatctcttatcttgttttcttctattcttcccccaactcaacctttctgctccctcatatactcatcttcccttctcattctcctagctccttcctccccttcccaatttgctcaggagatctggtccctttccccttctccaggggaccatgtatgtctctcttagagtcctccttgttacctagcttctctggctttttttttttttttaagatttatttattatgtgtacagtgttctgcctgcaggtcagaagagggcaccagatcccattacagatggttgtgagccaccatgtggttgctgggaattgaactccggacctttggaagagcagtcagtgctcttaaccgctgagccatctctccagccctttctaaaactggacattagcctagcctcaagggttgtgatgctaacaatacaagctaaagagagcaatgggcatgatccaaagccagatagttcagcaaatattgatttcatcccttcccttttcggaacagactccagccacaccaaatatgtaaaccagcaagacaaaaaacagcaggctatcttttcccactttttgctttgtctatgtttgttggacaatgtcaaactatgtagcccagaccccttctgtaactttccatgtagaccaggctggcctccaactcaaattcccagcaatctacctgtctctgcctctggagttctgggaataaaggtgtgcatcaccatgcctggcctcaataagtccttgtcttatggtcttcttttctcctcctttacttcttatcctcctccctctctcttccctttccctttatctttatttctttattgtcaccgtttcttttctgcttctcttaccatgctttaacaaccatcaggaagcaactaaccaaatgttctcatcttgagagtcaggtcagaagatctaggagtcaagaaagacacgtgtaaaatagagctcacataccaagcattagaaaactcgcagtggcactaaaagatggctcaggggataagaacacttacatggtgactcacaaccatttgtaattccagttgcagacaattcaatatctccttctgatctctaagggcacccagaatgtacatacatacattcaggtaaaacgttcatatacattaaaaataataagccttaagaattagaaaagagggctggagagatggctcagaggttaagagcaccgactgctcttccagaggtcctgagttcaattcccagcaaccacatggtggctcacaaccatctgtaatgagatctggtgccctcttctggtgtgcagatatacatggaagctgaatgttgtatacatattaaataaataaaatcttaaaaaaagagaattagaaaagaaaaaaaagcttcatgtgtgtgtggaaacctccatgaggtagaatgggataaaagctcttgattctatgcttagtctggctagatcacctctcctgttctctggatctctctgacttgactgtagaggttggctctgtacaaggcagcactgtcagcagatcctgttggttcaaagaggtaggtgaagccaagagactacaggtgacagcagattgggagggcccagcacaggtgcagctcactggaagggtccagcacaggtgcagctcactggaagggcccagcacaagcgcagcaccttgggctatagactttgaccttgaccaaatcactttcctctctgcttccttatttctctccttacgaaattaaacaaataatcctcatttacctgcctcacaagttgtttagagggatcaaatattgctgttgctttgtgaactgtaagtagataagctatttagatgagatatatcatgattgacattcactcagccatgaagcacaggcttcctgacaggcactgctaggtgatggagaagaacaaatgagtctctccatctctcttttctcagcactaggattgaacccagggccttgcatacactaagcaagtgcactgccgccgagcttatctgtttttacctccttttaaaaatattgtttatgagctgggtggtggtggcacacttctttaatcccagcacttgagaggcagaggcagggggagctctgtgacttcgaggccagcctggtctgtagagtgagttccaagacagccagggctacaaaataaaaccctgtctcaaaaaacaaaaacaaaaacaaaaacaaaatcaacaaaatgtatgaatgcaaaaacaaaaggatactttagggtcgcaggtagattgggaacttcaaatgtgggccaaaagaaggctgtggccctgcttctgaaggatggtccagaccgaatctcagaaggacaaagtgtcggggctaagatgtagcttcgttgatagagttcatgcatgcctagcacgaaatcctgggttcagtctccagccctgaagaaactggatatggtggttcacgcccgtaacctcagcacttgggaggtagaggtagaaggaccaggagtttaaggaccgggcccagcctacgatacgtgagaacctgtcttgatgatgatgataatgggactggagagatggctcctgtgtaaagtgctgcataaacacgaggacctgagttctggtccccagcaccagcatgataccaggccatggcagaatatacctctagctccagtatcgagtagaggagacaggtggaccctggggcttgcctccccaccagcttagctgagacagcaaggtctaggttcaatgagagatcctgtctcaaaacaaaatagagataaatcaggaagacaccctggtgtcagctctggcctccacacgagcctgcaagcacacctgcccatacaacacacacacacacacacacacacacacacacacacacacacacacacacacacacacacacaccctggtgtcagctctggcctccacacgagcctgcaagcacacctgcccatacaacacacacacacacacacacacacacaaacacacacacacacacacacacacacacacacacctaaatttttttaattaatttaaaaagaagagacaatagcaataccaagatgcaatgacccagtggacatgcccctaactgtcctcccatcgtggagattgccaaggtgaggctgaagcagctaaagtctccatgaatgagaggaagctgtgagatgatggggagggtccccacaaagcctggagccttgtcagccttttttcctgcaccggaaaccacaggaaaccaggtcagtgtgtgtggctgctccctctgctccagagctcagctgtcagttttggggcttcctgagcaagctgtattatttagtttgtccttgaagctagagaccactttgacctggagtcgagaagagttggaaagccagctagctggggagccagagtgtacatggataacacaagaagtttatttgtgttatatgttggcctatttttttgcctaactttaaatgatgtaccaattgcttctttatcttcagcattcagtatagacaagtgccgaatagctgttaattctcaaaatggaagatgtgtaactgtctatgacagatccaagttctagaacatgctaataggcgtgccagatctgcagtgtttgaggagggttaagaaatgggtgcatttccacaacagggtgctggtccctttcctgttacctctccgccacaccaagagccccctgtgacgtgagagcagtaaggctttctaccaaacaggtggacaagtgaattagtgttcaccagtgccctggctcaggagaaggaatgtgacccagcctgctggttaggatagaggaggagcttcagggcagggtggatccagggcagggcttggtgagagcgcctgctggtactgtgtgctcctcacagctccagggccaggccctgctgccctgctgccctcctgcaaacaacaaagccatggtctctgtgccctgatcctgcagaagcttatggggagccccagactgacaacctggttcctgcctctcctcttactgctcttcagcctgtctatgtctgctgaggttggctgtccctacctgccacgctggaccagccactgtctactggcttcccatgtggtaaggtcctgctgctaggtgggggacacttgcttggcacagagtagaaactccccagtctcctgtctgccctttcctacctctgaaatccctgtgatctagtggcagcctctctgtgtccaccatgtggtcctgaaggttctgttgggctgaggtccttggaaggatgagaagaaaacagctggggtggatggacagtgggctgtgggtctcagaaccggatgatcggcacaaagagttcagggagcatgcacctcctgcagttcccttctaaccacacctctctcatttcttttccgcactgggctctgtacctccaggataagaattctgccggtgagtcctgtttcttgttctgcttcactccccatgctcccctgagttccaggtcccagtgagccatccatggccattttggttcccagcctggggagtagtctagtatcaatgggaaagtgcgggactagtgaagatttgcattgtaattctgcctcagacgctatgactgcttttcactctcccatctgaccctcggtctctgagcacaggagaagaggagaaatgattatgcgatgaggacagtgttgctctctaccaatggggaggataaaatgagcaccatggctaatctccaggcactctggacaagctggctcttcttatcaccggtacctcacttgaccaggaacaataatcatctttcatactttattgtgcattctgctttaccaagctttcccccatgccttatgtcacttggtccttatgacaaacccactttgagcagtctggcacctgcccaataacttaaccaagattgtacctgtaagctatatcagagcccagcccgaaatccagtttggagaaggtaggtgggaccccttaggccctaccatttttcttgttcctctttttgtttttgttttgagatagggcctcactatgtaataacaaagcctccataattatttatttacttattttggttcttcaagatagtgtttctctgtgtagtcctggctgacctggaactcactttgtagactaggctggccatgaactcacagagatccacctgcccctgcctcctgagtgctaggatcaaaggcatacgccatcaccacccagctgcctccatcttttaatccttccatctcagcctcccaaggaatagcttacagccatgtgttattgtgccgactttctatcatacacaggaagttctgttcaaactccctgccctaccctcctagccacgaagccttggcactggctctgctgtttgtgcctgcgtctgatgtcacggccttcaggtaagaaaagcagcctttgagccattcttagtgaaggcatgaatcagggacagttgaaaataactagctaggctgacacaagtgtcaattgtcatattgggacatatatttgtcttgttaacctcaagagggacttagagcatcctagaataacacatcatgacagacattcatgggaccactatatctttgtggaggtgccatctttcatctggcactaattcctaaaagccatgtgttctgtagacttgctattgttaccttccctctgtaaaatgggactgccactctgctgcttcattgcctcacttggtacatacggaacacacactttaaaaccagcagtcataggcggatggaaagatagcttagccatcaagagtactagatgctcttgcagaggacctaagttcaattcccagcacccatatcaggcagcatacaactgtaggtaactccaactctaaggaatctgatgtttctgatctcttaagcctcatttatatgtgccaatacatagatatacatacacatgcaagtaattaaacataaaataaaatcacaaaaagttagtttaaaattgttcaccattagctgagtgctagtggcgcacgcctttaatcccagcactcgggaggcagcggcaggaggatctctgtgattttgaggcccgcctgttctatagaatgagttctaggacaggctccaaaacaatacagagaaaccctgtctcaaataaataaataaataaataaataaataaataaataaacaaacaaactaagaaagaaatcatgctacaattaacactgcttttgtgggaggtgtgtgtgtttgtgtgtgtgtgcgtgtctgtcttttttctttgttttattttattttttaaatatcttatgttagaggtggagagatggctcagttgttgagacagttgctgcccttccagaggatctgtattagattcccagtaccctcatagtggctgtatacacaattgtctgtaactgtagttcagggaatccagtgccttcttttgacctcagcaggcaccaggcatgcgcatggtgtacattcatatgtgcaggtaaaaccttcatacacataaaataaaataaataaacctaaatatttaaaaaagaaatgaaatatcttttatttattgtttgacagtctcatacatgcagacaatctatcttggtcaaaaccacccacaattcctccctcccatgcacttggataactcccaatacatcactgctccacattcatgtctcttttaagatcttgtgtgtatgagtgctttgcctgcatgtatatatgtgtattacattcatgtagtactcatggaagcatcaaatccctgtagcactggagttatagatggttgggagccgccatgtgggtgctgggaaccaatcctgaatctaagaacatcaagtaggctgaactgctgatccatcttcacacacacacacacacacacacacacacacacacacacgcacgcacgcacgcacgcacgcacgcacgcacgcacacagagagccatcaaattttgagacagaggcttactgtgtagtcctggctcacctggaacttgcagagtagaccaggcaggtgtcaaaatcaagagatcagcctgcctctgcttctcaatacaagtataaaaccacgccttgctctgtttgttttgtaaattcacggagtccagctatgagtctgcctgctggaatgttgactgatctggttggcttgaccttgtgcagttcctgtgcaggtaaccacagctgcagtgggttcatgaacacgacagtcatgccatgtctagaagacttcaaaacactcttccacatcttctggctctcacattctttctgccctgtcttctgcatttgctgaaccttgaaggggcttaattctgttttttactgaatcataatttttgccaccaaagaagctcaatatcattgctacccttgatttctataaacattctacccaacattgggccacatctccagataggacaggggccaagaatgcccaggtaagaagtgtgtttccctcccacaggccttcagtggaactggtttcctctcttggtgaagaaatctaaaagtcctcctaagtctgaattctactggaggcgtagaaggccagcatcgtcccaggtaccctatcatttacaacactatggccctcagcccctttctttcttgccccttattttgttcttcagcaacaagagggagtttgaaaatgtggagattacgcactaagggaaactgaggccaaaggaatttgaacaacttgcctgttaaattaatttgtctctcagggttcagaggcagagttagaaagcacacttcctggatcctggcagacttcctggatcctggctcacatccactttgttctctgcttgcagaggaagctgctaagcagcctttccctgtctcaggaagaccatagcatttccaaccccttcccagccatctcccatggagggccacactgcaaaaggacccagcctttggacaccggaggagtagaacattttcctagagcaggttcacagaagcgtggaggtaggtgcgaggagcaacaagctggtggtctcagccttgcctgagccagtctctgagtcgtccttctccctaggacctgaattctcctttgatttgctgcctgaggcacaggctattcgggtgactattcctccaggcccagaggctagtgtgcgtctttgttaccagtgggcactggaatgtgaagacatgagcagtccttttgatacccaggtatggagttccatgccttgcaaagagacagattacacaatttctcagcaaatgggatggggtgggggggttggggcccagacaaggaaagtaattgtccaaaacttccttacaaaatgagataagtagcattaaaaacttgttgggggcgggggaggactagatagaaaagatggtgggctatgttacttgagaagagagaccagaaagaaatgagaatggtatgtattgccagggagaggactcttggtgtgattcgggtcaaattaagagactttgctaagggctggggacatacccgttggtagaatgtttacctagcatgcttgatctctggcactgcacagatggagtgggaggtggaagcaggagaatcagaagttcaaagtcttccttagatatataatgagtctgaggtctgtttggagtacgtgagtcttaagagaaaaaaaaaaaaaaagctttgttatttcctcaggaggttgttccctgaaaatttgacaaaactgtcatatgtgtcttggtacttttccctttcataccttttgtctagccctcagaaatgatattccccagtctcacaatccaggtcttgacagcctactttaactgccactcctcacagatgccattgtatgtagctgggcatcacatgcagagactggagtccttcagcttgcttcatgtctacaatggctataccttctaacaataccagcctttagtagaattgatgcttctaatactgcatggcatacagctggaactcaagaaatgtatcgcctcttccctaaccctactgcttgtagagtacctcaagaaaatggccaggatatagaacctatgagagggagaaataagttccatggagtaggaaagtaaggtatattgtccaaggtcccggcttcatcctgccttgtccatagtgtccatgcttttccttaggtgtctctttttctttccctttgtggttttatcaacagaaaattgtgtccgggggccacactgtagacctgccttatgaattcctcctgccctgcatgtgtatagaggtgagcacagggaaatgtggattagccatggctccttgtccaaccgcatctccagtaagtcaacttagaaaggactcgggaaggatggagatgacttggtgatagagcaggccgtacacactaaggagagactccaaaaaaaccgggctactgggatgcttaggaagctggtgctggctaggctatgagccacaccagaggtggaagggtcctaggcaattccacctcagccctgcctggatgctgtcttctctggtccagatttggcttcagctcacagacacagagtagacagccatcatcgctgtgttcttgagagccaaggttgcatatccctctcaaagtcatgttctttccaaccctccatttctccttgtaagtctgaagataaagatgttttggacatttttagggggaagacaggtactaaaagaagggtatggacagagacctgggaaacaagaagctgacagaggagacaaccagtggactggactcgtagggagtccagagtgaggagtcaacacagggtactgggtacttgagaaagtcaacaatggagctcagagagcctaaaattgagtgatgcaacctcgggaattctctgctgctgggaacagttctttggagacaggaggggtcatcccctctcatttgccactccctcctatgctctggaaccccaacacttacacacactccacagtgctgcccattattgtctgacagctcaagctgtttgatgacagtaagccagagccagaatgaagatgttaggaaagccatttctaacaaacactctctatgctccccaggcctcctacctgcaagaggacaccgtgagatgcaaaaagtgtcccttccagagctggcctgaagcctgtgagtgttgtttgtattaatcccgatgcacattcatactccccctgctgtgtgtgtgtgtgtgtgtgtgtgtgtgtgtgtgtgtgtgtgtgtgtgtgtgatggaggaagaccccagatggcaggaggacattttcttcaggagccaggggaagatgccacagctgtcagccaacagatgagctcctgagtgctgaacattatgaatggaaaaggatcgcccagcgagtgtgtgagagggccaactgtagacctaggctcacttctcagttctgccataggttatccccatagcccctcccctttccctcatctgtaaaatcccaaactgtttttgaggcttgaatgaaatattttaggggaaagtggctggtgttgtctggagtgctgtatgtagaatggtgaccttgaggagagaggacacttagacacaagtaacaaagtctaacgtacataggcagggttatcatcaagggtatcagcccaggacaggagaagcccagagacactgttgtgagcagagagggtgtcacaggatggcacttgactgagctcaggaactggacaggaattagaaagaagaagcagggggaagacagactctgtgaggagcctacacagattctggatacaggcgacagtaagggctgttcagtgtggaggcctggatggaaaggtaaagccaaggaagctgggtagaaagggagggagcgtggagtgcttgcctgttgaggacctgtgaagcctggaattgtgctggaacattggtattttctcctggtgtcctggggtggggtggggagatggagttacagctgggctatggcctcactcttgcacctgccacctgcccagatggctcggacttctggaagtcagtacacttcactgactacagccagaacaaccagatggtcatggctgtgacact

SEQ ID NO: 6 - DNA, 47 bp, Cricetulus griseus
  misc_feature 1..47: note = consensus sequence of group 1 ERVs
  source 1..47: mol_type = genomic DNA
gcccccgccatatccgccactgccgcccccaccagaggcagaagcgg

SEQ ID NO: 7 - DNA, 20 bp, Cricetulus griseus
  misc_feature 1..20: note = Myr2 5'-3 target site
  source 1..20: mol_type = genomic DNA
tcctaagcctagaaactatg

SEQ ID NO: 8 - DNA, 20 bp, Cricetulus griseus
  misc_feature 1..20: note = Myr4 5'-3 target site
  source 1..20: mol_type = genomic DNA
catagtttctaggcttagga

SEQ ID NO: 9 - DNA, 20 bp, Cricetulus griseus
  misc_feature 1..20: note = Myr8 5'-3 target site
  source 1..20: mol_type = genomic DNA
gagtgttagggacaaaggag

SEQ ID NO: 10 - DNA, 20 bp, Cricetulus griseus
  misc_feature 1..20: note = PPYP5 5'-3 target site
  source 1..20: mol_type = genomic DNA
gttggttgatctattaacgg

SEQ ID NO: 11 - DNA, 20 bp, Cricetulus griseus
  misc_feature 1..20: note = PYPP6 5'-3 target site
  source 1..20: mol_type = genomic DNA
gccactgccgcccccaccag

SEQ ID NO: 12 - DNA, 20 bp, Cricetulus griseus
  misc_feature 1..20: note = PPYP7 5'-3 target site
  source 1..20: mol_type = genomic DNA
gcccccaccagaggcagaag

SEQ ID NO: 13 - DNA, 20 bp, Cricetulus griseus
  misc_feature 1..20: note = PPYP13 5'-3 target site
  source 1..20: mol_type = genomic DNA
ggcagtggcggatatggcgg

SEQ ID NO: 14 - DNA, 20 bp, Cricetulus griseus
  misc_feature 1..20: note = PPYP20 5'-3 target site
  source 1..20: mol_type = genomic DNA
gcttctgcctctggtggggg

SEQ ID NO: 15 - DNA, 21 bp, synthetic construct
  misc_feature 1..21: note = modified Myr2 target sequence
  source 1..21: mol_type = other DNA
gtcctaagcctagaaactatg

SEQ ID NO: 16 - DNA, 27 bp, synthetic construct
  misc_feature 1..27: note = Myr2 primer 1
  source 1..27: mol_type = other DNA
acaccgtcctaagcctagaaactatgg

SEQ ID NO: 17 - DNA, 27 bp, synthetic construct
  misc_feature 1..27: note = Myr2 primer 2
  source 1..27: mol_type = other DNA
aaaaccatagtttctaggcttaggacg

SEQ ID NO: 18 - DNA, 21 bp, synthetic construct
  misc_feature 1..21: note = modified Myr4 5' 3' target sequence
  source 1..21: mol_type = other DNA
gcatagtttctaggcttagga

SEQ ID NO: 19 - DNA, 27 bp, synthetic construct
  misc_feature 1..27: note = Myr4 primer 1
  source 1..27: mol_type = other DNA
acaccgcatagtttctaggcttaggag

SEQ ID NO: 20 - DNA, 27 bp, synthetic construct
  misc_feature 1..27: note = Myr4 primer 2
  source 1..27: mol_type = other DNA
aaaactcctaagcctagaaactatgcg

SEQ ID NO: 21 - DNA, 26 bp, synthetic construct
  misc_feature 1..26: note = Myr8 primer 1
  source 1..26: mol_type = other DNA
acaccgagtgttagggacaaaggagg

SEQ ID NO: 22 - DNA, 26 bp, synthetic construct
  misc_feature 1..26: note = Myr8 primer 2
  source 1..26: mol_type = other DNA
aaaacctcctttgtccctaacactcg

SEQ ID NO: 23 - DNA, 26 bp, synthetic construct
  misc_feature 1..26: note = PPYP5 primer 1
  source 1..26: mol_type = other DNA
acaccgttggttgatctattaacggg

SEQ ID NO: 24 - DNA, 26 bp, synthetic construct
  misc_feature 1..26: note = PPYP5 primer 2
  source 1..26: mol_type = other DNA
aaaacccgttaatagatcaaccaacg

SEQ ID NO: 25 - DNA, 26 bp, synthetic construct
  misc_feature 1..26: note = PPYP6 primer 1
  source 1..26: mol_type = other DNA
acaccgccactgccgcccccaccagg

SEQ ID NO: 26 - DNA, 26 bp, synthetic construct
  misc_feature 1..26: note = PPYP6 primer 2
  source 1..26: mol_type = other DNA
aaaacctggtgggggcggcagtggcg

SEQ ID NO: 27 - DNA, 26 bp, synthetic construct
  misc_feature 1..26: note = PPYP7 primer 1
  source 1..26: mol_type = other DNA
acaccgcccccaccagaggcagaagg

SEQ ID NO: 28 - DNA, 26 bp, synthetic construct
  misc_feature 1..26: note = PPYP7 primer 2
  source 1..26: mol_type = other DNA
aaaaccttctgcctctggtgggggcg

SEQ ID NO: 29 - DNA, 26 bp, synthetic construct
  misc_feature 1..26: note = PPYP13 primer 1
  source 1..26: mol_type = other DNA
acaccggcagtggcggatatggcggg

SEQ ID NO: 30 - DNA, 26 bp, synthetic construct
  misc_feature 1..26: note = PPYP13 primer 2
  source 1..26: mol_type = other DNA
aaaacccgccatatccgccactgccg

SEQ ID NO: 31 - DNA, 26 bp, synthetic construct
  misc_feature 1..26: note = PPYP20 primer 1
  source 1..26: mol_type = other DNA
acaccgcttctgcctctggtgggggg

SEQ ID NO: 32 - DNA, 26 bp, synthetic construct
  misc_feature 1..26: note = PPYP20 primer 2
  source 1..26: mol_type = other DNA
aaaaccccccaccagaggcagaagcg

SEQ ID NO: 33 - DNA, 55 bp, synthetic construct
  misc_feature 1..55: note = Myr_Fa_3 forward primer
  source 1..55: mol_type = other DNA
tcgtcggcagcgtcagatgtgtataagagacagaccgcttgaaggatttgcaatc

SEQ ID NO: 34 - DNA, 52 bp, synthetic construct
  misc_feature 1..52: note = Myr_Fb_0 forward primer
  source 1..52: mol_type = other DNA
tcgtcggcagcgtcagatgtgtataagagacaggcttgagggatttgcaatc

SEQ ID NO: 35 - DNA, 53 bp, synthetic construct
  misc_feature 1..53: note = Myr_Fb_1 forward primer
  source 1..53: mol_type = other DNA
tcgtcggcagcgtcagatgtgtataagagacagtgcttgagggatttgcaatc

SEQ ID NO: 36 - DNA, 54 bp, synthetic construct
  misc_feature 1..54: note = Myr_Fb_2 forward primer
  source 1..54: mol_type = other DNA
tcgtcggcagcgtcagatgtgtataagagacagttgcttgagggatttgcaatc

SEQ ID NO: 37 - DNA, 55 bp, synthetic construct
  misc_feature 1..55: note = Myr_Fb_3 forward primer
  source 1..55: mol_type = other DNA
tcgtcggcagcgtcagatgtgtataagagacagactgcttgagggatttgcaatc

SEQ ID NO: 38 - DNA, 54 bp, synthetic construct
  misc_feature 1..54: note = Myr_Fc_2 forward primer
  source 1..54: mol_type = other DNA
tcgtcggcagcgtcagatgtgtataagagacaggtgcttgagggatttgtaatc

SEQ ID NO: 39 - DNA, 55 bp, synthetic construct
  misc_feature 1..55: note = Myr_R_0 reverse primer
  source 1..55: mol_type = other DNA
gtctcgtgggctcggagatgtgtataagagacagacaaagagtaatccatttgcg

SEQ ID NO: 40 - DNA, 56 bp, synthetic construct
  misc_feature 1..56: note = Myr_R_1 reverse primer
  source 1..56: mol_type = other DNA
gtctcgtgggctcggagatgtgtataagagacaggacaaagagtaatccatttgcg

SEQ ID NO: 41 - DNA, 57 bp, synthetic construct
  misc_feature 1..57: note = Myr_R_2 reverse primer
  source 1..57: mol_type = other DNA
gtctcgtgggctcggagatgtgtataagagacagcgacaaagagtaatccatttgcg

SEQ ID NO: 42 - DNA, 58 bp, synthetic construct
  misc_feature 1..58: note = Myr_R_3 reverse primer
  source 1..58: mol_type = other DNA
gtctcgtgggctcggagatgtgtataagagacagaagacaaagagtaatccatttgcg

SEQ ID NO: 43 - DNA, 52 bp, synthetic construct
  misc_feature 1..52: note = PPYP_Fa_0 forward primer
  source 1..52: mol_type = other DNA
tcgtcggcagcgtcagatgtgtataagagacagactccagcctttaccctac

SEQ ID NO: 44 - DNA, 53 bp, synthetic construct
  misc_feature 1..53: note = PPYP_Fa_1 forward primer
  source 1..53: mol_type = other DNA
tcgtcggcagcgtcagatgtgtataagagacagaactccagcctttaccctac

SEQ ID NO: 45 - DNA, 52 bp, synthetic construct
  misc_feature 1..52: note = PPYP_Fb_0 forward primer
  source 1..52: mol_type = other DNA
tcgtcggcagcgtcagatgtgtataagagacagattccaacctttaccctac

SEQ ID NO: 46 - DNA, 53 bp, synthetic construct
  misc_feature 1..53: note = PPYP_Fb_1 forward primer
  source 1..53: mol_type = other DNA
tcgtcggcagcgtcagatgtgtataagagacaggattccaacctttaccctac

SEQ ID NO: 47 - DNA, 54 bp, synthetic construct
  misc_feature 1..54: note = PPYP_Fb_2 forward primer
  source 1..54: mol_type = other DNA
tcgtcggcagcgtcagatgtgtataagagacagtgattccaacctttaccctac

SEQ ID NO: 48 - DNA, 55 bp, synthetic construct
  misc_feature 1..55: note = PPYP_Fb_3 forward primer
  source 1..55: mol_type = other DNA
tcgtcggcagcgtcagatgtgtataagagacagcttattccaacctttaccctac

SEQ ID NO: 49 - DNA, 53 bp, synthetic construct
  misc_feature 1..53: note = PPYP_Ra_1 reverse primer
  source 1..53: mol_type = other DNA
gtctcgtgggctcggagatgtgtataagagacaggggtctgatgctgagaatg

SEQ ID NO: 50 - DNA, 52 bp, synthetic construct
  misc_feature 1..52: note = PPYP_Rb_0 reverse primer
  source 1..52: mol_type = other DNA
gtctcgtgggctcggagatgtgtataagagacagggtccgatgctgagaatg

SEQ ID NO: 51 - DNA, 53 bp, synthetic construct
  misc_feature 1..53: note = PPYP_Rb_1 reverse primer
  source 1..53: mol_type = other DNA
gtctcgtgggctcggagatgtgtataagagacagtggtccgatgctgagaatg

SEQ ID NO: 52 - DNA, 54 bp, synthetic construct
  misc_feature 1..54: note = PPYP_Rb_2 reverse primer
  source 1..54: mol_type = other DNA
gtctcgtgggctcggagatgtgtataagagacaggtggtccgatgctgagaatg

SEQ ID NO: 53 - DNA, 55 bp, synthetic construct
  misc_feature 1..55: note = PPYP_Rb_3 reverse primer
  source 1..55: mol_type = other DNA
gtctcgtgggctcggagatgtgtataagagacagaagggtccgatgctgagaatg

SEQ ID NO: 54 - DNA, 23 bp, synthetic construct
  misc_feature 1..23: note = PCR ERV Type 1 validation primer
  source 1..23: mol_type = other DNA
ctctggttcttgcctgctgagct

SEQ ID NO: 55 - DNA, 22 bp, synthetic construct
  misc_feature 1..22: note = PCR ERV Type 1 validation primer
  source 1..22: mol_type = other DNA
tggtcaatgtatatgaggcgct

SEQ ID NO: 56 - DNA, 22 bp, synthetic construct
  misc_feature 1..22: note = qPCR Type1 ERV specific LTR primer
  source 1..22: mol_type = other DNA
gggaattgagtctgctgtacca

SEQ ID NO: 57 - DNA, 22 bp, synthetic construct
  misc_feature 1..22: note = qPCR Type1 ERV specific LTR primer
  source 1..22: mol_type = other DNA
acagagtctttcaaatgaggcg

SEQ ID NO: 58 - DNA, 22 bp, synthetic construct
  misc_feature 1..22: note = qPCR reference GAPDH primer 1
  source 1..22: mol_type = other DNA
gcgacttcaacagtgactccca

SEQ ID NO: 59 - DNA, 22 bp, synthetic construct
  misc_feature 1..22: note = qPCR reference GAPDH primer 2
  source 1..22: mol_type = other DNA
tgaggtccaccactctgttgct

SEQ ID NO: 60 - DNA, 22 bp, synthetic construct
  misc_feature 1..22: note = qPCR Type 2 ERV specific primer 1
  source 1..22: mol_type = other DNA
gaataaaaggtcagggcgttgg

SEQ ID NO: 61 - DNA, 22 bp, synthetic construct
  misc_feature 1..22: note = qPCR Type 2 ERV specific primer 2
  source 1..22: mol_type = other DNA
ctgacttggctctatcttgggt

SEQ ID NO: 62 - DNA, 20 bp, synthetic construct
  misc_feature 1..20: note = qPCR Type 1 ERV specific Gag primer 1
  source 1..20: mol_type = other DNA
tgacgatataagccacttga

SEQ ID NO: 63 - DNA, 23 bp, synthetic construct
  misc_feature 1..23: note = qPCR Type 1 ERV specific Gag primer 2
  source 1..23: mol_type = other DNA
acccccagactatattccagata

SEQ ID NO: 64 - DNA, 20 bp, synthetic construct
  misc_feature 1..20: note = qPCR Type1 ERV specific Env primer 1
  source 1..20: mol_type = other DNA
ctatgtgctgccctcaagga

SEQ ID NO: 65 - DNA, 20 bp, synthetic construct
  misc_feature 1..20: note = qPCR Type1 ERV specific Env primer 2
  source 1..20: mol_type = other DNA
gcctctccctaagtttggcc

SEQ ID NO: 66 - DNA, 21 bp, synthetic construct
  misc_feature 1..21: note = PCR locus ERV Type 1 validation 2 primer
  source 1..21: mol_type = other DNA
taagccattggtgaagggtca

SEQ ID NO: 67 - DNA, 23 bp, synthetic construct
  misc_feature 1..23: note = PCR locus ERV Type 1 validation 4 primer
  source 1..23: mol_type = other DNA
ttttctggtgccctcttgcctgg

SEQ ID NO: 68 - DNA, 23 bp, synthetic construct
  misc_feature 1..23: note = PCR locus ERV Type 1 validation primer without ERV
  source 1..23: mol_type = other DNA
ttgtggagctgtgtgagtggtgg

SEQ ID NO: 69 - DNA, 63 bp, Cricetulus griseus
  misc_feature 1..2: note = genomic Myr2 sgRNA target sequence
  source 1..63: mol_type = genomic DNA
ctgtcatttgtgccctcctaagcctagaaactatggggcaaactgtcaccactcctttgtccc

SEQ ID NO: 70 - DNA, 64 bp, synthetic construct
  misc_feature 1..64: note = cloned sequence of Myr2 mutated mRNA juction
  source 1..64: mol_type = other DNA
ctgtcatttgtgccctcctaagcctagaaacttatggggcaaactgtcaccactcctttgtccc

SEQ ID NO: 71 - DNA, 51 bp, synthetic construct
  misc_feature 1..51: note = cloned sequence of Myr2 mutated mRNA junction
  source 1..51: mol_type = other DNA
ctgtcatttgtgccctcctaagcctagaaactgtcaccactcctttgtccc

SEQ ID NO: 72 - DNA, 222 bp, Cricetulus griseus
  misc_feature 1..222: note = genomic PPYP6 sgRNA target sequence
  source 1..222: mol_type = genomic DNA
cctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccatgccgcccccaccagaggcagaagcggactccgccgctgccttggcggaagcggcccctgaccctt

SEQ ID NO: 73 - DNA, 168 bp, synthetic construct
  misc_feature 1..168: note = cloned sequence of PPYP6 mutated mRNA junction
  source 1..168: mol_type = other DNA
cctttgattcctcccaaccccccttcccattccaacctttaccctaccgtgatgaaagacactaaggctaaagaaaagaagacacctaaggtactccctccgggagaagaccccccgccatatccgccatatccgccgctgccttggcggaagcggcccctgaccctt

SEQ ID NO: 74 - DNA, 64 bp, Cricetulus griseus
  misc_feature 1..64: note = genomic PPYP6 sgRNA target sequence
  source 1..64: mol_type = genomic DNA
gcccccgccatatccgccactgccgcccccaccagaggcagaagcggactccgccgctgccttg

SEQ ID NO: 75 - DNA, 65 bp, synthetic construct
  misc_feature 1..65: note = cloned sequence of PPYP6 mutated mRNA junction
  source 1..65: mol_type = other DNA
gcccccgccatatccgccactgccgcccccacccagaggcagaagcggactccgccgctgccttg

SEQ ID NO: 76 - DNA, 117 bp, Cricetulus griseus
  misc_feature 1..117: note = genomic PPYP6 sgRNA target sequence
  source 1..117: mol_type = genomic DNA
tccgggagaagaccagttggttgatctattaacggaggagcccccgccatatccgccactgccgcccccaccagaggcagaagcggactccgccgctgccttggcggaagcggcccc

SEQ ID NO: 77 - DNA, 113 bp, synthetic construct
  misc_feature 1..113: note = cloned sequence of PPYP6 mutated mRNA junction
  source 1..113: mol_type = other DNA
tccgggagaagaccagttggttgatctattaacggaggagcccccgccatatccgccactgccgccccagaggcagaagcggactccgccgctgccttggcggaagcggcccc

SEQ ID NO: 78 - DNA, 382 bp, Cricetulus griseus
  misc_feature 1..382: note = genomic PPYP6 sgRNA target sequence
  source 1..382: mol_type = genomic DNA
atggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaccagaggcagaagcggactccgccgctgccttggcgg

SEQ ID NO: 79 - DNA, 399 bp, synthetic construct
  misc_feature 1..399: note = cloned sequence of PPYP6 mutated mRNA junction
  source 1..399: mol_type = other DNA
atggatcctggaccacacgggcatcccgatcaagtggcttatatcgtcacttgggaggctttggttcaggacccccctccctgggtacgtcctttcttacatcccaagggcccctctctccttcccccctctaaccgctccaaccgacccattccttcggcccctacacctcccactcctttgattcctcccaaccccccttcccattccaacctttaccctaccgtgatgaaagacactaaggctaaagaaaagaagacacctaaggtactccctccgggagaagaccagttggttgatctattaacggaggagcccccgccatatccgccactgccgcccccaagtgacgatataagccacttgatcgggatgcggactccgccgctgccttggcgg

SEQ ID NO: 80 - DNA, 110 bp, Cricetulus griseus
  misc_feature 1..110: note = genomic PPYP13 sgRNA target sequence
  source 1..110: mol_type = genomic DNA
ttaacggaggagcccccgccatatccgccactgccgcccccaccagaggcagaagcggactccgccgctgccttggcggaagcggcccctgacccttcaccaatggctta

SEQ ID NO: 81 - DNA, 122 bp, synthetic construct
  misc_feature 1..122: note = cloned sequence of PPYP13 mutated mRNA junction
  source 1..122: mol_type = other DNA
ttaacggaggagcccccgccatatccgccactgccgcccccaccagaggcagaagcggactccgccgctgccttggcggaagcggccccagatccaccacctgacccttcaccaatggctta

SEQ ID NO: 82 - DNA, 81 bp, Cricetulus griseus
  misc_feature 1..81: note = genomic Myr2 sgRNA target sequence
  source 1..81: mol_type = genomic DNA
tgtcatttgtgccctcctaagcctagaaactatggggcaaactgtcaccactcctttgtccctaacactctcccactggaa

SEQ ID NO: 83 - DNA, 79 bp, synthetic construct
  misc_feature 1..79: note = cloned sequence of Myr2 mutated mRNA junction
  source 1..79: mol_type = other DNA
tgtcatttgtgccctcctaagcctagaaactggggcaaactgtcaccactcctttgtccctaacactctcccactggaa

SEQ ID NO: 84 - DNA, 53 bp, Cricetulus griseus
  misc_feature 1..53: note = 5' and 3' end of genomic Myr2 sgRNA target sequence
  misc_feature 18..19: note = site of 75 bp additon in Myr2 sgRNA target sequence
  source 1..53: mol_type = genomic DNA
cgactctctctcaattcctgaaactatggggcaaactgtcaccactcctttgt

SEQ ID NO: 85 - DNA, 33 bp, synthetic construct
  misc_feature 1..33: note = cloned sequence of Myr2 mutated mRNA junction
  source 1..33: mol_type = other DNA
cgactctctctcaaactgtcaccactcctttgt

SEQ ID NO: 86 - DNA, 127 bp, Cricetulus griseus
  misc_feature 1..127: note = genomic Myr2 sgRNA target sequence
  source 1..127: mol_type = genomic DNA
tctttgtcttgtagctgtcatttgtgccctcctaagcctagaaactatggggcaaactgtcaccactcctttgtccctaacactctcccactggaaagatgtacaggaatatgctcataaccaatct

SEQ ID NO: 87 - DNA, 100 bp, synthetic construct
  misc_feature 1..100: note = cloned sequence of Myr2 mutated mRNA junction
  source 1..100: mol_type = other DNA
tctttgtcttgtagctgtcatggggcaaactgtcaccactcctttgtccctaacactctcccactggaaagatgtacaggaatatgctcataaccaatct

SEQ ID NO: 88 - DNA, 65 bp, Cricetulus griseus
  misc_feature 1..65: note = genomic Myr2 sgRNA target sequence
  source 1..65: mol_type = genomic DNA
agctgtcatttgtgccctcctaagcctagaaactatggggcaaactgtcaccactcctttgtccc

SEQ ID NO: 89 - DNA, 62 bp, synthetic construct
  misc_feature 1..62: note = cloned sequence of Myr2 mutated mRNA junction
  source 1..62: mol_type = other DNA
agctgtcatttgtgccctcctaagcctagatatggggcaaactgtcaccactcctttgtccc

SEQ ID NO: 90 - DNA, 41 bp, Cricetulus griseus
  misc_feature 1..41: note = genomic Myr2 sgRNA target sequence
  source 1..41: mol_type = genomic DNA
ctcctaagcctagaaactatggggcaaactgtcaccactcc

SEQ ID NO: 91 - DNA, 42 bp, synthetic construct
  misc_feature 1..42: note = cloned sequence of Myr2 mutated mRNA junction
  source 1..42: mol_type = other DNA
ctcctaagcctagaaacttatggggcaaactgtcaccactcc

SEQ ID NO: 92 - DNA, 71 bp, Cricetulus griseus
  misc_feature 1..71: note = genomic PPYP6 sgRNA target sequence
  source 1..71: mol_type = genomic DNA
cccccgccatatccgccactgccgcccccaccagaggcagaagcggactccgccgctgccttggcggaagc

SEQ ID NO: 93 - DNA, 61 bp, synthetic construct
  misc_feature 1..61: note = cloned sequence of PPYP6 mutated mRNA junction
  source 1..61: mol_type = other DNA
cccccgccatatccgccactgccgccccactgcttctgtccgccgctgccttggcggaagc

SEQ ID NO: 94 - DNA, 65 bp, Cricetulus griseus
  misc_feature 1..65: note = genomic PPYP6 sgRNA target sequence
  source 1..65: mol_type = genomic DNA
cccccgccatatccgccactgccgcccccaccagaggcagaagcggactccgccgctgccttggc

SEQ ID NO: 95 - DNA, 58 bp, synthetic construct
  misc_feature 1..58: note = cloned sequence of PPYP6 mutated mRNA junction
  source 1..58: mol_type = other DNA
cccccgccatatccgccactgccgcccccacagaagcggactccgccgctgccttggc

SEQ ID NO: 96 - DNA, 66 bp, synthetic construct
  misc_feature 1..66: note = cloned sequence of PPYP6 mutated mRNA junction
  source 1..66: mol_type = other DNA
cccccgccatatccgccactgccgcccccacccagaggcagaagcggactccgccgctgccttggc

SEQ ID NO: 97 - DNA, 91 bp, Cricetulus griseus
  misc_feature 1..91: note = genomic PPYP6 sgRNA target sequence
  source 1..91: mol_type = genomic DNA
ggagaagaccagttggttgatctattaacggaggagccccccgccatatccgccactgccgcccccaccagaggcagaagcggactccgcc

SEQ ID NO: 98 - DNA, 82 bp, synthetic construct
  misc_feature 1..82: note = cloned sequence of PPYP6 mutated mRNA junction
  source 1..82: mol_type = other DNA
ggagaagaccagttggttgatctattaacggaggagccccccgccatatccgccactgccgcccccagaagcggactccgcc

SEQ ID NO: 99 - DNA, 65 bp, Cricetulus griseus
  misc_feature 1..65: note = genomic PPYP6 sgRNA target sequence
  source 1..65: mol_type = genomic DNA
agcccccgccatatccgccactgccgcccccaccagaggcagaagcggactccgccgctgccttg

SEQ ID NO: 100 - DNA, 28 bp, synthetic construct
  misc_feature 1..28: note = cloned sequence of PPYP6 mutated mRNA junction
  source 1..28: mol_type = other DNA
agcccccgccatatccgccgctgccttg

SEQ ID NO: 101 - DNA, 65 bp, Cricetulus griseus; identical to SEQ ID NO: 94
  misc_feature 1..65: note = genomic PPYP6 sgRNA target sequence
  source 1..65: mol_type = genomic DNA

SEQ ID NO: 102 - DNA, 62 bp, synthetic construct
  misc_feature 1..62: note = cloned sequence of PPYP6 mutated mRNA junction
  source 1..62: mol_type = other DNA
cccccgccatatccgccactgccgcccccagaggcagaagcggactccgccgctgccttggc

SEQ ID NO: 103 - DNA, 73 bp, Cricetulus griseus
  misc_feature 1..73: note = genomic PPYP6 sgRNA target sequence
  source 1..73: mol_type = genomic DNA
cccccgccatatccgccactgccgcccccaccagaggcagaagcggactccgccgctgccttggcggaagcgg

SEQ ID NO: 104 - DNA, 51 bp, synthetic construct
  misc_feature 1..51: note = cloned sequence of PPYP6 mutated mRNA junction
  source 1..51: mol_type = other DNA
cccccgccatatccgccactgcggactccgccgctgccttggcggaagcgg

SEQ ID NO: 105 - DNA, 60 bp, Cricetulus griseus
  misc_feature 1..60: note = genomic PPYP6 sgRNA target sequence
  source 1..60: mol_type = genomic DNA
cccccgccatatccgccactgccgcccccaccagaggcagaagcggactccgccgctgcc

SEQ ID NO: 106 - DNA, 59 bp, synthetic construct
  misc_feature 1..59: note = cloned sequence of PPYP6 mutated mRNA junction
  source 1..59: mol_type = other DNA
cccccgccatatccgccactgccgcccccacagaggcagaagcggactccgccgctgcc

SEQ ID NO: 107 - DNA, 65 bp, Cricetulus griseus
  misc_feature 1..65: note = genomic PPYP6 sgRNA target sequence
  source 1..65: mol_type = genomic DNA
ttaacggaggagcccccgccatatccgccactgccgcccccaccagaggcagaagcggactccgc

SEQ ID NO: 108 - DNA, 63 bp, synthetic construct
  misc_feature 1..63: note = cloned sequence of PPYP6 mutated mRNA junction
  source 1..63: mol_type = other DNA
ttaacggaggagcccccgccatatccgccactgccgcccccacaaggcagaagcggactccgc

SEQ ID NO: 109 - DNA, 90 bp, Cricetulus griseus
  misc_feature 1..90: note = genomic PPYP6 sgRNA target sequence
  source 1..90: mol_type = genomic DNA
aggagcccccgccatatccgccactgccgcccccaccagaggcagaagcggactcccccgccatatccgcggaagcggcccctgaccctt

SEQ ID NO: 110 - DNA, 77 bp, synthetic construct
  misc_feature 1..77: note = cloned sequence of PPYP6 mutated mRNA junction
  source 1..77: mol_type = other DNA
aggagcccccgccatatccgccactgccgcagaagcggactccgccgctgccttggcggaagcggcccctgaccctt

(19) United States
(12) Patent Application Publication
(10) Pub. No.: US 2025/0346928 A1
(43) Pub. Date: Nov. 13, 2025

(54) CHARACTERIZATION AND INACTIVATION OF ENDOGENOUS RETROVIRUSES IN CHINESE HAMSTER OVARY CELLS

(71) Applicant: Selexis S.A., Plan-les-Ouates (CH)

(72) Inventors: Pierre-Olivier Duroy, Plan-Les-Ouates (CH); Sandra Bosshard, Plan-Les-Ouates (CH); Philippe Le Mercier, Plan-Les-Ouates (CH); Emanuel Schmid-Siegert, Plan-Les-Ouates (CH); Nicolas Mermod, Plan-Les-Ouates (CH)

(73) Assignee: Selexis S.A., Plan-les-Ouates (CH)

(21) Appl. No.: 19/287,883

(22) Filed: Aug. 1, 2025

Related U.S. Application Data
(63) Continuation of application No. 17/417,131, filed on Jun. 22, 2021, now Pat. No. 12,404,527.
(60) Provisional application No. 62/784,566, filed on Dec. 24, 2018.

(51) Int. Cl. C12N 15/90; C07K 14/005; C12N 15/67

(52) U.S. Cl. CPC C12N 15/907; C07K 14/005; C12N 15/67; C12N 2740/10023; C12N 2740/13021; C12N 2740/13022

(57) ABSTRACT

Type-C endogenous retroviruses (ERVs) embedded in Chinese hamster ovary (CHO) cells were altered to modify the release of retroviral and/or retroviral-like particles in the culture supernatant. Although evidence for the infectivity of these particles is missing. their presence has raised safety concerns. 173 type-C ERV sequences that clustered into functionally conserved groups were identified. Transcripts from one type-C ERV group were identified to be full-length with intact open reading frames, and to have corresponding viral RNA genomes that were loaded into retroviral-like particles. Also, sequence analysis of the genomic RNA from viral particles indicated that they may result from few expressed ERV sequences. Disclosed herein is the disruption/alteration of the gag gene of the expressed ERV group using CRISPR-Cas9 genome editing. Comparison of CRISPR-derived mutations at the DNA and mRNA level led to the identification of a single ERV locus responsible for the release of viral RNA-loaded particles from CHO cells. Clones bearing a Gag loss-of-function mutation in this particular ERV locus showed a reduction of viral RNA-containing particles in the cell supernatant by over 250-fold. Notably, ERV mutagenesis did not compromise cell growth, cell size or recombinant protein production. Provided herein is a new strategy and cells, in particular engineered CHO cells, to mitigate potential contaminations from CHO endogenous retroviruses during biopharmaceutical manufacturing.

### CROSS REFERENCE TO RELATED APPLICATIONS

This application is a divisional application of U.S. patent application Ser. No. 17/417,131, which is the U.S. national stage of international patent application no. PCT/EP2019/086873, filed Dec. 20, 2019 designating the United States and claiming priority to U.S. provisional application No. 62/784,566, filed Dec. 24, 2018, which are incorporated herein by reference in their entireties.

### REFERENCE TO AND INCORPORATION OF ELECTRONIC SEQUENCE LISTING

This application contains a Sequence Listing which has been submitted electronically in .XML format and is hereby incorporated by reference in its entirety. Said .XML copy, created on Jul. 29, 2025, is named “3024-278NSDIV.xml” and is 336,122 bytes in size. The sequence listing contained in this .XML file is part of the specification and is hereby incorporated by reference herein in its entirety.

### BACKGROUND AND INTRODUCTION

Contamination of biopharmaceutical products by adventitious agents such as viruses can interrupt drug supply and thereby imperil patient safety. Although viral contaminations of biopharmaceuticals are rare, they still occur (1), and mitigating the risk of viral contaminations in therapeutic protein preparations remains a top priority.

Chinese hamster ovary (CHO) cells are the most widely used mammalian expression system for biopharmaceutical products. Among others, CHO cells became a preferred production host in view of their superior safety profile compared to other cell lines used for recombinant protein production. For instance, it was shown that CHO cells possess reduced susceptibility to certain viral infections (1), including resistance to infections elicited by many human as well as murine retroviruses, with some of the latter being known to infect other mammalian cells (2, 3). In addition, CHO cells, unlike other rodent cells, appeared to be unable to produce infective retroviruses that could replicate in mammalian cells, notably in human cells (3-6). However, viral-like particles (VLPs) have been detected both within CHO cells as well as budding off in the culture medium (7-11). The presence of such VLPs raises safety and regulatory concerns, not only because there is a remaining risk of a possible hamster to human endogenous retrovirus (ERV) transmission, but also because they interfere with and reduce the sensitivity of the detection of other adventitious agents.

The publications and other materials, including patents and patent applications, used herein to illustrate the invention and, in particular, to provide additional details respecting the practice the invention are incorporated herein by reference in their entirety. For convenience, the publications are referenced in the following text either by a number for reference to the appended bibliography, by the name of the authors and year published or by the patent/patent publication number.

VLPs were detected independently by several laboratories, suggesting that they result from ERVs that stably integrated into the CHO genome, rather than from an exogenous infection (12). CHO cells possess two classes of ERVs: the intracisternal type-A ERVs (IAP), a defective ERV class forming immature particles in the cisternae of the endoplasmic reticulum (13), and the budding type-C ERVs (6, 12). Although type-C ERV sequences remain incompletely characterized, a previous study estimated that approximately 100-300 type-C ERV sequences may be present in the CHO genome (6). Some of them seemed to be full-length and actively transcribed proviruses, such as the ML2G retrovirus (10, 12). However, the ML2G ERV sequences described by Lie et al., contain frameshift mutations in each of its gene (Gag, Pol and Env), indicating that the specific ERV sequence at this locus is not producing any VLP (12). Nevertheless, this publication indicated that other members of this type of ERV sequence are transcribed and may produce VLP. The ML2G transcript shares approximately 64% sequence identity to the murine leukemia virus (MLV) family.

CHO cells are generally believed to produce non-infective retroviral particles, as their infectivity could not be demonstrated. Nevertheless, the risk that at least one of the uncountable predicted type-C ERV proviruses in the CHO genome is or becomes infective cannot be excluded. This may happen if epigenetically silenced ERVs become expressed, as was observed upon some chemical treatments (14), if dysfunctional ERVs may acquire gain-of-function mutations or if ERVs recombine or trans complement each other. Such genetic changes are more likely to occur in immortalized cell lines, such as CHO cells, which have an overall increased genetic instability (15). Notably, the close similarity of CHO type-C ERVs to the MLV family, a retrovirus family known to cross the species barrier and to infect even primate cells (16), further indicates that CHO particles may have the potential to become pathogenic for humans, as seen for other retroviruses (17). Finally, CHO cell VLP were reported to contain viral genomic RNA sequences related to type-C retroviruses, as expected of viral particles (VP) (De Wit, C., Fautz, C., & Xu, Y. (2000). Real-time quantitative PCR for retrovirus-like particle quantification in CHO cell culture. Biologicals, 28(3), 137-148). However, the ERV sequences responsible for the release of the VLPs and VPs by CHO cells have remained uncharacterized. Hence strategies to avoid viral contaminations originating from CHO endogenous sources are highly desirable.

The most promising strategy to efficiently prevent hamster ERV transmission is to inactivate retroviruses using CRISPR-Cas9-mediated mutagenesis. The programmable CRISPR-Cas9 RNA-guided nuclease system has already been employed to introduce DNA double strand breaks (DSBs) into proviral sequences in human and porcine cells (18, 19). Imprecise DSB repair may lead to insertions and deletions within the viral sequences and inhibit viral activity. In a seminal paper, Yang et al. demonstrated that the CRISPR-Cas9 technology can be used to knock-out all 62 genomic porcine ERV sequences resulting in a more than 1000-fold reduction of ERV infectivity (19). Although successful, viral inactivation remains technically challenging, due to high cytotoxicity, frequent genomic rearrangements and low editing efficiency (19, 20). One explanation for the reduced editing efficiency of multi-loci sites compared to conventional editing of single genes might be the sheer number of ERV-like sequences that could serve as repair templates for precise, mutation-free repair, so antagonizing ERV mutagenesis and promoting chromosomal rearrangements. However, the incomplete characterization of type-C ERV sequences in CHO cells, as well as the absence of a clear link between the genomic type-C ERV sequences and viral particles, have hampered the establishment of a similar ERV inactivation strategy in CHO cells.

US Patent Publication 2019/0194694 A1, filed Dec. 23, 2016 discloses mammalian cells and mammalian cell lines that have a reduced load of remnants of past viral/retroviral infections and methods of producing and using the same. Engineered cells such as engineered CHO-K1 were disclosed therein. The engineering aimed at altering the genome by introducing alterations, preferably a high number of alterations, into ERVs in the genome of the cells to suppress or eliminate the release of VLPs and/or VPs. The complete consensus DNA sequence of functional Group 1 ERVs is shown in SEQ ID NO. 1 of US Patent Publication 2019/0194694 A1. The disclosure of US Patent Publication 2019/0194694 A1 is specifically incorporated herein by reference in its entirety.

There is a need in the art to engineer cells, such as CHO cells, so that they do not release or release substantially no potentially functional VPs. This is in particular of importance when the cells are designed to express any transgene product, in particular proteins with therapeutic activity. There is a need that the resulting engineered cells display little or none decrease in their transgene product production. There is a need in the art to provide such engineered cells, in particular for transgene product production. There is also a need to limit or abolish the presence of incompletely characterized retroviral nucleic acids in CHO culture supernatants. This and other needs are addressed herein.

### SUMMARY OF THE INVENTION

The budding type-C ERV sequences at the genome, transcriptome and viral particle level using CHO-K1 cells was characterized in-depth. In contrast to previous studies, transcribed type-C ERV group 1 sequences yielding full-length transcripts with open reading frames were identified, suggesting that this ERV group results in potentially functional retroviruses. Using CRISPR-Cas9 genome editing, the expressed group 1 type-C ERV sequences were mutated, and it could be shown that specific loss-of-function mutations within the gag gene of a single ERV suffices to decrease the release of functional viral RNA-loaded particles more than 250-fold. This indicated that a single ERV locus is responsible for most type-C viral particles released from CHO cells. Altogether, provided herein is a novel strategy to further improve the safety profile of CHO cells, paving the way to a complete eradication of endogenous viral contaminations in cultures of CHO cells producing biotherapeutics (also referred to herein as therapeutic products).

The invention is, in one embodiment, directed at an engineered cell, preferably of a mammalian cell line such as an engineered CHO cell, including an engineered CHO-K1, comprising: a genome of the cell comprising group 1 type-C ERV sequences including at least one full-length group 1 type-C ERV sequence(s) integrated into the genome, wherein the genome comprises one or more, but not more than twenty, including 19, 18, 17, 16, 15, 14, 13, 12, 11, 10, 9, 8, 7, 6, 5, 4, 3, 2 or 1 alteration(s) within one or more gag sequences of the group 1 type-C ERV sequences resulting in one or more altered group 1 type-C ERV sequences, wherein at least one of the alterations is within a gag gene of the at least one full-length group 1 type-C ERV sequence resulting in at least one altered full-length group 1 type-C ERV sequence.

The genome may comprise more than 100, more than 120, 140, 160, 180, 200, 220, 240, 260, 280 or 300 group 1 type-C ERV sequences, including at least one full-length group 1 type-C ERV sequence(s) integrated into the genome.

The at least one full-length group 1 type-C ERV sequence(s) integrated into the genome may correspond to SEQ ID 3 or sequences having more than 90%, 95%, 96%, 97%, 98% or 99% sequence identity therewith.

Of the more than 100, more than 120, 140, 160, 180, 200, 220, 240, 260, 280 or 300 group 1 type-C ERV sequences, more than 10, 20, 30, 40, 50, 60, 70 80, 90, 100 may be full-length group 1 type-C ERV sequence(s).

At least one of the at least one alteration within a gag gene of the at least one full-length group 1 type-C ERV sequence(s) may be a loss-of-function mutation.

The alteration(s) in the at least one full-length group 1 type-C ERV sequence(s) may block(s) translation initiation or may introduce a frameshift in the gag gene downstream of a PPYP motif.

The alteration(s) may be within the gag gene of not more than one of the full-length group 1 type-C ERV sequence(s), preferably within SEQ ID No. 3 more preferably within the Myr and/or PPYP Gag budding motifs or a sequence up to 5, 10, 15, 20, 25, 30, 35, 40, 45 or 50 nucleotides, including consecutive nucleotides, 5′ and/or 3′ of the Myr and/or PPYP Gag budding motifs.

The alteration(s) may comprise(s) a deletion of equal to or more than 1, 2, 3, 4, 5, 6, 7, 8, 9, 10 nucleotide(s), equal to or more than 1%, 2%, 3%, 4%, 5%, 10%, 20%, 30%, 40%, 50%, 60%, 70%, 80%, 90% or 100% consecutive nucleotides of SEQ ID NO: 3 or a sequence having more than 95%, 96%, 97%, 98%, 99% sequence identity therewith from the genome and optionally alterations in, including deletions of, nucleotide 1 to 30020, and 39348 to 59558 of Seq ID NO: 1.

Disclosed herein is also an engineered cell, preferably of a mammalian cell line such as an engineered CHO cell, including an engineered CHO-K1 cell, comprising:
a genome of the cell comprising:
a sequence comprising a gag gene, an env gene, a pol gene and long terminal repeats (LTR), and comprising at least one alteration in the gag gene, env gene, pol gene and/or the LTRs, wherein the sequence is selected from:
(i) SEQ ID NO: 3,
(ii) SEQ ID NO: 1,
(iii) a variant of (i) or (ii); or
(iv) a sequence having more than 95%, 96%, 97%, 98%, 99% sequence identity with (i) and/or (ii) outside the gag, env, pol gene and/or the LTRs,
said at least one alteration being selected from the group consisting of insertions, deletions, substitutions and combinations thereof.

The at least one alteration may be in the gag, env, pol gene and/or the LTRs is in not more than 100, 90, 80, 70, 60, 50, 40, 30, 20, 15, 10, 5, 4, 3, 2 nucleotides including consecutive nucleotides, or 1 nucleotide of the gag, env, pol gene and/or the LTRs.

Also disclosed herein is an engineered cell preferably of a mammalian cell line such as an engineered CHO cell, including an engineered CHO-K1 cell, wherein the genome comprises:
(i) not more than 10%, 20%, 30%, 40%, 50% consecutive nucleotides of SEQ ID NO: 3, or
(ii) a sequence having more than 90% sequence identity with (i).

The alteration(s) in the at least one full-length group 1 type-C ERV sequence(s) may be in the gag gene, that comprises a PPYP motif and wherein (i) sequences encoding the PPYP motif and/or a sequence up to 5, 10, 15, 20, 25, 30, 35, 40, 45 or 50 nucleotides, including consecutive nucleotides, 5′ and/or 3′ flanking the sequences in (i) may comprise the alteration(s).

The genome may comprise not more than 15, 14, 13, 12, 11, 10, 9, 8, 7, 6, 5, 4, 3, 2, 1 alteration(s) in the group 1 type-C ERV sequences.

The genome may comprise not more than 15, 14, 13, 12, 11, 10, 9, 8, 7, 6, 5, 4, 3, 2, 1 altered group 1 type-C ERV sequences.

The alteration(s) may be deletions, insertions, substitutions or combinations thereof, preferably alterations of the N-terminal Myr motif-encoding DNA sequence, such as one or several mutations that may inhibit the myristoylation of the GAG protein by removing or substituting the amino-terminal glycine residue, or a PPYP mutation that may inhibit the release of viral particles from the host cell, or one or several frameshift mutations that may infer with a translation of the gag mRNA into a full-length GAG protein.

The alteration(s) may be frameshift mutation(s).

The invention is, in a further embodiment, directed at an engineered cell, preferably of a mammalian cell line such as an engineered CHO cell, including an engineered CHO-K1, comprising:
a genome of the cell comprising group 1 type-C ERV sequences integrated into the genome, wherein at least one, including a singular, full-length group 1 type-C ERV sequence, such as SEQ ID NO: 3 or at least 10%, 20%, 30%, 40%, 50%, 60%, 70% 80% 90% or 100% consecutive nucleotides of SEQ ID NO: 3, and
optionally, 5′ and/or 3′ flanking regions of SEQ ID NO: 3 (i.e., sequences located 5′ and/or 3′ of SEQ ID NO: 3 in the genome), including 1-50, 30-100, 50-150, 100-200 or more than 200, 300, 400 or more than 500 consecutive nucleotides flanking SEQ ID NO: 3 are deleted from the genome.

The flanking regions may be SEQ ID NO: 4 and SEQ ID NO:5, respectively.

The genome of the cell may comprise: (i) at least 80%, 90%, 95%, 98%, 99% or 100% consecutive nucleotides of SEQ ID NO: 4 or sequences having at least 90%, 95%, 98% or 99% sequence identity therewith and, directly adjacent thereto, at least 80%, 90%, 95%, 98%, 99% or 100% consecutive nucleotides of SEQ ID NO: 5 or sequences having at least 90%, 95%, 98% or 99% sequence identity therewith. Preferably, SEQ ID NO: 4 is 5′ of SEQ ID NO: 5 in the resulting sequence.

The alteration(s) may be insertions of at least 5, 10, 15, 20, 25, 30, 50 or 100 nucleotides, deletions of at least 5, 10, 15, 20, 25, 30, 50 or 100 nucleotides, including consecutive nucleotides, or combinations thereof or combinations of insertions, substitution and/or deletions resulting together in an addition and/or removal of at least 5, 10, 15, 20, 25, 30, 50 or 100 nucleotides.

The ERV elements may be from gamma or beta retroviral ERVs, including Intracisternal Leukemia Virus, Koala epidemic viral (KoRV), Mouse Mammary Tumor Viral (MMTV), Mouse Leukemia Viral (MLV) ERVs, Feline Leukemia Virus, Gibbon Ape Leukemia Virus, Porcine Type-C Endogenous Retrovirus and/or Intracisternal Leukemia Virus.

The engineered cell may release a number of viral particles (VPs), viral like particles (VLPs) and/or retroviral (like) particles (RV(L)Ps) per unit of time, the number being reduced, preferably more than 2-fold, more preferably more than 10-fold, even more preferably more than 50-fold, more than 100-fold, more than 150-fold, more than 200-fold or more than 250-fold relative to the VPs, VLPs and/or RV(L)Ps per unit of time released by its non-engineered counterpart.

The engineered cell may release no or substantially no VPs and/or VLPs, in particular substantially no RVPs and/or RVLPs.

The engineered cell may further comprise a transgene, preferably integrated into the genome.

The transgene may be a marker gene encoding a marker protein such as GFP (green fluorescent protein), a biotherapeutic and/or a non-coding RNA.

The invention is, in a further embodiment, directed at an engineered cell, preferably of a mammalian cell line such as an engineered CHO cell, including an engineered CHO-K1, comprising:
a genome of the cell comprising SEQ ID NO: 3 or a variant thereof, and further comprising a sequence encoding a siRNA, wherein a target sequence of the siRNA is located within SEQ ID NO:3, preferably within a sequence of SEQ ID NO:3 or a variant thereof, more preferably within sequence of SEQ ID NO: 3 encoding the Gag precursor protein or a variant thereof.

The invention is, in a further embodiment, directed at a method for producing a transgene product comprising:
providing the engineered cell(s) of any one of the preceding claims,
introducing at least one transgene encoding the transgene product, such as a biotherapeutic, into the engineered cell, and
expressing the at least one transgene in the cell, wherein said engineered cell releases no or substantially no VLPs.

Disclosed is also a detection kit and its use comprising:
(i) at least one primer against SEQ ID NO: 3, and/or
(ii) at least one primer against SEQ ID NO: 4 or 5, and
instructions how to use the primers of (i) and/or (ii) to detect the presence or absence of SEQ ID NO: 1, of SEQ ID NO: 3 from a genome of a CHO cell or a mutation within SEQ ID NO: 3 of the genome of the CHO cell.

### BRIEF DESCRIPTION OF THE FIGURES

FIG. 1. Phylogenetic analysis of full-length type-C ERV DNA sequences within the CHO-K1 genome. Sequences alignments were realized with MAFFT aligner version 7 (64), with standard parameters, a scoring matrix of 200PAM/k=2, a gap open penalty of 2.55 and an offer value of 0.123. Alignments were realized for the complete gag-pol-env sequence (A) or in the gag (B), pol (C) and env (D) sequences separately of 112 full-length type-C ERV. From these alignments, phylogenetic trees were made using GENEIOUS Tree Builder version 11.1.5 and using the genetic distance model HKY and the method of UPGMA based on sequence similarity. The scale under the tree show the substitution rate per nucleotide. Group 1 with its three subclusters and group 2 are indicated by brackets.

FIG. 2. Characterization of expressed type-C ERV sequences in wild-type CHO cells. Mapping of Illumina sequencing reads of total cellular RNA (A), or of viral particle RNA (B) obtained from CHO-K1 cells on group 1 and group 2 type-C ERV sequences. Reads were mapped to a consensus sequence for group 1 and on two distinct loci (locus A and locus B) for group 2 ERV. The maximal number of mapped reads is indicated on the left axis of each panel, and the Reads Per Kilobase Million (RPKM) are stated to the right. (C) Representative metaphase spread of CHO-K1 chromosomes FISH analysis using fluorescent probes specifically targeting group 1 type-C ERV. Chromosomal DNA is represented and the FISH signals of integrated retroviral sequences are shown as lighter dots (D) Three representative interphase CHO-K1 cells are shown, with mRNAs showing in the central region and group 1 type-C ERV RNA at the periphery. The bright light dot represents the nascent group 1 mRNA at the transcription site. Signs present on the schematic representation of group 2 ERV on locus A and B (A) show the mutation type occurring in these ERV sequences, 2 frameshift mutations N terminally and 3 stop codon mutations in the depiction (left) of C-type group 2 ERV-locus A and 2 deletions in the depiction (left) of C-type group 2 ERV-locus B. with the deletion size indicated as the number of bases.

FIG. 3. CRISPR-Cas9 target sites for ERV mutagenesis. The orientation and position of the eight sgRNA sequences designed to target the Myristoylation (Myr) and PPYP motifs of the gag group 1 type-C ERVs are illustrated by grey arrows. The CRISPR-Cas9 DSB sites are shown by open triangles for sgRNAs targeting the forward strand (Myr2, PPYP5, PPYP6, PPYP7) and by filled triangles for sgRNA targeting the reverse strand (Myr4, Myr8, PPYP13, PPYP20). The Protospacer adjacent motif (PAM) sites are marked by bold letters.

FIG. 4. Assessment of sequence diversity in the Myr and PPYP motif flanking regions and analysis of CRISPR-derived mutations by deep DNA sequencing. Targeted amplification of approximately 300 bp surrounding the Myr and PPYP CRISPR target sites was performed using type-C ERV specific primers and amplicons were analyzed by Illumina deep sequencing. Clustering analysis was based on 97% similarity of wild-type CHO-K1 deep sequencing reads from the Myr (A) and PPYP (B) flanking sequences. Clusters are indicated by brackets according to the phylogenetic groups identified in FIG. 1. Clusters containing the Myr2 sgRNA and PPYP6 sgRNA recognition sites and an adjacent PAM sequence are shown in bold, and the most abundant cluster per target site is Cluster 8 for the Myr (A)/(E) and cluster 2425 for PPYP (B)/(F). Values to the right represent the frequency of reads obtained for each subcluster relative to the total number of reads. (C) Number of distinct mutations and their corresponding read frequencies in seven clones (C02, D12, G09, A02, E10, K03, K14) isolated from Myr2 or PPYP6 sgRNA-treated polyclonal populations. All clones display mutations in the expressed group 1 type-C ERV locus. Grey shaded boxes represent mutations occurring at a higher than average read frequency (>0.4%, left-hand side axis) and the predicted number of ERV loci containing an identical mutation is indicated as dashed lines. The estimated total number of mutated ERV loci of each clone is indicated by the right-hand side axis. (D) Frequency of Myr2 or PPYP6 sgRNA-induced repair junctions compatible with C-NHEJ, alt-EJ or HR-mediated gene conversion DSB repair mechanisms. Repair junctions incompatible with these three main DSB repair mechanisms are grouped as Unknown. A total of 74 DNA repair junctions (n_{Myc}=47, n_{PPYP}=0.27) obtained from both Sanger mRNA and Illumina deep DNA sequencing were analyzed. (E and F) Frequency of the wild-type CHO clusters representing best the mutation-flanking sequence of 30 Myr2- and 12 PPYP6-derived mutation deep sequencing reads. Clusters containing the Myr2 or PPYP6 sgRNA recognition sites including an adjacent PAM site are shown in bold letters (on-targets), while clusters with sgRNA mismatches are shown in normal letters (off-targets). Off-target cluster possesses mismatches at position 13 or 15 in the sgRNA recognition site.

FIG. 5. Viral particle RNA sequencing of CHO clones mutated in the expressed group 1 type-C ERV sequence. Mapping of viral RNA particle deep sequencing reads from a Myr2 sgRNA clone (D12, left panels) and a PPYP6 sgRNA clone (E10, right panels) on group 1 consensus sequence and group 2 locus A and locus B. were performed as shown for the wild-type CHO viral particles (FIG. 2B). D12 and E10 mutants both contain Gag loss-of-function mutations in the functionally relevant group 1 type-C ERV locus. The number of reads mapping to each panel is indicated on the left axis and the Reads Per Kilobase Million (RPKM) are stated to the right.

FIG. 6. Assessment of cell growth, cell size and therapeutic IgG Immunoglobulin production In ERV-mutated CHO cells. Viable cell density (A) and cell size (B) was measured in wild-type CHO cells (WT), empty sgRNA vector-treated cells (Empty), bulk-sorted polyclonal CRISPR-treated cells (Poly) as well as in clones containing mutations in the expressed ERV locus (C02, D12, G09, A02, E10, K03 and K14) or not (B01, B03) after five days of culture. The same samples were stably transfected to express an IgG Immunoglobulin antibody and assessed for cell density (C), cell viability (D) and IgG production (E) during ten-days fed-batch cultures. Statistical significance relative to the empty vector control was calculated using the two-tailed unpaired Student's t-test with Benjamini and Hochberg false discovery rate correction (n≥2, error bars represent s.e.m, *P<0.05,**P<0.01).

FIG. 7. Assessment of gag-specific sgRNA-mediated CRISPR-Cas9 cleavage by flow cytometry. Analysis of the of dsRed positive (dsRed+) cell frequency (A), the dsRed fluorescence intensity (B) and the frequency of high granularity cells (C and D) of CHO cells transfected with CRISPR-Cas9, Myr or PPYP motif-specific sgRNAs (Myr2, Myr4, Myr8, PPYP5, PPYP6, PPYP7, PPYP13, PPYP20 sgRNAs) or a non-targeting empty vector control and dsRed transfection control expression plasmids. Panel C shows size (FSC) vs granularity (SSC) flow cytometry density plots of the empty vector-, Myr2 sgRNA- and PPYP6-treated cells. The larger gate selects for intact non-debris cells while the smaller gate marks the CHO cell subpopulation with an elevated granularity level, as quantified in panel D. Statistical significance relative to the empty vector control was calculated using the two-tailed unpaired Student's t-test with Benjamini and Hochberg false discovery rate correction (n=3, error bars represent s.e.m, * P<0.05, ** P<0.01).

FIG. 8. Estimation of gag-specific sgRNA-mediated CRISPR-Cas9 cleavage efficiency by targeted mRNA sequencing of polyclonal CHO populations. Indel mutation analysis of polyclonal PCR products obtained from reverse-transcribed cellular mRNA of bulk-sorted CRISPR-treated polyclonal populations using the indicated group 1 type-C specific primers. The mutation frequency was estimated by decomposition of the Sanger chromatogram (28). The predicted mutation frequency relative to the untreated wild-type control sample is shown on the right of the chromatograms. The DSB site for each sgRNA is shown with a black line and the decomposition window, downstream of the DSB site relative to the sequencing direction indicated by an arrow, is shaded in grey. The Myr motif shown corresponds to nucleotides 10-71 of SEQ ID NO: 86. The PPYP motif shown corresponds to nucleotides 21-98 of SEQ ID NO: 76.

FIG. 9. Wild-type CHO consensus sequences of Myr and PPYP diversity clusters. Cluster sequences of Myr (A) and PPYP (B) flanking regions of deep-sequenced wild-type CHO cells. Shades correspond to the phylogenetic groups depicted in FIGS. 4A and 4B. Myr and PPYP clusters containing a sgRNA recognition site (black outlined arrow) with an adjacent PAM sequence are written in bold letters. Myr and PPYP motifs are indicated with very light grey and dark grey outlined boxes, respectively. The higher sequence complexity of the PPYP flanking region relative to the Myr flanking region is illustrated by missing sequences or lines depicting deletions or insertions and single nucleotide variants, respectively.

FIG. 10. Characterization of ERV locus-specific mutations and their frequencies within clonal populations. Analysis of Illumina raw reads of mutations detected at normal (0.2-0.4%) or high (>0.4%) read frequencies in different clones. Pie charts represent number and frequency of identified groups with identical CRISPR-derived mutation but distinct mutation flanking sequences (e.g. in D12_1_1 and G09_1_1). The 51% marked part of the pie provides 4 about equal sections indicating the number of predicted ERV loci that could not be distinguished based on their flanking sequences.

FIG. 11. Characterization of Myr2 sgRNA- and PPYP6 sgRNA-mediated mutations and repair junctions. 47 Myr2- and 27 PPYP6-derived repair junctions were analyzed for sgRNA specific mutation signatures, including the elicited mutation type (Deletion, Insertion, Indel) (A), the mutation effect on Gag and ERV function (Outside ERV coding region, Translational inhibition, Frameshift mutation, In-frame mutation) (B), the mutation size distribution (C) and MMEJ and SD-MMEJ alt-EJ repair pathway activities. Indel mutations are defined in this figure and throughout this specification as deletions coupled to insertions. Repair junctions compatible with both MMEJ and SD-MMEJ repair mechanisms are classified as “MMEJ+SD-MMEJ”. Repair junctions were obtained from both Sanger mRNA and Illumina DNA deep sequencing.

FIG. 12. Identification of a unique functionally active group 1 type-C ERV locus. (A) Schematic representation of a 15 kb PacBio read obtained following whole-genome sequencing of the E10 (PPYP6 sgRNA) clone. The read contains full-length gag, pol, env and full-length 3′ LTR sequences as well as the E10-specific CRISPR-mutation in the gag gene and extends into the CHO genome. (B) Alignment of the PacBio CHO genome-specific sequence against the publicly available NCBI CHO genome. The NCBI scaffold identifier is shown on top. The predicted group 1 type-C ERV integration site is indicated. The genomic region surrounding the ERV integration site contains two protein-coding genes (Cidec, Jagn1) as well as three pseudogenes (Rps15, Rpl18a, Rpl34; shown with light grey backgrounds), as annotated by the NCBI. Cidec (cell death inducing DFFA like effector c) encodes for a lipid droplet protein involved in lipid metabolism (65), Jagn1 (jagunal homolog 1) encodes for an endoplasmatic reticulum protein involved in the early secretory pathway (66) and Rps15, Rpl18a, Rpl34 encode for ribosomal proteins. The predicted mRNA expression levels for each gene are estimated by RNA sequencing data and expressed as Reads Per Kilobase Million (RPKM). (C) Sanger sequencing results of the Myr2 and PPYP6 sgRNA flanking regions. Sanger sequencing was performed on PCR amplicons obtained from total cellular mRNA using group 1 specific primers (“mRNA” in the figure) or genomic DNA using primers specific to the expressed group 1 type-C ERV (“DNA” in the figure). Clones C02, D12, G09, A02, E10. K03, K14 contain mutations in the functionally active group 1 type-C ERV locus, but clones (B01 and B03) as well as the empty vector controls do not. The predicted Myr2 and PPYP6 DSB sites are marked with a dotted line.

FIG. 13. Assessment of viral RNA amounts released in VP by ERV-mutated CHO cells. The retroviral RNA genomes were isolated from viral particles present within the supernatants of five-day cultures of untreated cells (UT), empty sgRNA vector-treated cells (Empty), bulk-sorted polyclonal CRISPR-treated cells (Poly), as well as clones containing mutations in the expressed type-C group 1 ERV locus (C02, D12, G09, A02, E10, K03 and K14) or without a detected ERV mutation (B01, B03). (A) The RNA was processed for Illumina sequencing and the obtained reads were mapped onto the group 1 type-C ERV locus of SEQ ID NO: 3. The ERV reads were mapped to the sequences of the expressed group 1 ERV locus of SEQ ID NO: 3 (grey bars) and to the 45S ribosomal RNA (black bars) sequences of CHO cells used as a control. The y-axis represents the number of reads per kilobases for each sequencing reaction. (B) Quantitative PCR (q-PCR) analysis of the reverse-transcribed total RNA isolated from VP released in cell culture supernatants. Reverse transcription and q-PCR analysis was performed in triplicates from samples obtained from 3 independent CHO cell cultures. The genomic retroviral sequences were quantified using group 1 ERV LTR-specific primers. Data were normalized to the number of analyzed cells and are represented as the average and standard deviation of the fold change relative to those of UT cells.

### DETAILED DESCRIPTION OF VARIOUS AND PREFERRED EMBODIMENTS

A cell, preferably a mammalian cell/eukaryotic cell, that according to the present invention includes an engineered cell, is capable of being maintained under cell culture conditions. Standard cell culture conditions are from 30 to 40° C., preferably at or at about 37′C, for instance in fully synthetic culture medium as used in the production of recombinant proteins. Non-limiting examples of this type of cell are non-primate eukaryotic cells such as Chinese hamster ovary (CHOs) cells including the CHO-K1 (ATCC CCL 61), DG44 and CHO-S cells and SURE CHO-M cells (derivative of CHO-K1), and baby hamster kidney cells (BHK, ATCC CCL 10). Primate eukaryotic host cells include, e.g., human cervical carcinoma cells (HELA, ATCC CCL 2) and 293 [ATCC CRL 1573] as well as 3T3 [ATCC CCL 163] and monkey kidney CV1 line [ATCC CCL 70], also transformed with SV40 (COS-7, ATCC CRL-1587). The term engineered signifies that the genome of the cell has been altered, e.g., by insertion(s), deletion(s) and/or substitution(s). As the person skilled in the art will readily understand the cells that are being engineered, even prior to engineering as described herein, are non-naturally occurring cells. The above-mentioned cells, in particular, the various CHO cells, are commonly used in biotechnological applications, such as for the production of therapeutic proteins. As the person skilled in the art will also readily understand, other cells than the ones mentioned above might be engineered as long as they are used or can be used in biotechnological applications, in particular for the expression of, e.g., therapeutic proteins.

Endogenous retroviruses (ERVs) are sequences that derived from ancient retroviral infections of germ cells and integrated in mammal and other vertebrate cells millions of years ago. These ERVs are inherited according to Mendelian laws. The size of a complete endogenous retrovirus is between 6-12 kb on average and it contains gag, pol and env genes that always occur in the same order. Coding sequences are flanked by two LTRs (Long Terminal Repeat sequences). Most ERVs are defective, as they are carrying a multitude of inactivating mutations. In addition, they can be inactivated (i.e. not transcribed) by epigenetic silencing effects. However, some ERVs still have open reading frames in their genome and/or they may be transcriptionally active. The ERVs of mammals bear strong similarities and may originate from the genus of gammaretroviruses and betaretroviruses, including Intracisternal Leukemia Virus, Feline leukemia virus (FeLV), Mouse Leukemia Virus (MLV), Koala epidemic virus (KoRV), Mouse Mammary Tumor Virus (MMTV). ERVs are maintained in the genomes and may have certain advantages for the cells into whose genome they are integrated, including providing a source of genetic diversity and protection against other viral pathogens. However, they can become infectious and carry risks in in the context of transgene, i.e. protein, expression described elsewhere herein, in particular, as a result of ERV awakening due to cancer, cellular stress and/or epigenetic modifications.

The three major proteins encoded within the retroviral genome are Gag, Pol, and Env. Gag (Group Antigens) encoded by the gag gene is a polyprotein, which is processed to matrix and other core proteins, including the nucleoprotein core particle, that determines the retroviral core. Pol is the reverse transcriptase, encoded by the pol gene and has RNase H and integrase function. Its activity results in the double-stranded DNA pre-integrated form of the virus and, via the integrase function, for the integration into the host genome, and also via the RNase function, the reverse transcription after integration into the genome of the host. Env is the envelope protein, encoded by the env gene, and resides in the lipid layer of the virus determining the viral tropism.

US Patent Publication 2019/0194694 A1. filed Dec. 23, 2016 demonstrated the three classes of gammaretroviruses that might be integrated into the genome of the cells to form gammaretrovirus-related ERVs. 159 IAP (Intracisternal A-type particles) sequences and 144 type C murine ERV-like sequences were previously reported, as well as 6 sequences related to GALV (Gibbon Ape Leukemia Virus).

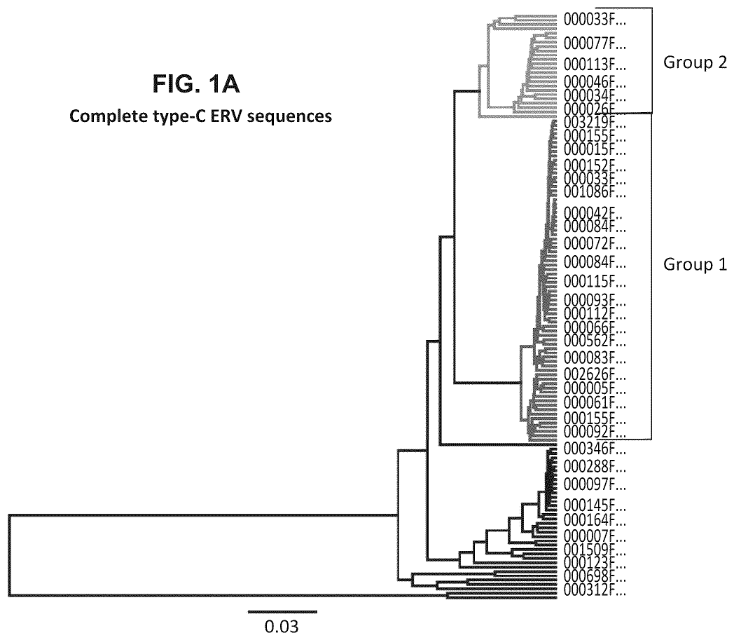

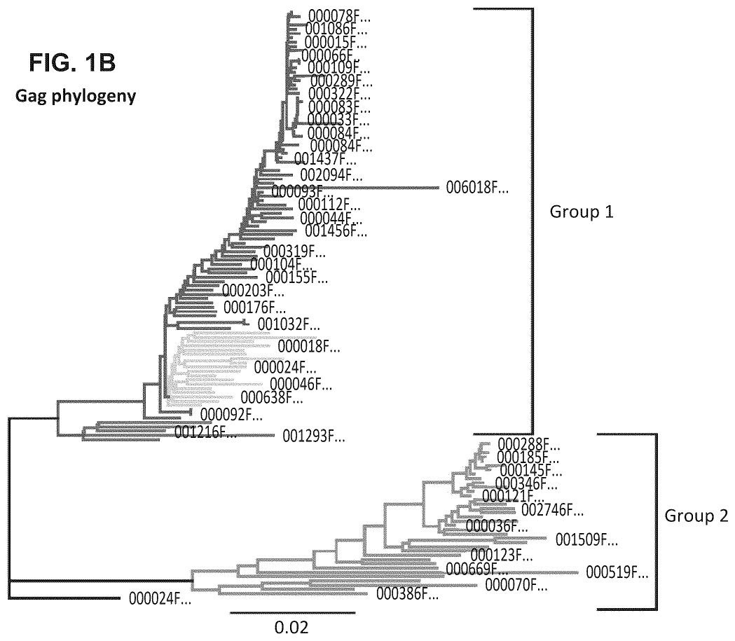

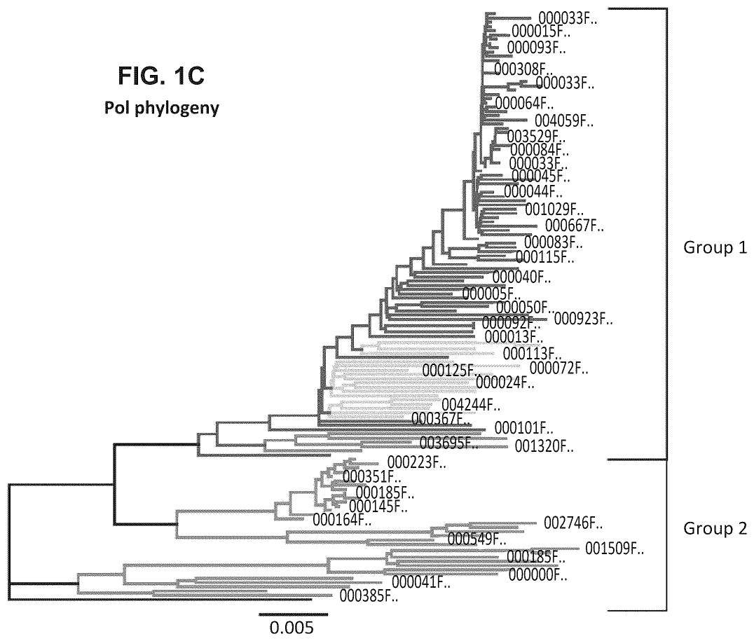

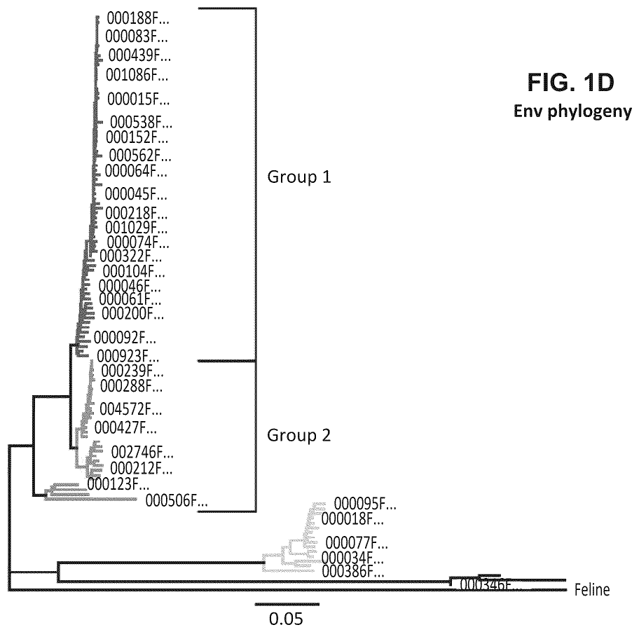

A neighbor-joining consensus tree based on 121 GAG sequences of the gamma retrovirus-like ERVs from a CHO genome was also discussed in US Patent Publication 2019/0194694 A1, filed Dec. 23, 2016. Both group 1 and 2 ERVs were shown to contain transcriptionally active ERVs. One sequence in the group 2 ERVs was found to be active, but contained stop codons. In contrast multiple sequences in group 1 were found to be active and not to contain a stop codon in the coding sequence. A Gag and Pol cDNA analysis was consistent with the existence of expressed ERVs encoded by full-length ERV sequences. Based on those sequences, a consensus sequence of group 1 viruses was determined as gcccccgcca tatccgccac tgccgccccc accagaggca gaagcgg [SEQ ID NO: 6]. Compare FIGS. 1A, B, C and D.

Full length ERV sequences, in particular full-length group 1 type-C ERV sequences, are sequences that are integrated into the genome of a cell and, prior to introducing an alteration, can be expressed, that is, transcribed into functional transcripts with intact open reading frames of the gag, pol and env genes. Thus, a full-length ERV sequence, in particular full-length group 1 type-C ERV sequence, will encode, at a minimum, a Gag-precursor protein, a Pol encoded reverse transcriptase, and an Env protein. In preferred embodiments a full-length ERV sequence also includes one or both long terminal repeats (LTRs) or portions thereof, such as 10, 20, 30, 40, 50, 60, 70, 80% consecutive nucleotides thereof. In an even more preferred embodiment, the full-length and expressed ERV sequence corresponds to SEQ ID NO: 3 or a sequence having more than 90%, 95%, 98% or 99% sequence identity therewith.

Some of the full-length group 1 type-C ERV sequences might lead to the formation and release of viral particles (VPs) that might comprise the full-length viral genomic RNA packaged into the viral particles. In the context of the present application VPs refer to viral particles that contain at least a part of a viral genome. In some instances, the VPs may comprise the full-length viral genomic RNA and thus may be functional VPs. VLPs as used in the context of the present invention are particles that appear to be VPs, but lack any part of the viral genome.

A loss of function mutation interferes with proper protein synthesis. ergo no functional protein is synthesized if such a mutation occurs. In the case of a loss of function mutation in, e.g., a gag gene, the Gag-precursor protein or one of its cleavage products is compromised so that ERV budding does not take place.

The engineered cell according to the present invention, may comprise a genome that, in most parts, is identical to the genome of the cell it is derived from, such as a CHO-K1 cell. However, at least one and not more than 20, including 19, 18, 17, 16, 14, 13, 12, 11, 10, 9, 8, 7, 6, 5, 4, 3, 2, 1 ERV sequences, including group 1 type-C ERV sequences, which are part of these genomes will contain alterations as described herein.

The gag gene gives rise to a Gag precursor protein, which is expressed from the unspliced viral mRNA. The Gag precursor protein is cleaved by the virally encoded protease (a product of the pol gene) during the process of viral maturation into generally four smaller proteins designated MA (matrix), CA (capsid), NC (nucleocapsid), and a further protein domain (e.g. pp12 in murine leukemia virus (MLV) or p6 in HIV). A gag sequence as referenced herein may or may not give rise to a Gag precursor protein.

The gag gene encodes an N-terminal Myr motif, located downstream of the ATG translation initiation codon. Alterations in the Myr motif are part of the present invention. Such alterations generally interfere with Gag myristoylation and, e.g., block translation or create a loss-of-function mutated gag transcript. As a result, the proper viral particle assembly at the plasma membrane and/or retroviral particle release may, in certain embodiments of the invention, be blocked. The Myr motif of SEQ ID NO: 3 is encoded by sequences located at 1334-1336 (atg ggg caa). The Myr motif is also referred to herein as the Myr budding motif.

The PPxY motif of the gag gene also contributes to retrovirus budding. Alterations in the PPXY motif are also part of the present invention. Such alterations may strongly inhibit viral particle release. The PPxY motif may overtap with a PPYP motif (or the PPYP budding motif) that is conserved in group 1 and group 2 CHO ERVs, which is termed PPYP hereafter to refer to this CHO-specific PPxY-related budding motif. The PPYP is encoded by the sequences located at 1851-1868 (ccc ccg cca tat ccg cca) of SEQ ID NO: 3.

The MA polypeptide is derived from the N-terminal, myristoylated end of the precursor protein. Most MA molecules remain attached to the inner surface of the virion lipid bilayer, stabilizing the particle.

The CA protein forms the conical core of viral particles.

The NC region of Gag is responsible for specifically recognizing the so-called packaging signal of the retrovirus. The packaging signal comprises four stem loop structures located near the 5′ end of the viral RNA, and is sufficient to mediate the incorporation of a heterologous RNA into virions. NC binds to the packaging signal through interactions mediated by two zinc-finger motifs.

Another protein domain mediates interactions between precursor protein Gag and the accessory protein Vpr, leads to the incorporation of Vpr into assembling virions. The p6 region in HIV also contains a so-called late domain which is required for the efficient release of budding virions from an infected cell. (Hope & Trono, 2000).

The viral protease (Pro), integrase (IN), RNase H, and reverse transcriptase (RT) are expressed within the context of a Gag-Pol fusion protein. The Gag-Pol precursor is generally generated by a ribosomal frame shifting event, which is triggered by a specific cis-acting RNA motif (a heptanucleotide sequence followed by a short stem loop in the distal region of the Gag RNA). When ribosomes encounter this motif, they shift approximately 5% of the time to the pol reading frame without interrupting translation. The frequency of ribosomal frameshifting explains why the Gag and the Gag-Pol precursor are produced at a ratio of approximately 20:1.

During viral maturation, the virally encoded protease cleaves the Pol polypeptide away from Gag and further digests it to separate the protease, RT, RNase H, and integrase activities. These cleavages do not all occur efficiently, for example, roughly 50% of the RT protein remains linked to RNase H as a single polypeptide (p65) (Hope & Trono, 2000).

The pol gene encodes the reverse transcriptase. During the process of reverse transcription, the polymerase makes a double-stranded DNA copy of the dimer of single-stranded genomic RNA present in the virion. RNase H removes the original RNA template from the first DNA strand, allowing synthesis of the complementary strand of DNA. The predominant functional species of the polymerase is a heterodimer. All of the pol gene products can be found within the capsid of released virions.

The IN protein mediates the insertion of the proviral DNA into the genomic DNA of an infected cell. This process is mediated by three distinct functions of IN.

The Env protein is expressed from singly spliced mRNA. First synthesized in the endoplasmic reticulum, Env migrates through the Golgi complex where it undergoes glycosylation. Env glycosylation is generally required for infectivity. A cellular protease cleaves the protein into a transmembrane domain and a surface domain. (Hope & Trono, 2000).

The viral genomic RNA expressed from some ERVs of a genome can be released from the cells in the form of VPs. Other expressed ERVs may cause the formation of RVLPs but not of VPs, and thus may not be released in the form of a viral genomic RNA.

However, generally the ones that are released have a higher potential to become infectious.

Thus, it is generally advantageous to have cells engineered, as described herein, that can express and release no or substantially no VPs, preferably also no VLPs, preferably under both standard or stressful culturing conditions. Substantially no VPs/VLPs are released if a cell culture comprising the so engineered cell releases less than 50%, less than 40%, less than 30%, less than 20%, less than 10%, preferably less than 5% of VPs/VLPs than their counterpart that has not been subjected to the VPs/VLP release reducing procedures described herein. Such a counterpart would. e.g., be a commercially available CHO-K1 cell. No or substantially no expression means that less than 50%, less than 40%, less than 30%, less than 20%, less than 10%, preferably less than 5%, unmutated Gag mRNA sequence can be detected by PCR and sequencing analysis. No release means that no, or substantially no detectable viral sequence release occurs as assessed via a PureLink Viral RNA/DNA extraction Kit® INVITROGEN and a cDNA PCR assay, or as obtained from QIAGEN, QuantiTect Rev. Transcription Kit®.

Alteration(s) to a sequence or gene include addition(s)/insertion(s), deletion(s) and/or substitution(s) that do not occur in the cells, in particular in one or more, including one or more specific, ERVs of the cell, prior to engineering as described herein. In certain embodiments the alteration(s) might encompass the excision of at least one, in certain embodiments just one, that is a singular, entire ERV including optionally flanking regions 5′ and/or 3′ of the ERV. The alteration may include, for example, at least one alteration in the gag, env, pol gene and/or the LTRs. In certain embodiments the alteration comprises not more than 100, 90, 80, 70, 60, 50, 40, 30, 20, 15, 10, 5, 4, 3, 2 nucleotides including consecutive nucleotides, or 1 nucleotide of the gag, env, pol gene and/or the LTRs, of in particular one or more ERV sequences such as a full length ERV sequences and/or one or more specific sequences disclosed herein.

A heterologous nucleic acid sequence is a nucleic acid sequence that does not occur in the cells prior to engineering according to the present invention, while related types of nucleic acid sequences may very well exist in the cell. A transgene as used in the context of the present invention is such a heterologous nucleic acid sequence, in particular a deoxyribonucleotide (DNA) sequence coding for a given mature protein (also referred to herein as a DNA encoding a protein), for a precursor protein or for a functional RNA that does not encode a protein (non-coding RNA). A transgene is isolated and introduced into a cell to produce the transgene product. Some preferred transgenes according to the present invention encode marker proteins such as GFP (green fluorescent protein). Those can be used to detect successful integration into, ergo alteration/inactivation of, ERV elements. Other transgenes are those that encode, e.g., proteins that shall ultimately be produced by the cell in question such as immunoglobulins (Igs) and Fc-fusion proteins and other proteins, in particular proteins with therapeutic activity (“biotherapeutics”).

As used herein, “genome editing” refers to the modification (“editing”) of genomic sequences and may comprise a deletion of at least one nucleotide, an addition/insertion of at least one nucleotide, or a substitution of at least one nucleotide. The genomic sequence edited is referred to herein as target nucleic acid sequence. Targeted insertions are insertions that occur at a specific predetermined target site. Genome editing tools introduce double or single stranded breaks into the genome, e.g., via nucleases or nickases, and rely at least in part on the cellular recombination mechanisms (see discussion below) to repair these breaks. These tools also contain generally sequence specific DNA binding modules.

ZFNs (Zinc-Finger Nucleases) and TALENs (transcription activator-like effector nucleases) enable a broad range of genetic modifications by inducing DNA double-strand breaks (DSBs) that stimulate error-prone non-homologous end joining (NHEJ) or homology-directed repair (HDR) at specific genomic locations.

The sequence specificity of CRISPR (clustered, regularly interspaced, short palindromic repeats) systems is determined by small RNAs. CRISPR loci are composed of a series of repeats separated by‘spacer’ sequences that match the genomes of bacteriophages and other mobile genetic elements. The repeat-spacer array is transcribed as a long precursor and processed within repeat sequences to generate small crRNA that specify the target sequences (also known as protospacers) cleaved by CRISPR systems. For cleavage, the presence of a sequence motif immediately downstream of the target region is often required, known as the protospacer-adjacent motif (PAM). CRISPR-associated (cas) genes usually flank the repeat-spacer array and encode the enzymatic machinery responsible for crRNA (CRISPR RNA) biogenesis and targeting. Cas9 is a dsDNA endonuclease that uses a crRNA guide to specify the site of cleavage. Loading of the crRNA guide onto Cas9 occurs during the processing of the crRNA precursor and requires a small RNA antisense to the precursor, the tracrRNA, and RNAse Ill. In contrast to genome editing with ZFNs or TALENs, changing Cas9 target specificity does not require protein engineering but only the design of the short crRNA guide, also termed sgRNA.

To date, three different variants of the Cas9 nuclease have been adopted in genome-editing protocols. The first is wild-type Cas9, which can site-specifically cleave double-stranded DNA, resulting in the activation of the doublestrand break (DSB) repair machinery. DSBs can be repaired by the cellular Non-Homologous End Joining (NHEJ) pathway, resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made.

The Cas9 system was further engineered towards increased precision by developing a mutant form, known as Cas9D10A, with only nickase activity. This means it cleaves only one DNA strand, and does not activate NHEJ. Instead, when provided with a homologous repair template, DNA repairs are conducted via the high-fidelity HDR pathway only, resulting in reduced indel mutations. Cas9D10A is therefore in many applications more appealing in terms of target specificity when loci are targeted by paired Cas9 complexes designed to generate adjacent DNA nicks.

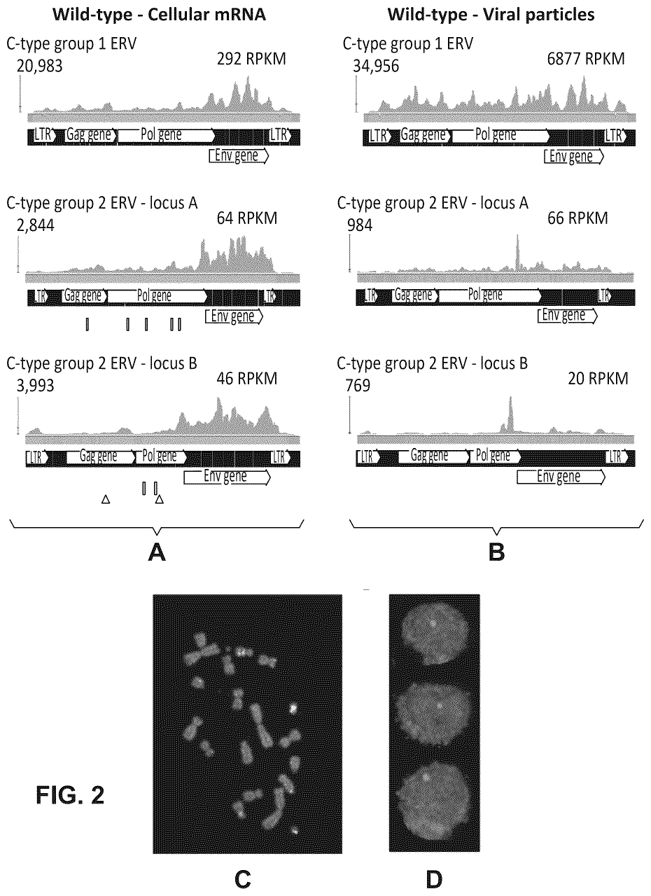

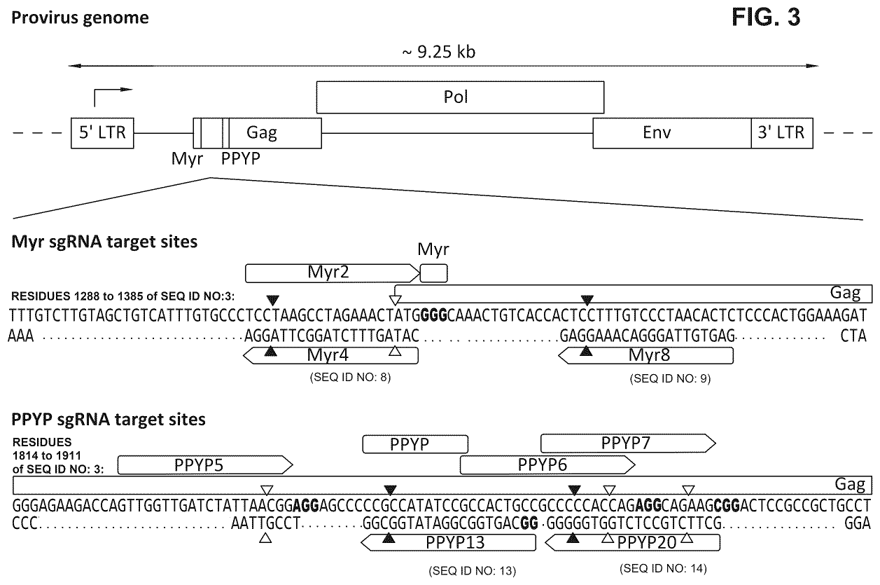

In the context of the present invention, a specific sequence or a consensus sequence of ERV elements are determined to specify the site of cleavage via, e.g., one of the systems above. Such a specific or consensus sequence is preferably between 5 and 50 base pairs long, preferably between 10 and 50 or between 15 and 25 or between 25 and 50 or 30 and 50. The consensus sequences may contain, e.g., 1, 2, 3, 4 or 5 mismatches (have more than 60%, 70%, 80%, 90% or 95% complementarity relative to each other), as long as cleave can still be performed. See, e.g, FIG. 3 and Table 1 that show specific target sites for Myr- and PPYP-specific sgRNAs in the CHO-K1 genome. The above systems are called non-naturally occurring systems or heterologous systems, which means that they are introduced to the cell rather than being a part of the cell prior to engineering according to the present invention. The specific DNA cleavage events lead, in certain embodiments, to transcriptional silencing of expressed ERVs.

A vector according to the present invention is a nucleic acid molecule capable of transporting another nucleic acid, such as a transgene that is to be expressed by this vector, to which it has been linked, generally into which it has been integrated. For example, a plasmid is a type of vector, a retrovirus or lentivirus is another type of vector. In a preferred embodiment of the invention, the vector is linearized prior to transfection. An expression vector comprises heterologous regulatory elements or is under the control of such regulatory elements that are designed to further the transcription and/or expression of a nucleic acid sequence, such as a transgene, carried by the expression vector. Regulatory elements comprise enhancers and/or promoters, but also a variety of other elements described herein.

Among non-viral vectors, transposons are particularly attractive because of their ability to integrate single copies of DNA sequences with high frequency at multiple loci within the host genome (integrating vector). Unlike viral vectors, some transposons were reported not to integrate preferentially close to cellular genes, and they are thus less likely to introduce deleterious mutations. Moreover, transposons are readily produced and handled, comprising generally of a transposon donor vector containing the cargo DNA flanked by inverted repeat sequences and of a transposase-expressing helper plasmid or mRNA. Several transposon systems were developed to mobilize DNA in a variety of cell lines without interfering with endogenous transposon copies. For instance, the PiggyBac (PB) transposon originally isolated from the cabbage looper moth efficiently transposes cargo DNA into a variety of mammalian cells.

In the context of the present invention, vectors, in particular non-integrating vectors, may also be used for transient expression of a gene or a functional RNA. Transient expression is an expression for a limited amount of time and the time period of expression depends on the vector design and culturing conditions. However, transient expression means expression over a period of at least 24 hours but generally not more than 7 days.

Epigenetic regulatory elements can be used to protect the cargo DNA from unwanted epigenetic effects when placed near the transgene on plasmid vectors. For example, elements called matrix attachment region (MARs) were proposed to increase cargo DNA genomic integration and transcription while preventing heterochromatin silencing, as exemplified by the potent human MAR 1-68. They can also act as insulators and thereby prevent the activation of neighboring cellular genes. MAR elements have thus been used to mediate high and sustained expression in the context of plasmid or viral vectors. For transient gene expression, non-integrating vectors (sometimes referred to as episomal vectors) such as plasmids or non-integrating lentiviral (NIL) vectors may be used. They may be stably or transiently maintained and replicated within the host cell.

The vector sequence of a vector is the DNA or RNA sequence of the vector excluding any “other” nucleic acids such as transgenes as well as genetic elements such as MAR elements.

The term sequence Identity refers to a measure of the identity of nucleotide sequences or amino acid sequences. In general, the sequences are aligned so that the highest order match is obtained. “Identity”, per se, has a recognized meaning in the art and can be calculated using published techniques. (See, e.g.: Computational Molecular Biology, 30 Lesk, A. M., ed., Oxford University Press, New York, 1988; Biocomputing: Informatics and Genome Projects, Smith, D. W., ed., Academic Press. New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans as defining identical nucleotides or amino acids at a given position in the sequence (Carillo, H. & Lipton, D., SIAM J Applied Math 48:1073 (1988)).

Whether any particular nucleic acid molecule is at least 50%, 60%, 70%, 75%, 80%, 85%, 90%, 95%, 96%, 97%, 98% or 99% identical to. for instance, the gammaretrovirus-like sequences of SEQ ID NOs. 1, 2, 3, 4, 5 or a part thereof can be determined conventionally using known computer programs such as DNAsis software (Hitachi Software, San Bruno, Calif.) for initial sequence alignment followed by ESEE version 3.0 DNA/protein sequence software (cabot@trog.mbb.sfu.ca) for multiple sequence alignments.

Whether the amino acid sequence is at least 50%, 60%, 70%, 75%, 80%, 85%, 90%, 95%, 96%, 97%, 98% or 99% identical to, for instance a protein expressed by SEQ ID NOs:1, 2, 3, 4, 5 or a part thereof, can be determined conventionally using known computer programs such the BESTFIT program (Wisconsin Sequence Analysis Package®, Version 8 for Unix, Genetics Computer Group, University Research Park, 575 Science Drive, Madison, Wis. 53711). BESTFIT uses the local homology algorithm of Smith and Waterman, Advances in Applied Mathematics 2:482-489 (1981), to find the best segment of homology between two sequences.

When using DNAsis, ESEE, BESTFIT or any other sequence alignment program to determine whether a particular sequence is, for instance, 95% identical to a reference sequence according to the present invention, the parameters are set such that the percentage of identity is calculated over the full length of the reference nucleic acid or amino acid sequence and that gaps in homology of up to 5% of the total number of nucleotides in the reference sequence are allowed.

Another preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to Ts. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=0, Cutoff Score=1, Gap Penalty=5, Gap Size Penalty=0.05, Window Size=500 or the length of the subject nucleotide sequence, whichever is shorter.

For example, a polynucleotide having 95% “identity” to a reference nucleotide sequence of the present invention, is identical to the reference sequence except that the polynucleotide sequence may include on average up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence, the ORF (open reading frame), or any fragment specified as described herein.

The NCBI Basic Local Alignment Search Tool (BLAST) (Altschul et al. J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx. It can be accessed at the NCBI website, together with a description of how to determine sequence identity and sequence similarities using this program.

The invention is not only directly to sequence having a certain sequence identity with the sequences disclosed herein but is, equally, directed to sequence variants of any of the sequences disclosed herein. The invention is thus also directed to sequence variants in any context in which a certain sequence identity is mentioned and vice versa. A “sequence variant” refers to a polynucleotide or polypeptide differing from the sequences disclosed herein (polynucleotide or polypeptide sequences), but retaining essential properties thereof. Generally, variants are overall closely similar and in many regions, identical to the sequences disclosed herein.

The variants may contain alterations in the coding regions, non-coding regions, or both. Especially preferred are sequence variants containing alterations which produce silent substitutions, additions, or deletions, but do not alter the properties or activities of, e.g., the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred.

The invention also encompasses allelic variants of said polynucleotides. An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The amino acid sequences of the variant polypeptides may differ from the amino acid sequences depicted in SEQ ID NOS:1, 2, 3, 4 or 5 by an insertion or deletion of one or more amino acid residues and/or the substitution of one or more amino acid residues by different amino acid residues. Preferably, amino acid changes are of a minor nature, that is conservative amino acid substitutions that do not significantly affect, e.g., the folding and/or activity of the protein: small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions which do not generally alter the specific activity are known in the art and are described, for example, by H. Neurath and R. L. Hill, 1979. In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ille, Leu/Val, as well as these in reverse.

A certain percentile of “consecutive nucleotides” means nucleotides directly following each other. Thus 10% of the nucleotides of SEQ ID NO:2, which contains 60000 nucleotides could be nucleotide 1-6000 or nucleotide 2-6001 etc.

If a sequence is said to be “directly adjacent” to another, this means that there are no intervening sequences. Flanking regions are directly adjacent to a particular sequence and denotes 5′ and 3′ regions of a specific nucleic acid sequence.

Gene silencing via, e.g., siRNAs has been described elsewhere, for example in US Patent Publication 20180016583. which is incorporated herein by reference in its entirety, and specifically for its disclosure and gene silencing.

CHO cells are the most widely used expression system for therapeutic proteins, but also a recognized source of adventitious viral-like particles for more than 40 years (7-10). Although these particles were never shown to be infectious, their genomic origin and possible evolution remain mostly unknown. Thus, safety concerns have persisted, and ample precautions must be taken when purifying therapeutic proteins. Here, this issue was approached by characterizing CHO endogenous retroviral elements at the genome, transcriptome and viral particle level, showing that CHO cells are able to release intact viral particles loaded with viral RNA genomes of group 1 type-C ERVs. The sequence encodes a full-length open reading frame, thus likely producing functional viral proteins. This finding challenges the only available study on CHO viral particle sequences, published in 1994, in which the authors detected only defective DNA sequences with numerous mutations in the ERV genes (12). Using this updated viral particle RNA sequence, the number of possible ERV loci responsible for the expression and release of CHO viral particles was limited to a group of up to 30 well-conserved group 1 type-C ERV sequences in the CHO genome.

Next, the Myr and PPYP budding motifs of the functionally relevant group 1 type-C ERV sequences were mutated using CRISPR-Cas9, to seek to prevent ERV budding. After transient CRISPR-Cas9 expression, 10-15% of the isolated clones contained mutations in the expressed group 1 sequences, some of which causing Gag loss-of-function effects. Having introduced unique mutations into defined ERV sequences a single genomic ERV locus as the origin of viral type-C particle formation in CHO cells could be pinpointed. Most interestingly, site-specific mutagenesis of this particular locus was sufficient to avoid release of viral particle carrying the viral genomic RNA. This indicated that the other ERVs present in the CHO genome may be unable to complement the Gag loss-of-function, nor become reactivated upon CRISPR-Cas9 mutagenesis.

A common technical challenge for multi-locus genome editing is the presence of extensive DNA damage. This damage may be elicited by the multiple Cas9-induced DSBs, which usually activate p53 signaling and cause cell death (20, 47-50). The sgRNAs designed in this study were predicted to perfectly recognize roughly 60 distinct group 1 type-C ERV loci in the CHO genome, although only some of them should be transcribed and may thus be preferentially cleaved by Cas9. Indeed, CRISPR-Cas9 treated clones possessed between 1 to 14 different mutation sites following a single transient transfection, suggesting that CHO cells are able to handle the DNA damage response and repair of up to 14 separate DSBs. In comparison to primary cells where sometimes a single DSB break results in cell death (50), transformed cell lines such as CHO cells typically encounter higher levels of endogenous DNA damage, and they are more likely to be able to handle and survive multi-loci genome editing, as seen here (51). However, even in CHO cells, a drop in cell proliferation and/or viability following a rather mild transient treatment with ERV-targeting sgRNAs was observed, which correlated well with the predicted number of target sites. An elevated cytotoxicity might have prevented the isolation of even more highly mutated clones. This would explain why a recent study reporting the isolation of primary porcine cells containing mutations in up to 62 endogenous viral elements required anti-apoptotic treatments to suppress p53-mediated cell death (20).

Another challenge in multi-locus editing is the plurality of repetitive ERV sequences present in the CHO genome that could be used as template for HR (homologous recombination) repair, which may counteract efficient gene knock-out mediated by C-NHEJ (canonical non-homologous end-joining repair) and alt-EJ (alternative end-joining) repair pathways. In CHO cells, HR activity is believed to be rather low compared to other cell (52, 53). Typically, HR may precisely repair DSBs (double strand breaks), but imprecise repair outcomes also occur (54). Here it was found that roughly 10% of the analyzed repair junctions at both sgRNA sites contained HR-compatible signatures, such as templated insertions from other ERV loci. Thus, it was hypothesized that HR repair is active and possibly opposes efficient ERV mutagenesis.

The genome editing strategy used in this study aimed primarily at introducing Gag loss-of-function mutations that interfere with proper Gag protein synthesis and thereby prevent ERV budding. However, as the person skilled in the art will appreciate that loss of function mutations in the pol gene or env gene and/or in at least one of the LTRs can also be introduced by appropriate procedures. As expected, clones mutated in the expressed group 1 type-C ERV sequence showed unchanged mRNA expression levels of group 1 and group 2 ERVs (data not shown), while being strongly impaired in releasing encapsulated viral RNA. In addition, ERV-mutated clones did not consistently differ in cell growth, cell size or therapeutic protein production compared to control samples. Hence, the differences between clones may be clone-specific. Clonal variation is a common phenomenon when isolating clones from polyclonal populations and has even been noticed during clone subcloning (57, 58). Clone-specific variability arises not only from genetic heterogeneity between the clones, for instance due to the acquisition of random and/or CRISPR-derived mutations, exposure to different stress responses, notably during CRISPR treatment, but also from stochastic fluctuations in protein expression and/or epigenetic effects (49, 58, 59). Furthermore, the accumulation of untranslated or nonsense mRNAs as well as of truncated and usually dysfunctional proteins in the cell cytoplasm has been associated with unclear side effects (60).

The present disclosure shows that a functionally active ERV locus can be selectively mutated using group 1 type-C specific sgRNAs. This offers novel avenues to improve the safety profile of CHO cells and thereby substantially reducing the number of virus inactivation and removal steps needed for viral clearance during biopharmaceutical production. The finding that a single ERV locus may be responsible for ERV expression and viral particle release by CHO cells enables to excise the entire 10 kb long proviral genome using two site-specific sgRNAs, as it has been done for HIV-infected human cells (61). This approach for ERV mutagenesis might reduce the elicited DNA damage response, possibly avoiding the accumulation of defective ERV RNAs in the cytoplasm and/or other detrimental side-effect arising from the mutation of other elements of the CHO genome, and consequently leading to less confounded effects on-target phenotypes.

### MATERIALS AND METHODS

### Cell Culture

Suspension-adapted Chinese hamster ovary (CHO-K1) derived cells were maintained in serum-free HyClone SFM4CHO medium supplemented with HyClone Cell boost 5 supplement (GE HEALTHCARE), L-glutamine (GIBCO), HT supplement (GIBCO) and antibiotic-antimycotic solution (GIBCO). CHO cell viability was assessed by Erythrosin B dye (SIGMA-ALDRICH) and viable cell density and cell size were quantified using the LUNA-FL Dual Fluorescence Cell Counter (LOGOS BIOSYSTEMS). The cells were cultivated in 50 ml TubeSpin bioreactor tubes (TPP, Switzerland) at 37° C., 5% CO₂ in a humidified incubator with 180 rpm agitation rate and passed every 3-4 days.

### Plasmid Construction

The mammalian codon-optimized Streptococcus pyogenes Cas9 (SpCas9) nuclease expression plasmid JDS246 (ADDGENE plasmid #43861) (21) was used to introduce site-specific DSBs. The CRISPRseek R package (22) was applied to design single guide RNA (sgRNA) sequences that target the myristoylation (Myr) or PPYP motifs in the gag consensus sequence of group 1 ERVs.

Among all potential sgRNAs, three Myr (Myr2, Myr4, Myr8)- and five PPYP (PPYP5, PPYP6, PPYP7, PPYP13, PPYP20)-specific sgRNA sequences were selected as they mediate DSB cleavage no more than 25 bp apart from the target motif, and as they were predicted to have high sgRNA efficiency using various scoring tools (CRISPRseek, (22); Sequence Scan for CRISPR, (23); sgRNA scorer 1.0. (24)) (TABLE 1).

TABLE 1
Predicted number of ERV target sites for Myr- and PPYP-specific sgRNAs in the
CHO-K1 genome.

| | | | Number of | | | | |
|---|---|---|---|---|---|---|---|
| sgRNA | | PAM | mismatches allowed | | | | |
| name | sgRNA sequence (5′-3′) | sequence* | 0 | 1 | 2 | 3 | Total |
| Myr2 | TCCTAAGCCTAGAAACTATG | Canonical | 59 | 29 | 16 | 26 | 147 |
| | (SEQ ID NO: 7) | Non-canonical | — | — | 1 | 16 | |
| Myr4 | CATAGTTTCTAGGCTTAGGA | Canonical | 33 | — | — | 9 | 54 |
| | (SEQ ID NO: 8) | Non-canonical | — | — | 1 | 11 | |
| Myr8 | GAGTGTTAGGGACAAAGGAG | Canonical | 117 | 30 | — | 36 | 218 |
| | (SEQ ID NO: 9) | Non-canonical | — | — | 2 | 33 | |
| PPYP5 | GTTGGTTGATCTATTAACGG | Canonical | 61 | 30 | 12 | 5 | 114 |
| | (SEQ ID NO: 10) | Non-canonical | — | — | — | 6 | |
| PPYP6 | GCCACTGCCGCCCCCACCAG | Canonical | 55 | 16 | 9 | 36 | 133 |
| | (SEQ ID NO: 11) | Non-canonical | 1 | — | — | 16 | |
| PPYP7 | GCCCCCACCAGAGGCAGAAG | Canonical | 69 | 65 | 41 | 60 | 283 |
| | (SEQ ID NO: 12) | Non-canonical | 3 | 3 | 3 | 39 | |
| PPYP13 | GGCAGTGGCGGATATGGCGG | Canonical | 58 | 16 | 14 | 42 | 142 |
| | (SEQ ID NO: 13) | Non-canonical | 1 | 2 | 1 | 8 | |
| PPYP20 | GCTTCTGCCTCTGGTGGGGG | Canonical | 70 | 63 | 8 | 47 | 217 |
| | (SEQ ID NO: 14) | Non-canonical | 3 | 4 | 5 | 17 | |

*The canonical PAM sequence of SpCas9 is NGG

Genome-wide off-target cleavage analysis for these sgRNA sequences was performed using the CRISPRseek R® package using the CHO-K1 cell genome as reference sequence. sgRNA oligonucleotides were designed using the Zinc Finger TARGETER software support tool (25, 26), and annealed sgRNA oligonucleotides were subsequently cloned into the mammalian sgRNA expression vector MLM3636 (ADDGENE plasmid #43860) as previously described (21). For sgRNA sequences lacking a guanine (G) nucleotide at the 5′ end, an additional, non-pairing G was appended to improve transcription from the sgRNA expression plasmid (27). All primers used were purchased from MICROSYNTH AG (Balgach, Switzerland) and are listed in TABLE 2.

TABLE 2A
Sequences of the sgRNAs and corresponding primers used in this study.

| sgRNA | | 5′-3 | Addition of G at |
|---|---|---|---|
| name | Orientation | target site (without PAM) | 5′ end for better U6 expression |
| Myr2 | Forward | TCCTAAGCCTAGAAACTATG | GTCCTAAGCCTAGAAACTATG |
| | strand | (SEQ ID NO: 7) | (SEQ ID NO: 15) |
| | | Oligo 1 | Oligo 2 |
| | | ACACCGTCCTAAGCCTAGAAACTATGG | AAAACCATAGTTTCTAGGCTTAGGACG |
| | | (SEQ ID NO: 16) | (SEQ ID NO: 17) |
| Myr4 | Reverse | CATAGTTTCTAGGCTTAGGA | GCATAGTTTCTAGGCTTAGGA |
| | strand | (SEQ ID NO: 8) | (SEQ ID NO: 18) |
| | | Oligo 1 | Oligo 2 |
| | | ACACCGCATAGTTTCTAGGCTTAGGAG | AAAACTCCTAAGCCTAGAAACTATGCG |
| | | (SEQ ID NO: 19) | (SEQ ID NO: 20) |
| Myr8 | Reverse | GAGTGTTAGGGACAAAGGAG | — |
| | strand | (SEQ ID NO: 9) | |
| | | Oligo 1 | Oligo 2 |
| | | ACACCGAGTGTTAGGGACAAAGGAGG | AAAACCTCCTTTGTCCCTAACACTCG |
| | | (SEQ ID NO: 21) | (SEQ ID NO: 22) |
| PPYP5 | Forward | GTTGGTTGATCTATTAACGG | — |
| | strand | (SEQ ID NO: 10) | |
| | | Oligo 1 | Oligo 2 |
| | | ACACCGTTGGTTGATCTATTAACGGG | AAAACCCGTTAATAGATCAACCAACG |
| | | (SEQ ID NO: 23) | (SEQ ID NO: 24) |
| PPYP6 | Forward | GCCACTGCCGCCCCCACCAG | — |
| | strand | (SEQ ID NO: 11) | |
| | | Oligo 1 | Oligo 2 |
| | | ACACCGCCACTGCCGCCCCCACCAGG | AAAACCTGGTGGGGGCGGCAGTGGC |
| | | (SEQ ID NO: 25) | G |
| | | | (SEQ ID NO: 26) |
| PPYP7 | Forward | GCCCCCACCAGAGGCAGAAG | — |
| | strand | (SEQ ID NO: 12) | |
| | | Oligo 1 | Oligo 2 |
| | | ACACCGCCCCCACCAGAGGCAGAAGG | AAAACCTTCTGCCTCTGGTGGGGGCG |
| | | (SEQ ID NO: 27) | (SEQ ID NO: 28) |
| PPYP13 | Reverse | GGCAGTGGCGGATATGGCGG | — |
| | strand | (SEQ ID NO: 13) | |
| | | Oligo 1 | Oligo 2 |
| | | ACACCGGCAGTGGCGGATATGGCGGG | AAAACCCGCCATATCCGCCACTGCCG |
| | | (SEQ ID NO: 29) | (SEQ ID NO: 30) |
| PPYP20 | Reverse | GCTTCTGCCTCTGGTGGGGG | — |
| | strand | (SEQ ID NO: 14) | |
| | | Oligo 1 | Oligo 2 |
| | | ACACCGCTTCTGCCTCTGGTGGGGGG | AAAACCCCCCACCAGAGGCAGAAGCG |
| | | (SEQ ID NO: 31) | (SEQ ID NO: 32) |

TABLE 2B
Sequences of the PCR and Illumina sequencing primers used to
characterize corresponding genomic loci of edited CHO cells.

| | | |
|---|---|---|
| Primer Name | Full sequence (5′-3′) | SEQ ID NO: |

Myr Forward Primers

| | | |
|---|---|---|
| Myr_Fa_3 | TCGTCGGCAGCGTCAGATGTGTATAAGAGACAGACCGCTTGAAGGATTTGCAATC | 33 |
| Myr_Fb_0 | TCGTCGGCAGCGTCAGATGTGTATAAGAGACAGGCTTGAGGGATTTGCAATC | 34 |
| Myr_Fb_1 | TCGTCGGCAGCGTCAGATGTGTATAAGAGACAGTGCTTGAGGGATTTGCAATC | 35 |
| Myr_Fb_2 | TCGTCGGCAGCGTCAGATGTGTATAAGAGACAGTTGCTTGAGGGATTTGCAATC | 36 |
| Myr_Fb_3 | TCGTCGGCAGCGTCAGATGTGTATAAGAGACAGACTGCTTGAGGGATTTGCAATC | 37 |
| Myr_Fc_2 | TCGTCGGCAGCGTCAGATGTGTATAAGAGACAGGTGCTTGAGGGATTTGTAATC | 38 |

Myr Reverse Primers

| | | |
|---|---|---|
| Myr_R_0 | GTCTCGTGGGCTCGGAGATGTGTATAAGAGACAGACAAAGAGTAATCCATTTGCG | 39 |
| Myr_R_1 | GTCTCGTGGGCTCGGAGATGTGTATAAGAGACAGGACAAAGAGTAATCCATTTGCG | 40 |
| Myr_R_2 | GTCTCGTGGGCTCGGAGATGTGTATAAGAGACAGCGACAAAGAGTAATCCATTTGCG | 41 |
| Myr_R_3 | GTCTCGTGGGCTCGGAGATGTGTATAAGAGACAGAAGACAAAGAGTAATCCATTTGCG | 42 |

PPYP Forward Primers

| | | |
|---|---|---|
| PPYP_Fa_0 | TCGTCGGCAGCGTCAGATGTGTATAAGAGACAGACTCCAGCCTTTACCCTAC | 43 |
| PPYP_Fa_1 | TCGTCGGCAGCGTCAGATGTGTATAAGAGACAGAACTCCAGCCTTTACCCTAC | 44 |
| PPYP_Fb_0 | TCGTCGGCAGCGTCAGATGTGTATAAGAGACAGATTCCAACCTTTACCCTAC | 45 |
| PPYP_Fb_1 | TCGTCGGCAGCGTCAGATGTGTATAAGAGACAGGATTCCAACCTTTACCCTAC | 46 |
| PPYP_Fb_2 | TCGTCGGCAGCGTCAGATGTGTATAAGAGACAGTGATTCCAACCTTTACCCTAC | 47 |
| PPYP_Fb_3 | TCGTCGGCAGCGTCAGATGTGTATAAGAGACAGCTTATTCCAACCTTTACCCTAC | 48 |

PPYP Reverse Primers

| | | |
|---|---|---|
| PPYP_Ra_1 | GTCTCGTGGGCTCGGAGATGTGTATAAGAGACAGGGGTCTGATGCTGAGAATG | 49 |
| PPYP_Rb_0 | GTCTCGTGGGCTCGGAGATGTGTATAAGAGACAGGGTCCGATGCTGAGAATG | 50 |
| PPYP_Rb_1 | GTCTCGTGGGCTCGGAGATGTGTATAAGAGACAGTGGTCCGATGCTGAGAATG | 51 |
| PPYP_Rb_2 | GTCTCGTGGGCTCGGAGATGTGTATAAGAGACAGGTGGTCCGATGCTGAGAATG | 52 |
| PPYP_Rb_3 | GTCTCGTGGGCTCGGAGATGTGTATAAGAGACAGAAGGGTCCGATGCTGAGAATG | 53 |

| | | | SEQ ID | |
|---|---|---|---|---|
| Ilumina Adapter | Spacer | Gene-specific Primer | NO: | PR |
| TCGTCGGCAGCGTCAGATGTGTATAAGAGACAG | ACC | GCTTGAAGGATTTGCAATC | 33 | 0.15 |
| TCGTCGGCAGCGTCAGATGTGTATAAGAGACAG | | GCTTGAGGGATTTGCAATC | 34 | 0.2 |
| TCGTCGGCAGCGTCAGATGTGTATAAGAGACAG | T | GCTTGAGGGATTTGCAATC | 35 | 0.2 |
| TCGTCGGCAGCGTCAGATGTGTATAAGAGACAG | TT | GCTTGAGGGATTTGCAATC | 36 | 0.2 |
| TCGTCGGCAGCGTCAGATGTGTATAAGAGACAG | ACT | GCTTGAGGGATTTGCAATC | 37 | 0.2 |
| TCGTCGGCAGCGTCAGATGTGTATAAGAGACAG | GT | GCTTGAGGGATTTGTAATC | 38 | 0.05 |
| | | | | 1 |
| GTCTCGTGGGCTCGGAGATGTGTATAAGAGACAG | | ACAAAGAGTAATCCATTTGCG | 39 | 0.25 |
| GTCTCGTGGGCTCGGAGATGTGTATAAGAGACAG | G | ACAAAGAGTAATCCATTTGCG | 40 | 0.25 |
| GTCTCGTGGGCTCGGAGATGTGTATAAGAGACAG | CG | ACAAAGAGTAATCCATTTGCG | 41 | 0.25 |
| GTCTCGTGGGCTCGGAGATGTGTATAAGAGACAG | AAG | ACAAAGAGTAATCCATTTGCG | 42 | 0.25 |
| | | | | 1 |
| TCGTCGGCAGCGTCAGATGTGTATAAGAGACAG | | ACTCCAGCCTTTACCCTAC | 43 | 0.1 |
| TCGTCGGCAGCGTCAGATGTGTATAAGAGACAG | A | ACTCCAGCCTTTACCCTAC | 44 | 0.1 |
| TCGTCGGCAGCGTCAGATGTGTATAAGAGACAG | | ATTCCAACCTTTACCCTAC | 45 | 0.2 |
| TCGTCGGCAGCGTCAGATGTGTATAAGAGACAG | G | ATTCCAACCTTTACCCTAC | 46 | 0.2 |
| TCGTCGGCAGCGTCAGATGTGTATAAGAGACAG | TG | ATTCCAACCTTTACCCTAC | 47 | 0.2 |
| TCGTCGGCAGCGTCAGATGTGTATAAGAGACAG | CTT | ATTCCAACCTTTACCCTAC | 48 | 0.2 |
| | | | | 1 |
| GTCTCGTGGGCTCGGAGATGTGTATAAGAGACAG | G | GGTCTGATGCTGAGAATG | 49 | 0.04 |
| GTCTCGTGGGCTCGGAGATGTGTATAAGAGACAG | | GGTCCGATGCTGAGAATG | 50 | 0.24 |
| GTCTCGTGGGCTCGGAGATGTGTATAAGAGACAG | T | GGTCCGATGCTGAGAATG | 51 | 0.24 |
| GTCTCGTGGGCTCGGAGATGTGTATAAGAGACAG | GT | GGTCCGATGCTGAGAATG | 52 | 0.24 |
| GTCTCGTGGGCTCGGAGATGTGTATAAGAGACAG | AAG | GGTCCGATGCTGAGAATG | 53 | 0.24 |
| | | | | 1 |

PR = Primer ratio acc. to expected % of ERVs (total = 1)

TABLE 2C
Sequences of the PCR and qPCR primers used to characterize and validate
ERV loci and expression

| Amplification type | Oligo 1 | Oligo 2 |
|---|---|---|
| Locus ERV Type 1 | CTCTGGTTCTTGCCTGCTGAGCT | TGGTCAATGTATATGAGGCGCT |
| validation1 | (SEQ ID NO: 54) | (SEQ ID NO: 55) |
| qPCR Type1 ERV | GGGAATTGAGTCTGCTGTACCA | ACAGAGTCTTTCAAATGAGGCG |
| specific LTR | (SEQ ID NO: 56) | (SEQ ID NO: 57) |
| qPCR ref GAPDH | GCGACTTCAACAGTGACTCCCA | TGAGGTCCACCACTCTGTTGCT |
| | (SEQ ID NO: 58) | (SEQ ID NO: 59) |
| qPCR Type2 ERV | GAATAAAAGGTCAGGGCGTTGG | CTGACTTGGCTCTATCTTGGGT |
| specific | (SEQ ID NO: 60) | (SEQ ID NO: 61) |
| qPCR Type1 ERV | TGACGATATAAGCCACTTGA | ACCCCCAGACTATATTCCAGATA |
| specific Gag | (SEQ ID NO: 62) | (SEQ ID NO: 63) |
| qPCR Type1 ERV | CTATGTGCTGCCCTCAAGGA | GCCTCTCCCTAAGTTTGGCC |
| specific Env | (SEQ ID NO: 64) | (SEQ ID NO: 65) |
| Locus ERV type 1 | CTCTGGTTCTTGCCTGCTGAGCT | TAAGCCATTGGTGAAGGGTCA |
| validation2 | (SEQ ID NO: 54) | (SEQ ID NO: 66) |
| Locus ERV type 1 | CTCTGGTTCTTGCCTGCTGAGCT | TGACGATATAAGCCACTTGA |
| validation3 | (SEQ ID NO: 54) | (SEQ ID NO: 62) |
| Locus ERV type 1 | TTTTCTGGTGCCCTCTTGCCTGG | TAAGCCATTGGTGAAGGGTCA |
| validation4 | (SEQ ID NO: 67) | (SEQ ID NO: 66) |
| Locus ERV type 1 | CTCTGGTTCTTGCCTGCTGAGCT | TTGTGGAGCTGTGTGAGTGGTGG |
| validation without ERV | (SEQ ID NO: 54) | (SEQ ID NO: 68) |

RNA Extraction from VP and VIP

Total RNA was extracted from the VPs and VLPs isolated CHO culture supernatants using the Invitrogen PureLink® Viral RNA/DNA mini kit (THERMO FISHER SCIENTIFIC) according to the manufacturer's protocol with some modifications. The supernatants were used freshly prepared, or after only one freezing and thawing cycle. 500 μl of supernatant was loaded on a Corning Costar Spin-X column centrifuge tube with 0.22 μm membrane filter and centrifuged at 16000 g for 1 minute. Approximately 12.5 units of RNase free DNase (MACHEREY-NAGEL) were added to 5000± of CHO cell culture supernatants, which were incubated for 15 min at 37° C. to digest the residual DNA possibly present. The resulting extracts were then treated as described in the PureLink® Viral RNA/DNA mini kit protocol. The RNA recovered from the spin columns was resuspended in 300 μl of RNase free water, followed by another DNase treatment using 10 units of RNase free DNase (MACHEREY-NAGEL) for 30 min at 37° C. After the addition of EDTA at a 5 mM final concentration, a DNase denaturation step was made by incubating the extracts at 70° C. for 15 min. The samples were after place on a MICRODIALYSIS MF-MILLIPORE Membrane Filter (MERK-MILLIPORE) type VSWP 0.025 μm pore for 15 min in order to remove salts such as EDTA remaining in the samples.

### Inactivation of ERV Sequences, Fluorescent Cells Enrichment and Single Cell Isolation

CHO-K1 cells were seeded at 300,000 cells/ml one day prior to transfection. On the day of transfection, 700,000 cells were electroporated with 3700 ng of CRISPR-Cas9 and 1110 ng of Myr- or PPYP-specific sgRNA expression plasmids using the NEON transfection system (THERMO FISHER SCIENTIFIC), according to the manufacturers instructions. CRISPR-Cas9 and sgRNA expression plasmids were used at equimolar ratio. 200 ng of pCMV-DsRed-Express plasmid (CLONETECH) was added to each transfection condition as transfection control. For CRISPR control experiments, the Myr or PPYP-specific sgRNA plasmids were substituted with the empty sgRNA expression vector (empty vector control).

To enrich for transfected and ERV mutated CHO cells, at least 70,000 cells were bulk-sorted for the highest 30-40% of transfected dsRed expressing cell population 48-72h after transfection using the MOFLO ASTRIOS EQ or FACSAria II cell sorters (BECKMAN COULTER). Cells were then briefly centrifuged to exchange medium and expanded. To isolate single cell clones, CRISPR-treated cells were incubated at room temperature with DAPI viability dye (BD BIOSCIENCES) for 15 min. Viable cells were single cell sorted into 96well plates using the FACSAria Fusion cell Sorter® (BECKMAN COULTER). Cell clones were recovered in HyClone® SFM4CHO medium supplemented with L-glutamine, HT supplement, antibiotic-antimycotic solution and ClonaCell-CHO ACF Supplement (STEMCELL TECHNOLOGIES) to increase post-sort survival. Flow cytometry data were analyzed using FlowJo® software v10.4.2. Cells were first gated using side scatter (SSC) versus forward scatter (FSC) to separate the intact cell population from debris, and then selected for single cells in the SSC-H/SSC-W and FSC-H/FSC-W plots. This single cell population was then gated for dsRed+ cells with non-fluorescent cells as gating control.

### ERV Mutation Efficiency

To assess the cleavage efficiency of ERV-specific sgRNAs, the frequency of ERV mutations was determined among the transcribed ERV sequences. Total RNA from CRISPR-treated polyclonal cell populations was extracted using the NUCLEOSPIN RNA kit (MACHEREY NAGEL) and reverse transcribed into cDNA using oligo(dT)₁₅ primers and the GoScript® Reverse Transcription System (PROMEGA). For CRISPR-treated single cell clones, total RNA was isolated using the SV 96 Total RNA Isolation System (PROMEGA) and reverse transcribed using GoScript® Reverse Transcription Mix, Oligo(dT) (PROMEGA). PCR amplification of the CRISPR target regions was carried out using OneTaq® DNA polymerase (NEW ENGLAND BIOLABS) with group 1 ERV-specific primers (TABLE 2B). PCR products were analyzed by Sanger sequencing and analyzed for mutations. The mutagenesis frequency in CRISPR-treated polyclonal populations was determined by decomposition of the mixed Sanger sequencing chromatograms and comparison to untreated (wild-type) cells using the TIDE software (28).

### Deep Amplicon Sequencing Analysis of CRISPR-Targeted Genomic Regions

To assess the number of CRISPR-induced ERV mutations at the genome level, DNA was extracted from ERV-edited CHO clones using the DNeasy Blood & Tissue Kit® (QIAGEN). This extracted genomic DNA was used to prepare sequencing libraries in a two-step PCR approach as described in the Illumina “16S Metagenomic Sequencing Library Preparation” protocol with some modifications. Briefly, degenerate primers were designed using the Primer Design-M tool (29) to amplify approximately 300 bp of the genomic region flanking the Myr2 and PPYP6 sgRNA target sites of all predicted type-C ERV sequences (290 bp amplicon for Myr, 314 bp amplicon for PPYP, TABLE 2). Degenerate primers contained various 0-3 bp heterogeneity spacers to increase template complexity (30) and Myr or PPYP primers were mixed at the predicted genomic frequency. In the first PCR round, 100 ng of isolated genomic DNA was used to PCR amplify the Myr and PPYP target loci using KAPA HiFi HotStart ReadyMix® (2×) (KAPA BIOSYSTEMS) for 23 and 20 cycles, respectively. PCR amplicons were purified with AMPure XP® beads (BECKMAN COULTER) using a 1:1 bead ratio. Amplicon quality and size were verified on an Agilent 2100 Bioanalyzer® and DNA was quantified using the Qubit dsDNA HS Assay Kit® (THERMO FISHER SCIENTIFIC). In the second PCR round, Illumina Nextera XT Index® sequencing adapters were added to 15 ng of purified amplicons using 8 PCR cycles. The final libraries were purified with AMPure XP® beads (BECKMAN COULTER) using a 1:1.12 bead ratio. Library quality and size were verified using Fragment Analyzer (ADVANCED ANALYTICAL) and quantified using Qubit dsDNA HS Assay Kit® (THERMO FISHER SCIENTIFIC). Libraries were pooled at equimolar ratio, spiked with 25% PhiX and sequenced using 2×250 bp paired-end sequencing on an Illumina Miseq System® at the Genomic Technologies Facility of the University of Lausanne (Switzerland).

For all identified mutations, Illumina raw reads were clustered using the Jukes-Cantor genetic distance model under the UPGMA tree building method to test for ERV locus-specific genetic variations in the mutation flanking region.

### Whole Genome Sequencing of ERV-Mutated CHO Clone

To identify mutated ERV loci in the whole CHO genome, high-molecular-weight DNA was extracted from the sgRNA PPYP6-treated E10 clone using the blood & cell culture DNA kit (QIAGEN). DNA quality and quantity were verified using Fragment Analyzer (ADVANCED ANALYTICAL) and Quibit® (THERMO FISHER SCIENTIFIC), respectively. Sample sequencing was performed on a PacBio Sequel System® (PACIFIC BIOSCIENCES) at the Genomic Technologies Facility of the University of Lausanne (Switzerland).

### Analysis of Therapeutic Protein Expression

To assess the therapeutic protein production capacity of ERV-modified cells, polyclonal cell populations and cell clones previously treated with ERV-specific or empty sgRNA expression plasmids were electroporated with a trastuzumab immunoglobulin G1 (IgG1) heavy and light chain expression vector bearing a puromycin resistance gene (31). As control, wild-type CHO-K1 cells were transfected with the same expression vector in parallel. Two days after transfection, cells were transferred to culture medium containing 5 μg/ml puromycin and selected for three weeks.

Immunoglobulin titers from cultures of stable trastuzumab expressing cell populations were quantified during ten-days fed-batch cultures as previously described (31). Briefly, cells were seeded at 0.3*10⁶ cells/ml in 5 ml initial culture volume without Puromycin selection. Cell culture was fed with HyClone® Cell boost 5 supplement (GE HEALTHCARE) at 16% of the initial culture volume on days zero, two, three and six to eight of the cell cultivation. Cell density and viability was assessed at days three, six, eight and ten and immunoglobulin secretion in the cell culture supernatant was measured on days six, eight and ten by sandwich ELISA.

### Characterization of ERV Elements in CHO-K1 Cells

To search for ERVs present in CHO cells, the CHO-K1 genome was assembled de novo using PacBio® long-read sequencing, and the previously reported IAP and ML2G murine retroviral sequences were searched in this assembly (12, 13). Furthermore, we used as well profiles to complement and validate the ERV elements identified by sequence similarity. Approximately 160 copies of IAP-like proviral elements were found within the CHO genome. In addition to approximately 200 IAPs, 173 gammaretrovirus type-C proviruses were identified that shared at least 80% sequence identity to the ML2G sequence in CHO cells (12) (TABLE 3).

TABLE 3
Number and frequency of distinct type-C ERV sequences detected in
the genome, transcriptome and viral particles of CHO-K1 cells.

| | Type-C ERV | ERV group relative |
|---|---|---|
| Detection level | sequence number | frequency |
| Genomic DNA | 173 | group 1 ≅ group 2 |
| Cellular mRNA | 3-32 | group 1 > group2 |
| Viral particles | 1-5 | group 1 only |

Although the identified number of type-C proviruses was in line with previous estimations (6). it was noticed that some ERV copies could not be successfully placed in the assembly suggesting that 173 copies is likely an underestimation of the total reservoir of type-C ERV elements in CHO cells. Among the identified 173 type-C ERV sequences, only 112 contained the gag, pol and env genes, as required to produce a functional ERV. Phylogenetic analysis of these full-length hamster type-C ERV sequences revealed their close similarity to other mammalian retroviral elements, such as the Feline leukemia virus (FeLV) and the Murine leukemia virus (MLV) (data not shown). Among these type-C ERV sequences, we identified two distinct groups: group 1 and group 2 which were composed of 101 and 36 members, respectively (FIG. 1A). Group 1 and group 2 type-C ERVs formed the predominant and functionally most conserved sequence clusters, with complete 5′ LTR-gag-pol-env-3′ LTR proviral structures, and they also shared most similarity to MLV elements, which are known to produce viral particles infecting primate cell lines (16). This implied the ERVs of group 1 and 2 as the most likely candidates for viral particle formation.

Further sequence analysis highlighted that the gag and pol genes were highly conserved among group 1 and group 2 ERV sequences but that ERVs belonging to group 1 showed overall less diversity than ERVs from group 2 (FIG. 1B-D), and revealed the presence of a possibly distinct and less conserved third group of ERVs, for instance from Env-based phylogeny (FIG. 1D). On average group 1 ERV sequences shared 99% sequence identity and likely form three subgroups (marked in different shades in FIG. 1B-D). However, the overall high conservation of these ERV sequences and the frequency of sequencing errors in genomes assembled using PacBio® reads hampered the direct identification of which of these group 1, 2 and group 3 ERVs may be functional and potentially active.

To complement the genomic CHO ERV characterization, the total cellular mRNA was sequenced using Illumina Short-read® technology to refine the transcribed ERV sequences. Type-C ERV mRNAs were among the top 10 most abundant transcripts in CHO cells (data not shown). Mapping of these Illumina® reads to type-C ERV representatives showed that 99.5% of all reads had sequences corresponding to group 1 and 2, indicating that these two groups contribute the vast majority of the transcribed ERVs of CHO cells. While the Illumina® reads mapped mainly on two easily distinguishable group 2 ERV sequences, they mapped on approximately 30 group 1 ERV sequences (FIG. 2A). As group 1 ERVs are most highly conserved, this did not allow unambiguous attribution of these reads to one or few unique group 1 loci. Interestingly, both transcribed group 2 ERV sequences contained interrupted ORFs and/or missing coding sequences, one containing a deletion of 2350 bp in the pol gene and the second having one frameshift in the gag and pol genes, as well as three stop codon mutations in pol. These mutations were confirmed by Sanger sequencing. In contrast, the transcribed group 1 ERV sequences seemed to encode full-length gag, pol and env transcripts. Overall, this suggested that between 3 to 32 ERV loci are transcribed, corresponding to approximately 2-20% of the total ERV elements in CHO cells (TABLE 3). Such an ERV expression frequency agrees with previous reports indicating that the majority of endogenized ERVs are epigenetically silenced in cell lines and organisms (32). Finally, among the total cellular mRNA, LTR-containing viral genomic RNA was also detected, indicating that CHO cells are capable of producing retroviral genomes that may be encapsulated and released as retroviral particles in the cell supernatant.

Retroviral-like particles released by cultured CHO cells were isolated, and the viral genomic RNA sequences were extracted and characterized by deep-sequencing using Illumina® technology. A twenty-fold enrichment in LTR-containing viral genomic RNA was observed when compared to the total cellular mRNA sequences (FIG. 2A). This indicated that CHO cells are able to shed retroviral particles containing genomic viral RNA into the cell supernatant. In-depth analysis of these viral RNA sequences indicated that group 1-derived reads were mostly present in the released viral particles (FIG. 2B). Moreover, these sequences could be mapped to just 1 to 5 different group 1 ERV sequences, suggesting that only few group 1 ERV loci are responsible for the production of viral particles (VPs) in CHO cells (TABLE 3).

To further characterize the functional group 1 type-C ERV sequences, group 1-specific probes for Fluorescent in-situ hybridization (FISH) experiments were designed. Using these probes, approximately 50-100 group 1 ERV integration sites in the CHO-K1 genome 20 were detected, in line with the number of viral integration events detected in the newly assembled genome (FIG. 2C and TABLE 3). Retroviral integrations were dispersed throughout the CHO-K1 genome, with a possible integration hotspot in one of the smallest chromosomes. Additionally, when staining for group 1 nascent mRNAs, a unique highly transcribed site, suggesting that only a single group 1 ERV locus might be transcriptionally active, was observed (FIG. 2D).

Altogether, systematic ERV characterization at the genome, transcriptome and viral particle (VP) level identified several type-C group 1 ERVs as strong candidates for the expression and release of functional retroviral particles from CHO-K1 cells. Although the high sequence identity among the type-C ERV sequences concealed the exact number of expressed ERV loci, these data suggested that mutating few transcribed group 1 ERV loci by genome editing might suffice to prevent ERV particle formation.

Designing ERV-Specific sgRNA Sequences for CRISPR-Cas9 Genome Editing

To inhibit the release of potentially infective viral particles (VPs) from CHO cells, it was the aim to disrupt conserved ERV sequence motifs critical for VP release. The Gag protein plays a pivotal role during retrovirus budding, and, consistently, it was conserved among the type-C ERVs in CHO cells. However, in contrast to the pol gene for instance, the gag sequences were sufficiently different to distinguish group 1 from group 2 type-C ERV sequences, allowing to specifically target group 1 ERV particles (FIGS. 1B and 1C). Two conserved gag sequences involved in viral budding were selected—the myristoylation (Myr) and the PPxY motifs—as targets for CRISPR-Cas9-mediated mutagenesis. The N-terminal Myr motif locates at a glycine residue at position 2 downstream of the ATG translation initiation codon (FIG. 3). Myristoylation of Gag is generally considered essential for targeting the protein to the host plasma membrane (33). Mutations that directly interfere with Gag myristoylation, that block translation from the physiological start site or that create a loss-of-function gag transcript will perturb proper viral particle assembly at the plasma membrane, and hence block retroviral particle budding (33, 34).

In addition to Myr, the conserved proline-rich PPxY motif also contributes to retrovirus budding, likely by interacting with the ESCRT machinery (35). and its mutation strongly inhibits viral particle release (36). The PPxY motif overlapped with a PPYP motif that is conserved in group 1 and group 2 CHO ERVs. which is termed PPYP hereafter to refer to this CHO-specific PPxY-related budding motif.

Eight sgRNAs against the group 1 gag consensus sequence were designed: three constructs targeting the Myr motif (Myr2, Myr4, Myr8) and five constructs targeting the PPYP motif (PPYP5, PPYP6, PPYP7, PPYP13, PPYP20) (FIG. 3). The selected sgRNA sequences located close to the corresponding target motifs and were predicted to perfectly match between 33 and 117 target ERV sequences, but to target up to 283 sites when allowing a maximum of three mismatches and non-canonical Protospacer adjacent motif (PAM) sites (TABLE 1). Importantly, all these potential cleavage sites map to ERV sequences, while other off-target sites in the CHO genome were not detected. Although these sgRNA sequences contain a multitude of predicted target sites, it was hypothesized that expressed ERVs might be preferentially cleaved by the CRISPR-Cas9 nuclease, due its preference for open chromatin (37).

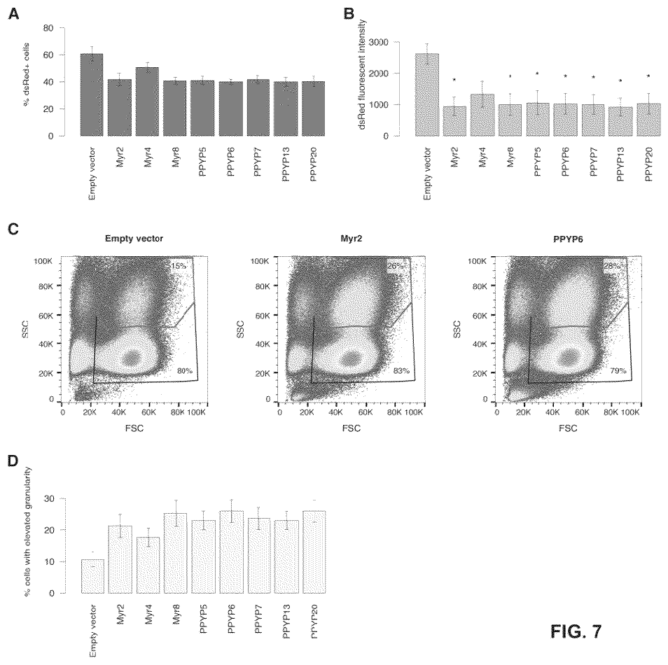

To mutate the Gag budding motifs, CHO-K1 parental cells were transiently transfected with CRISPR-Cas9 and Myr or PPYP sgRNA expression plasmids together with a dsRed transfection control plasmid. For CRISPR control samples, the gag-specific sgRNA expression plasmids were replaced with a non-targeting empty vector sgRNA control plasmid (empty vector) or left untreated (wild-type). Transfected dsRed positive (dsRed+) cells were bulk-sorted to enrich for cells containing mutations in the target motifs. Following treatments with ERV-specific sgRNAs, an overall reduced frequency of transfected dsRed+ cells as well as a significant drop in dsRed fluorescence intensity in dsRed+ cells compared to control samples were noted, suggesting that the most highly transfected cells may not survive because of a high frequency of genome cleavage (FIGS. 7A and B). Consistently, this effect was reduced for Myr4 sgRNA treated cells, which has the lowest number of predicted target sites. We also observed an elevated cell granularity following CRISPR treatment which inversely correlated with the frequency and expression intensity of dsRed+ cells (FIGS. 7C and D). Highly granular cells were previously reported to consist of pro-apoptotic and/or dying cell populations (38). Altogether, this provides evidence that CRISPR-mediated ERV cleavage impedes cell proliferation and survival, especially in highly transfected cells, implying that ERV-specific sgRNAs efficiently introduce DSBs at multiple target sites in the CHO genome.

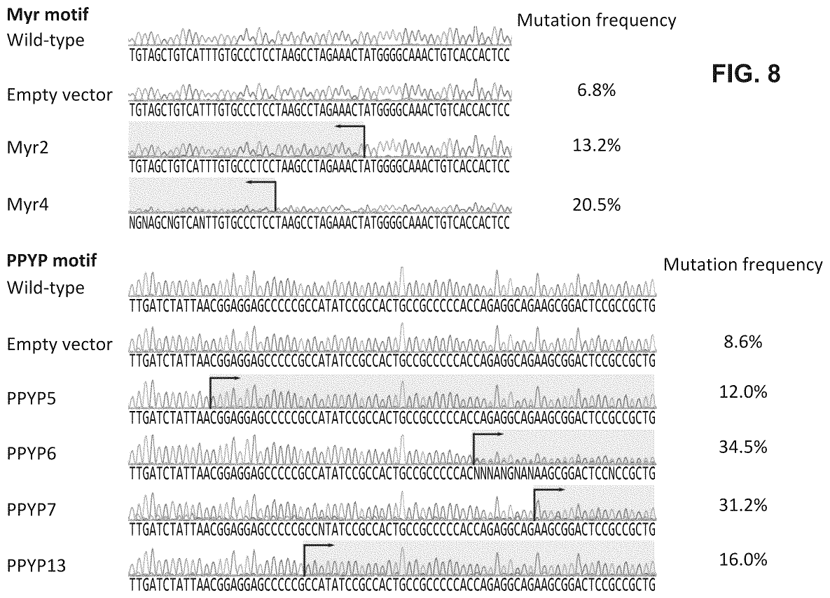

To estimate the CRISPR-mediated mutagenesis frequency within the expressed group 1 ERVs, the total cellular mRNA of bulk-sorted Myr- and PPYP-treated cells was reverse transcribed and PCR amplified, followed by the direct sequencing of the polyclonal PCR products or by their cloning into bacterial vectors prior to single colony sequence analysis. Based on these analyses, it was estimated that the designed gag-specific sgRNAs introduced mutations in roughly 9 to 35% of the ERV mRNAs, and that the Myr2 or PPYP6 sgRNAs were most efficient (FIG. 8, TABLE 4, TABLE 5). Interestingly, some of the recovered mutations were expected to block translation or introduce frameshifts, and thus should cause Gag loss-of-function phenotypes.

TABLE 4
Detection of CRISPR-mediated mutations in expressed
type-C ERV sequences cloned into plasmid vectors.

| | | | | Loss-of-function |
|---|---|---|---|---|
| | Analyzed | Mutated | Mutation | mutation |
| Sample | sequence | sequences | frequency | frequency (1) |
| Myr2 sgRNA | 12 | 2 | 17% | 50% |
| PPYP6 sgRNA | 56 | 4 | 7% | 75% |
| PPYP13 sgRNA | 12 | 1 | 8% | 0% |
| Total | 80 | 7 | 9% | 42% |

(1) Includes translation inhibition and frameshift mutations and is expressed relative to the number of mutated sequences.

TABLE 5 shows mRNA Sanger sequencing data of the expressed ERV repair junctions of CHO-K1 cells treated with wild-type Cas9 nuclease and various sgRNAs (Myr2, PPYP6 and PPYP13). The sequences are derived from Sanger sequencing of cDNA PCR amplicons cloned into plasmid vectors.

TABLE 5
Sequence analysis of ERV-mutated CHO-K1 mRNA junction amplicons cloned
into plasmid vectors.

| | | | Mutation | Repair | Pattern | FORE |
|---|---|---|---|---|---|---|
| | | Features | type | pathway | score | CasT |
| Sample (1) | Sequence (2) | (3) | (4) | (5) | (6) | (7) |

Myr2 sgRNA (n = 2)

| | | | | | | | |
|---|---|---|---|---|---|---|---|
| 4_5 | Genomic | 5′-CTGTCATTTG | 1 bp | Outside ERV | C-NHEJ | — | 17.8% |
| | | TGCCCTCCTAAGC | insertion, 1 | coding region | (8) | | |
| | | CTAGAAACT-AT | bp MH | | | | |
| | | GGGGCAAACTGT | | | | | |
| | | CACCACTCCTTTG | | | | | |
| | | TCCC-3′ | | | | | |
| | | (SEQ ID NO: 69) | | | | | |
| | Junction | 5′-CTGTCATTTG | | | | | |
| | | TGCCCTCCTAAGC | | | | | |
| | | CTAGAAACTTATG | | | | | |
| | | GGGCAAACTGTC | | | | | |
| | | ACCACTCCTTTGT | | | | | |
| | | CCC-3′ | | | | | |
| | | (SEQ ID NO: 70) | | | | | |
| 4_9 | Genomic | 5′-CTGTCATTTG | 12 bp | Translation | MMEJ | 329.4 | 13.4% |
| | | TGCCCTCCTAAGC | deletion, 5 | inhibition | | | |
| | | CTAGAAACTATG | bp MH | | | | |
| | | GGGCAAACTGTC | | | | | |
| | | ACCACTCCTTTGT | | | | | |
| | | CCC-3′ | | | | | |
| | | (SEQ ID NO: 69) | | | | | |
| | Junction | 5′-CTGTCATTTG | | | | | |
| | | TGCCCTCCTAAGC | | | | | |
| | | CTAG--------- | | | | | |
| | | ---AAACTGTCAC | | | | | |
| | | CACTCCTTTGTCC | | | | | |
| | | C-3′ | | | | | |
| | | (SEQ ID NO: 71) | | | | | |

PPYP6 sgRNA (n = 4)

| | | | | | | | |
|---|---|---|---|---|---|---|---|
| 1_6 | Genomic | 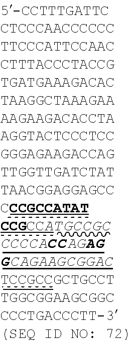 | 1: 27 bp deletion, 7 bp MH (89 bp upstream) 2: Deletion + Insertion (Replacement of 29 bp with 2 bp; net = 27 bp deletion), 15 bp MH | In-frame mutation | 1: SD- MMEJ (loop- out) 2: SD- MMEJ (loop- out) | — | — |
| | Junction | 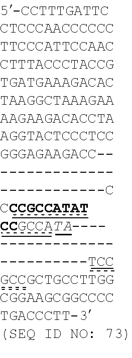 | | | | | |
| 3_1 | Genomic | 5′-GCCCCCGC | 1 bp | Frameshift | C-NHEJ | — | 3.1% |
| | | CATATCCGCC | insertion, 1 | mutation | | | |
| | | ACTGCCGCCCCCA | bp MH | | | | |
| | | C-CAGAGGCAG | | | | | |
| | | AAGCGGACTCCGC | | | | | |
| | | CGCTGCCTTG-3′- | | | | | |
| | | (SEQ ID NO: 74) | | | | | |
| | Junction | 5′-GCCCCCGC | | | | | |
| | | CATATCCGCC | | | | | |
| | | ACTGCCGCCCCCA | | | | | |
| | | CCCAGAGGCAGA | | | | | |
| | | AGCGGACTCCGCC | | | | | |
| | | GCTGCCTTG-3′ | | | | | |
| | | (SEQ ID NO: 75) | | | | | |
| 3_10 | Genomic | 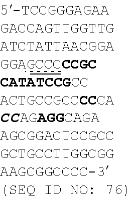 | 4 bp deletion, 2 bp MH or 5 bp MH (22 bp upstream) | Frameshift mutation | MMEJ or SD- MMEJ (loop- out) | NA | 1.2% |
| | Junction | 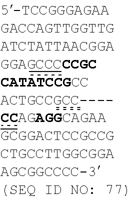 | | | | | |
| 8_6 | Genomic | 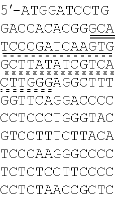 | Deletion + Insertion; (Replace-ment of 9 bp with 26 bp; net = 17 bp insertion); templated inversed insertion possible from same or other ERV allele (9) | Frameshift mutation | HR | — | — |
| | | 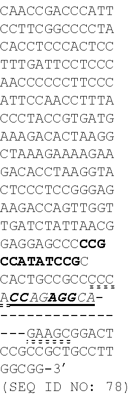 | | | | | |
| | Junction | 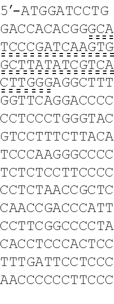 | | | | | |
| | | 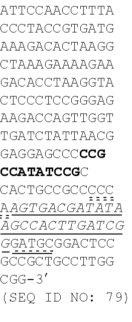 | | | | | |

PPYP13 sgRNA (n = 1)

| | | | | | | | |
|---|---|---|---|---|---|---|---|
| (5_8) | Genomic | 5′-TTAACGGAGG | 12 bp | In-frame | HR | — | — |
| | | AGCCCCCGCC | insertion | mutation | | | |
| | | ATATCCGCCAC | (10); likely | | | | |
| | | TGCCGCCCCCACC | from | | | | |
| | | AGAGGCAGAAGCG | another | | | | |
| | | GACTCCGCCGCTG | ERV allele | | | | |
| | | CCTTGGCGGAAGC | | | | | |
| | | GGCCCC------- | | | | | |
| | | -----TGACCCTT | | | | | |
| | | CACCAATGGCTTA-3′ | | | | | |
| | | (SEQ ID NO: 80) | | | | | |
| | Junction | 5′-TTAACGGAGG | | | | | |
| | | AGCCCCCGCC | | | | | |
| | | ATATCCGCCAC | | | | | |
| | | TGCCGCCCCCACC | | | | | |
| | | AGAGGCAGAAGCG | | | | | |
| | | GACTCCGCCGCTG | | | | | |
| | | CCTTGGCGGAAGC | | | | | |
| | | GGCCCCAGATCCA | | | | | |
| | | CCACCTGACCCTT | | | | | |
| | | CACCAATGGCTTA-3′ | | | | | |
| | | (SEQ ID NO: 81) | | | | | |

TABLE 5 shows mRNA Sanger sequencing data of the expressed ERV repair junctions of CHO-K1 cells treated with wild-type Cas9 nuclease and various sgRNAs (Myr2, PPYP6 and PPYP13). In column 2, the predicted blunt-ended DSB sites induced by the various sgRNAs and the wild-type Cas9 nuclease are highlighted in italicized Arial Black font (e.g., A). PAM site are shown in bolded Arial front (e.g., A). Myr and PPYP target motifs are highlighted in regular Arial Black font (e.g., A). Pre-existing microhomologies (MH) of the microhomology-mediated end-joining (MMEJ) repair mechanism are shown in bold, while de novo MH of the synthesis-dependent microhomology-mediated end-joining (SD-MMEJ) mechanism being underlined with a double line. Inserted bases are represented in bold letters, deleted bases with a “−” sign, and replacements in bold black. (8) Frequent 1 bp insertions consisting of a duplication of the 4th nucleotide were also observed previously (Lemos 2018, Taheri 2018), (9) DNA template sequence for insertion located 290 bp upstream, (10) DNA template sequence for insertion located 71 bp downstream.

Table 5, column 2 shows the predicted blunt-ended DSB sites induced by the various sgRNAs and the wild-type Cas9 nuclease are highlighted (see table legend for further details). In column 3, the size of mutation and MH length (in bp) is provided. The distance between priming site and the break site for de novo MH are shown in parenthesis. Column 4 shows that ERV mutation types include in-frame mutations, out-of-frame mutations, translation inhibition (mutation of the ATG translation initiation codon) or mutations locating outside of the ERV coding region. Out-of-frame mutations and translation inhibition are likely, while in-frame mutations and mutations outside of the coding region are less likely to influence ERV expression and VLP formation. In column 5, the most probable DSB repair mechanism based on manual junction analysis is indicated. Possible repair mechanisms include C-NHEJ, MMEJ, SD-MMEJ (snap-back), SD-MMEJ (loop-out), single strand annealing (SSA), homologous recombination (HR), and unknown. For snap-back SD-MMEJ mechanism, de novo priming sites are inverted repeats, while loop-out SD-MMEJ mechanisms uses priming sites with direct repeats (Khodaveridan 2017). If the observed junction sequence is compatible with more than one mechanism and both appear equally likely, all potential pathways are listed. Junctions were verified for homologies at break site and templated insertions (SD-MMEJ) using program described in Schimmel et al. 2017 (Schimmel 2017). Colum 6 shows the score of each repair pattern according to the MH size and the deletion length. Pattern score was calculated using the RGenome “Microhomology-Predictor” tool (on the rgenome.net website under mich-calculator) described in Bae et al. 2014 (Bae 2014). The higher the score, the more likely the predicted mutation should be observed. The pattern score is only valid for repair junctions showing MHs at the break site (MMEJ-mediated repair). Column 7 shows the predicted frequencies of CRISPR-Cas9 editing outcomes using the online tool FORECasT1® (Favored Outcomes of Repair Events at Cas9 Targets: o the partslab.sanger.ac.uk website under FORECasT®) as described in Allan et al. 2018 (Allan 2018). The higher the frequency. the more junctions are expected to contain the predicted mutation pattern. Only the frequencies of the predicted ten most frequent mutations are listed.

### Isolation and Characterization of ERV-Mutated CHO-K1 Clones

Given that roughly 10-15% of the expressed group 1 ERV sequences are predicted to be mutated, it was hypothesized that a potential reduction in viral particle release would be difficult to detect within a polyclonal population. Thus, single CHO cell clones were isolated from bulk-sorted Myr2- or PPYP6-edited cell pools, and screened for those having mutations in the expressed group 1 ERV sequences. 18 out of 95 (18%) and 14 out of 181 (8%) Myr2 and PPYP6 sgRNA-treated clones, respectively, contained group 1 ERV mutations at the mRNA level, in line with previous estimations (TABLE 6, also TABLE 4, 5).

TABLE 6
Detection of CRISPR-mediated mutations in the expressed
type-C ERV sequences of edited CHO-K1 clones.

| | # screened | # mutated | Mutation | Loss-of-function |
|---|---|---|---|---|
| Sample | clones | clones | frequency | mutation frequency* |
| Myr2 sgRNA | 95 | 18 | 19% | 11% |
| PPYP6 sgRNA | 181 | 14 | 8% | 79% |
| Total | 276 | 32 | 12% | 45% |

*Includes translation inhibition and frameshift mutations and is expressed relative to the number of mutated clones.

Among the Myr2-mutated clones, the majority possessed an identical 1 bp insertion upstream of the ATG start codon (TABLE 7), which likely resulted from staggered CRISPR-Cas9 cleavage (39). No clone treated with the PPYP6 sgRNA acquired a mutation spanning the PPYP motif. Nonetheless, two Myr2- and eleven PPYP6-derived clones contained mutations either blocking translation or frameshifting the gag transcripts, hence making them promising candidates for reduced viral particle release. It was also observed that the Sanger sequencing chromatogram of the repair junctions of all clones showed a clear singly mutated sequence and lacked background noise in the CRISPR flanking sequences. This supported the hypothesis that only a single group 1 ERV locus might be prominently transcribed and leads to the production of viral particles by CHO cells.

TABLE 7
Sequence analysis of the expressed mRNA ERV sequences of mutated CHO-K1
clones.

| | | | | Repair | Pattern | FORE |
|---|---|---|---|---|---|---|
| | | | Mutation | pathway | score | CasT |
| Clone (1) | Sequence (2) | Features (3) | type (4) | (5) | (6) | (7) |

Myr2 sgRNA (n = 18)

| | | | | | | | |
|---|---|---|---|---|---|---|---|
| C02 | Genomic | 5′-TGTCATTTGTGCCCTCCTAA | 2 bp deletion, | Translation | C-NHEJ | — | 2.6% |
| | | GCCTAGAAACTATGGGGCAA | 1 bp MH | inhibition | | | |
| | | ACTGTCACCACTCCTTTGTCC | | | | | |
| | | CTAACACTCTCCCACTGGAA-3′ | | | | | |
| | | (SEQ ID NO: 82) | | | | | |
| | Junction | 5′-TGTCATTTGTGCCCTCCTAA | | | | | |
| | | GCCTAGAAAC-TGGGGCAAA | | | | | |
| | | CTGTCACCACTCCTTTGTCCC | | | | | |
| | | TAACACTCTCCCACTGGAA-3′ | | | | | |
| | | (SEQ ID NO: 83) | | | | | |
| D12 | Genomic | 5′-CGACTCTCTCTCAATTCCT- | 114 bp | Translation | MMEJ | NA | — |
| | | 75bp-GAAACTATGGGGCAAAC | deletion, 3 bp | inhibition | | | |
| | | TGTCACCACTCCTTTGT-3′ | MH | | | | |
| | | (SEQ ID NO: 84) | | | | | |
| | Junction | 5′-CGACTCTCTCTCAA---95b | | | | | |
| | | p---=-----ACTGTCACCACT | | | | | |
| | | CCTTTGT-3′ | | | | | |
| | | (SEQ ID NO: 85) | | | | | |
| G09 | Genomic | 5′-TCTTTGTCTTGTAGCTGTCA | 27 bp | Outside | MMEJ | 51.8 | — |
| | | TTTGTGCCCTCCTAAGCCTAG | deletion, 2 bp | ERV | | | |
| | | AAACTATGGGGCAAACTGTCA | MH | coding | | | |
| | | CCACTCCTTTGTCCCTAACAC | | region | | | |
| | | TCTCCCACTGGAAAGATGTAC | | | | | |
| | | AGGAATATGCTCATAACCAAT | | | | | |
| | | CT-3′ | | | | | |
| | | (SEQ ID NO: 86) | | | | | |
| | Junction | 5′-TCTTTGTCTTGTAGCTGTC-- | | | | | |
| | | --------------ATGGGGCA | | | | | |
| | | AACTGTCACCACTCCTTTGTC | | | | | |
| | | CCTAACACTCTCCCACTGGAA | | | | | |
| | | AGATGTACAGGAATATGCTCA | | | | | |
| | | TAACCAATCT-3′ | | | | | |
| | | (SEQ ID NO: 87) | | | | | |
| H02 | Genomic | 5′-AGCTGTCATTTGTGCCCTC | 3 bp deletion, | Outside | C-NHEJ | — | — |
| | | CTAAGCCTAGAAACTATGGGG | no MH | ERV | | | |
| | | CAAACTGTCACCACTCCTTTG | | coding | | | |
| | | TCCC-3′ | | region | | | |
| | | (SEQ ID NO: 88) | | | | | |
| | Junction | 5′-AGCTGTCATTTGTGCCCTC | | | | | |
| | | CTAAGCCTAGA--TATGGGGC | | | | | |
| | | AAACTGTCACCACTCCTTTGT | | | | | |
| | | CCC-3′ | | | | | |
| | | (SEQ ID NO: 89) | | | | | |
| A04 | Genomic | 5′-CTCCTAAGCCTAGAAACT-A | 1 bp insertion, | Outside | C-NHEJ | — | 17.8% |
| (n = 14) | | TGGGGCAAACTGTCACCACT | 1 bp MH | ERV | (8) | | |
| | | CC-3′ | | coding | | | |
| | | (SEQ ID NO: 90) | | region | | | |
| | Junction | 5′-CTCCTAAGCCTAGAAACTT | | | | | |
| | | ATGGGGCAAACTGTCACCACT | | | | | |
| | | CC-3′ | | | | | |
| | | (SEQ ID NO: 91) | | | | | |

PPYP6 sgRNA (n = 14)

| | | | | | | | |
|---|---|---|---|---|---|---|---|
| A02 | Genomic | 5′-CCCCCGCCATATCCGCCAC | Deletion + | Frameshift | HR | — | — |
| | | TGCCGCCCCCACCAGAGGCA | Insertion | mutation | | | |
| | | GAAGCGGACTCCGCCGCTGC | (Replace-ment | | | | |
| | | CTTGGCGGAAGC-3′ | of 20 bp with | | | | |
| | | (SEQ ID NO: 92) | 10 bp; net = | | | | |
| | | | 10 bp deletion); | | | | |
| | | | inverted | | | | |
| | | | templated | | | | |
| | | | insertion from | | | | |
| | | | three possible | | | | |
| | | | ERV alleles | | | | |
| | Junction | 5′-CCCCCGCCATATCCGCCAC | | | | | |
| | | TGCCGCCCCACTGCTTCTG--- | | | | | |
| | | -----TCCGCCGCTGCCTTGGC | | | | | |
| | | GGAAGC-3′ | | | | | |
| | | (SEQ ID NO: 93) | | | | | |
| A07 | Genomic | 5′-CCCCCGCCATATCCGCCAC | 7 bp deletion, | Frameshift | Unknown | — | 1.1% |
| | | TGCCGCCCCCACCAGAGGCA | 2 bp MH | mutation | (9) | | |
| | | GAAGCGGACTCCGCCGCTGC | flanking DSB | | | | |
| | | CTTGGC-3′ | | | | | |
| | | (SEQ ID NO: 94) | | | | | |
| | Junction | 5′-CCCCCGCCATATCCGCCAC | | | | | |
| | | TGCCGCCCCCA----CAGAAG | | | | | |
| | | CGGACTCCGCCGCTGCCTTG | | | | | |
| | | GC-3′ | | | | | |
| | | (SEQ ID NO: 95) | | | | | |
| B11 | Genomic | 5′-CCCCCGCCATATCCGCCAC | 1 bp insertion, | Frameshift | C-NHEJ | — | 3.1% |
| (n = 3) | | TGCCGCCCCCAC-CAGAGGC | 1 bp MH | mutation | | | |
| | | AGAAGCGGACTCCGCCGCTG | | | | | |
| | | CCTTGGC-3′ | | | | | |
| | | (SEQ ID NO: 94) | | | | | |
| | Junction | 5′-CCCCCGCCATATCCGCCAC | | | | | |
| | | TGCCGCCCCCACCCAGAGGC | | | | | |
| | | AGAAGCGGACTCCGCCGCTG | | | | | |
| | | CCTTGGC-3′ | | | | | |
| | | (SEQ ID NO: 96) | | | | | |
| D08 | Genomic | 5′-GGAGAAGACCAGTTGGTTG | 9 bp deletion, | In-frame | MMEJ | 191.4 | 2% |
| | | ATCTATTAACGGAGGAGCCCC | 2 bp MH | mutation | | | |
| | | CCGCCATATCCGCCACTGCC | | | | | |
| | | GCCCCCACCAGAGGCAGAAG | | | | | |
| | | CGGACTCCGCC-3′ | | | | | |
| | | (SEQ ID NO: 97) | | | | | |
| | Junction | 5′-GGAGAAGACCAGTTGGTTG | | | | | |
| | | ATCTATTAACGGAGGAGCCCC | | | | | |
| | | CCGCCATATCCGCCACTGCC | | | | | |
| | | GCCCC------CAGAAGCGGAC | | | | | |
| | | TCCGCC-3′ | | | | | |
| | | (SEQ ID NO: 98) | | | | | |
| E10 | Genomic | 5′-AGCCCCCGCCATATCCGCC | 37 bp | Frameshift | MMEJ | 172.7 | — |
| | | ACTGCCGCCCCCACCAGAGG | deletion, 6 bp | mutation | | | |
| | | CAGAAGCGGACTCCGCCGCT | MH | | | | |
| | | GCCTTG-3′ | | | | | |
| | | (SEQ ID NO: 99) | | | | | |
| | Junction | 5′-AGCCCCCGCCATATCCGCC | | | | | |
| | | ------------------GCT | | | | | |
| | | GCCTTG-3′ | | | | | |
| | | (SEQ ID NO: 100) | | | | | |
| G12 | Genomic | 5′-CCCCCGCCATATCCGCCAC | 3 bp deletion, | In-frame | MMEJ or | 258.3 | 14.8% |
| (n = 2) | | TGCCGCCCCCACCAGAGGCA | 3 bp MH or 4 | mutation | SD-MMEJ | | |
| | | GAAGCGGACTCCGCCGCTGC | bp MH (4 bp | | (loop-out) | | |
| | | CTTGGC-3′ | downstream) | | | | |
| | | (SEQ ID NO: 101) | | | | | |
| | Junction | 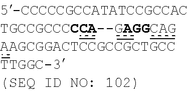 | | | | | |
| K3 | Genomic | 5′-CCCCCGCCATATCCGCCAC | 22 bp | Frameshift | MMEJ or | 133.1 | — |
| | | TGCCGCCCCCACCAGAGGCA | deletion, 2 bp | mutation | SD-MMEJ | | |
| | | GAAGCGGACTCCGCCGCTGC | MH or 5 bp | | (snap-back) | | |
| | | CTTGGCGGAAGCGG-3′ | MH (6 bp | | | | |
| | | (SEQ ID NO: 103) | upstream) | | | | |
| | Junction | 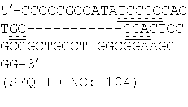 | | | | | |
| K9 | Genomic | 5′-CCCCCGCCATATCCGCCAC | 1 bp deletion, | Frameshift | C-NHEJ | — | 19.5% |
| (n = 2) | | TGCCGCCCCCAACCAGAGGCA | 1 bp MH | mutation | | | |
| | | GAAGCGGACTCCGCCGCTGC | | | | | |
| | | C-3′ | | | | | |
| | | (SEQ ID NO: 105) | | | | | |
| | Junction | 5′-CCCCCGCCATATCCGCCAC | | | | | |
| | | TGCCGCCCCCAC-AGAGGCA | | | | | |
| | | GAAGCGGACTCCGCCGCTGC | | | | | |
| | | C-3′ | | | | | |
| | | (SEQ ID NO: 106) | | | | | |
| K12 | Genomic | 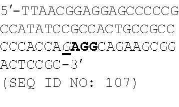 | Deletion + Insertion (Replace-ment of 3 bp with 1 bp; net = 2 bp deletion) | Frameshift mutation | Unknown | — | — |
| | Junction | 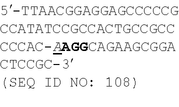 | | | | | |
| K14 | Genomic | 5′-AGGAGCCCCCGCCATATCC | 13 bp | Frameshift | MMEJ or | 208.8 | — |
| | | GCCACTGCCGCCCCCACCAG | deletion, 2 bp | mutation | SD-MMEJ | | |
| | | AGGCAGAAGCGGACTCCCCC | MH or 5 bp | | (loop-out) | | |
| | | GCCATATCCGCGGAAGCGGC | MH (15 bp | | | | |
| | | CCCTGACCCTT-3′ | downstream) | | | | |
| | | (SEQ ID NO: 109) | | | | | |
| | Junction | 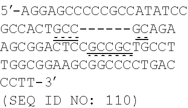 | | | | | |

Table 7 shows mRNA Sanger sequencing data of the expressed ERV repair junctions of CHO-K1 clones treated with wild-type Cas9 nuclease and the Myr2 or PPYP6 sgRNAs (Junctions). versus the unmutated sequence of the parental non-engineered Cho cell (Genomic). The sequences are derived from Sanger sequencing of cDNA PCR amplicons. If the same repair junction was detected more than once, the number is indicated below each sample name as (n=). In column 2, predicted blunt-ended DSB sites induced by the two sgRNAs and the wild-type Cas9 nuclease are highlighted in italicized Arial Black font (e.g., A), PAM sites and Myr and PPYP target motifs are highlighted in regular Arial Black font (e.g., A). Pre-existing microhomologies (MH) of the microhomology-mediated end-joining (MMEJ) repair mechanism are shown in bold grey letters (e.g. GC), while de novo MH of the synthesis-dependent microhomology-mediated end-joining (SD-MMEJ) mechanism are underlined with a double line. Inserted bases are represented in small bold Courier letters (e.g., c), deleted bases with a “−” sign, and replacements in italic underlined with a single bold line. (the dark highlighted boxes contain GGG). NA: not available. (8) Frequent i bp insertions consisting of a duplication of the 4th nucleotide were also observed previously (Lemos2018, Taheri2018). (9) Unknown mechanism but similar junction pattern was described in Shin et al. 2017 (Shin 2017).

To further investigate the CRISPR-derived mutations at the genome level, the Myr and PPYP flanking regions of type-C ERVs were deep sequenced in a subset of CHO clones bearing mutations in the expressed ERV sequences (TABLE 7). Two Myr2- and four PPYP6-edited clones with Gag loss-of-function mutations were selected in the expressed group 1 type-C ERV sequences (clones CO2 and D12 for Myr2; A02, E10, K03 and K14 for PPYP6) as well as one Myr2-derived clone with a large mutation outside of the group 1 ERV coding (G09) and genotyped them along with wild-type and empty vector control samples.

Table 7 shows mRNA Sanger sequencing data of the expressed ERV repair junctions of CHO-K1 clones treated with wild-type Cas9 nuclease and the Myr2 or PPYP6 sgRNAs. The sequences are derived from Sanger sequencing of cDNA PCR amplicons. In column 2 the predicted blunt-ended DSB sites induced by the two sgRNAs and the wild-type Cas9 nuclease are highlighted (see table legend for further details). In column 3, the size of mutation and MH length (in bp) is provided. The distance between priming site and the break site for de novo MH are shown in parenthesis.

Column 4 indicates that ERV mutation types include in-frame mutations, out-of-frame mutations, translation inhibition (mutation of the ATG translation initiation codon) or mutations locating outside of the ERV coding region. Out-of-frame mutations and translation inhibition are likely, while in-frame mutations and mutations outside of the coding region are less likely to influence ERV expression and VLP formation. In column 5, the most probable DSB repair mechanism based on manual junction analysis. Possible repair mechanisms include C-NHEJ, MMEJ, SD-MMEJ (snap-back). SD-MMEJ (loop-out), single strand annealing (SSA), homologous recombination (HR), and unknown. For snap-back SD-MMEJ mechanism. de novo priming sites are inverted repeats, while loop-out SD-MMEJ mechanisms uses priming sites with direct repeats (Khodaveridan 2017). If the observed junction sequence is compatible with more than one mechanism and both appear equally likely, all potential pathways are listed. Junctions were verified for homologies at break site and templated insertions (SD-MMEJ) using program described in Schimmel et al. 2017 (Schimmel 2017). Column 6 shows the score of each repair pattern according to the MH size and the deletion length. Pattern score was calculated using the RGenome “Microhomology-Predictor” tool (http://www.rgenome.net/mich-calculator/) described in Bae et al. 2014 (Bae2014). The higher the score, the more likely the predicted mutation should be observed. The pattern score is only valid for repair junctions having MHs at the break site (MMEJ-mediated repair). Column 7, shows the predicted frequencies of CRISPR-Cas9 editing outcomes using the online tool FORECasT (Favoured Outcomes of Repair Events at Cas9 Targets; https://partslab.sanger.ac.uk/FORECasT) as described in Allan et al. 2018 (Allan 2018). The higher the frequency, the more junctions are expected to contain the predicted mutation pattern. Only the frequencies of the predicted ten most frequent mutations are listed.

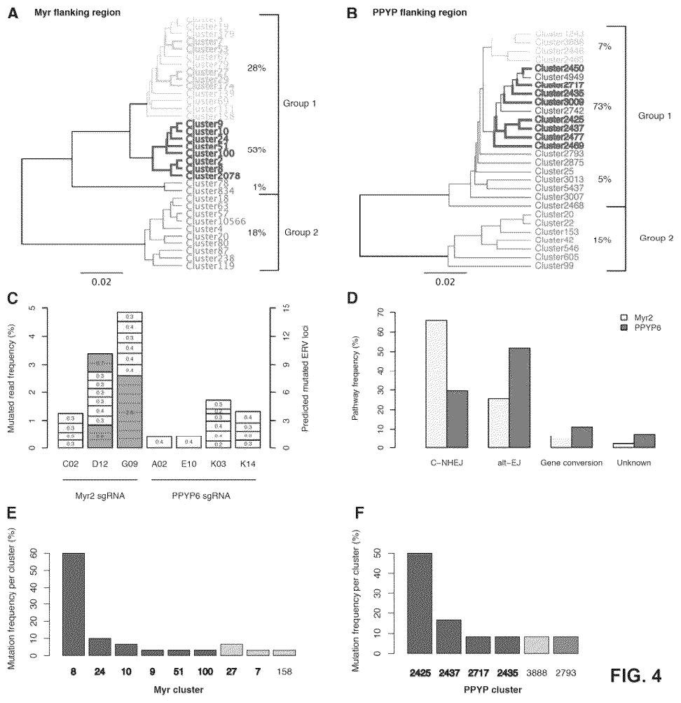

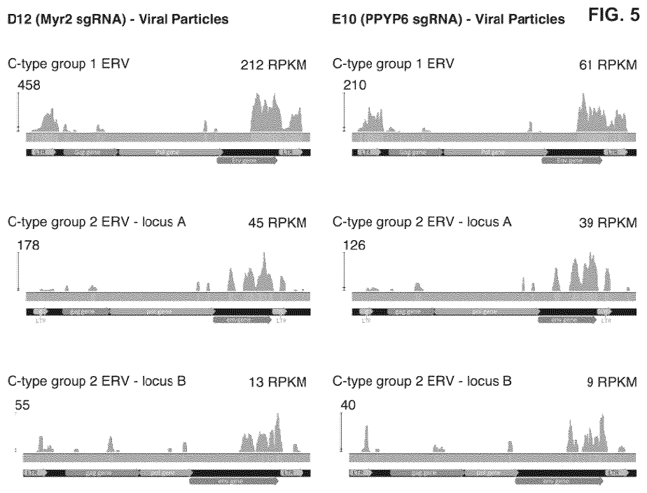

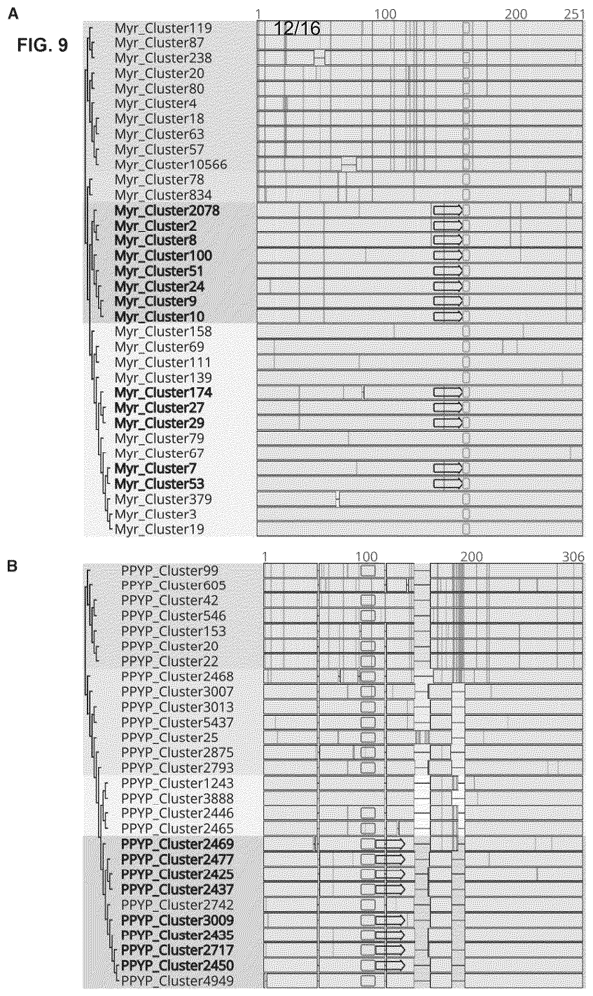

To detect CRISPR-derived mutations and distinguish them from sequence variations naturally occurring at each target, the reads from wild-type CHO cells were clustered and these cluster consensus sequences were used to create diversity profiles. When clustering by 97% sequence similarity, 34 Myr and 28 PPYP clusters were identified that represented the natural ERV sequence diversity present within the Myr and PPYP flanking regions (FIGS. 4A and 4B, FIG. 9). Despite the overall high sequence diversity, the Myr and PPYP motifs themselves were highly conserved, in agreement with their biological significance for viral budding. The identified clusters correlated well with the type-C ERV groups previously characterized from the CHO genome as well as with their predicted frequencies, corroborating the characterization of ERV sequences at the whole genome level (FIG. 1, FIGS. 4A and 4B).

For both targets, the largest cluster encompassed approximately 40% of all reads, and it was at least four-fold more abundant than the second largest cluster (highlight, FIGS. 4A and 4B). Interestingly, the consensus sequence of the largest clusters also conformed to the group 1 type-C ERV sequence determined from CHO viral particles. Among all clusters, 13 Myr and 8 PPYP clusters could be targeted by the Myr2 and PPYP6 sgRNAs, accommodating for 61% and 72% of the captured read diversity, respectively (bold letters, FIGS. 4A and 4B).

Using these wild-type CHO clusters and diversity profile, between 1 and 7 distinct CRISPR-derived mutations per clone were found, including the mutations already detected at the mRNA level (number of boxes. FIG. 4C). The detected mutation range spanned from a 114 bp deletion up to a 78 bp insertion. As expected, CHO cells treated with the empty vector expression plasmid lacked additional mutations in the CRISPR target sites. Some mutations, for instance a i bp insertion, occurred within all three genotyped Myr2-treated clones but were absent in the PPYP6 clones, as expected from sgRNA-specific repair outcomes (40).

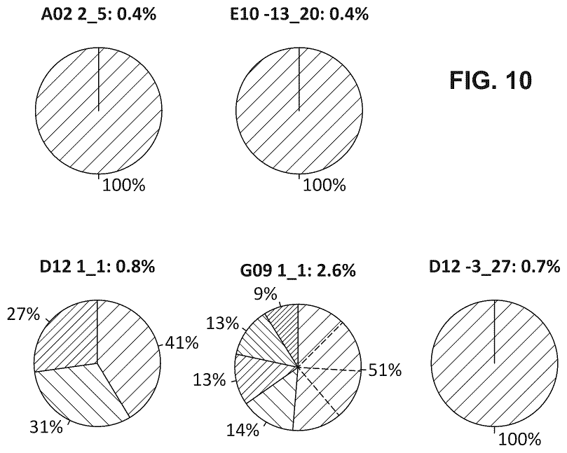

Typically, a given mutation was detected at a read frequency of approximately 0.3%, which thus must represent a single ERV locus in the CHO genome (FIG. 4C). However, three Myr2-derived mutations were discovered at a read frequency well above 0.3%, with the same 1 bp insertion being present in 2.6% of all G09 clone reads. Consequently, this implies that the same mutation may occur more than once in the same clone. In support for this hypothesis, the reads of predicted single locus mutations (i.e. clones A02 or E10) were highly similar in the mutation flanking region. while the reads of abundant mutations (i.e. G09 1_1) contained variations in the mutation flanking regions, suggesting that the same mutations may have occurred repeatedly at distinct ERV loci (FIG. 10). In the case of G09 1_1, five ERV groups could be distinguished with one group having four-times more reads than the others, indicating that this mutation should have occurred at eight distinct ERV loci in the G09 clone. Therefore, it was concluded that each clone acquired between 1 and 14 ERV mutations following transient CRISPR transfection (FIG. 4C). The identification of clones having only one mutated ERV at the DNA level, together with the finding that this mutation was identical to the single mutation detected at the intracellular RNA level, further substantiated that a single group 1 type-C ERV locus is transcribed, and likely responsible for the release of type-C retroviral particles from CHO cells.

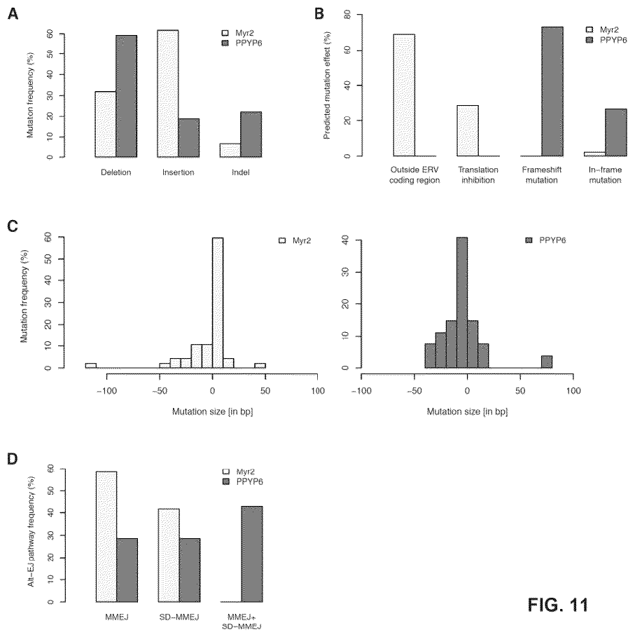

The repeated occurrence of identical mutations within one clone raised the question of whether they may result from gene conversion, an homologous recombination (HR)-related repair mechanism, in which a previous mutated ERV locus is used as template to repair other cleaved ERV sites. To find evidence for HR activity following Myr2- and PPYP6-mediated cleavage, the previously obtained mRNA and DNA data were combined and a total of 74 DNA repair junctions (n_{Myc}=47, n_{PPYP}=27) were analyzed. While Myr2 sgRNA-mediated cleavage led to an overall higher mutation frequency, with a preference for insertions, PPYP6 sgRNA mostly produced deletions. Notably, Gag loss-of-function mutations were observed in 70% of PPYP6 sgRNA-induced repaired junctions, but only in 30% of all Myr2 sgRNA-derived mutations (FIG. 11B). The majority of Myr2- and PPYP6-derived repair junctions were compatible with classical non-homologous end-joining (C-NHEJ) and alternative end-joining (alt-EJ) repair activities (FIG. 4D). C-NHEJ typically leads to small insertion and deletions, while aft-EJ utilizes microhomologies at the DSB site to anneal broken ends, which often results in larger and more complex mutations. Although alt-EJ repair is considered to be a backup pathway in most mammalian cells, between 25%-55% aft-EJ compatible junctions were detected when targeting the gag gene, supporting conclusions of intrinsically elevated alt-EJ activities in CHO cells (41, 42). Among the alt-EJ repair junctions, some could be uniquely attributed to the microhomology-mediated end-joining (MMEJ) or the synthesis-dependent microhomology-mediated end-joining (SD-MMEJ) aft-EJ subpathways, while others were consistent with both MMEJ and SD-MMEJ repair (43, 44) (FIG. 11D). Interestingly, approximately 10% of all analyzed repair junctions contained either insertions templated from other ERV loci or from the same ERV locus but using a distant sequence, while others manifested apparent duplications devoid of microhomologies, as mediated by alt-EJ mechanisms. All of these latter junctions are consistent with homology-directed repair activities at Myr2- and PPYP6 target sites following CRISPR cleavage (FIG. 4D). Thus, HR-mediated gene conversion might indeed have caused the multiple occurrences of certain mutations.

Next, it was assessed whether mutations occurred more frequently in some type-C ERV clusters, indicating a preferential cleavage of certain ERV loci. As expected, mutations associated uniquely with clusters of group 1, but not of group 2, confirming sgRNA specificity for group 1 only (FIGS. 4E and 4F). The majority of mutations located within the most abundant Myr or PPYP clusters, which represent the actively transcribed and hence expressed ERVs. The other mutations were seen in additional clusters, although at lower frequencies, all of which contained a Myr2 or PPYP6 sgRNA recognition sites adjacent to a PAM sequence (FIGS. 4E and 4F, bold font). Surprisingly, CRISPR cleavage in Myr and PPYP clusters containing a one base pair mismatch to the sgRNA target site were also observed, supporting previous reports indicating that CRISPR-Cas9 tolerates small mismatches during target recognition (45) (FIGS. 4E and 4F, normal font). Overall, it was concluded that the clusters associated with a high frequency of mutations most likely encompass expressed ERV loci.

### Identification of a Unique Viral Particle (VP)-Producing ERV Locus in CHO-K1 Cells

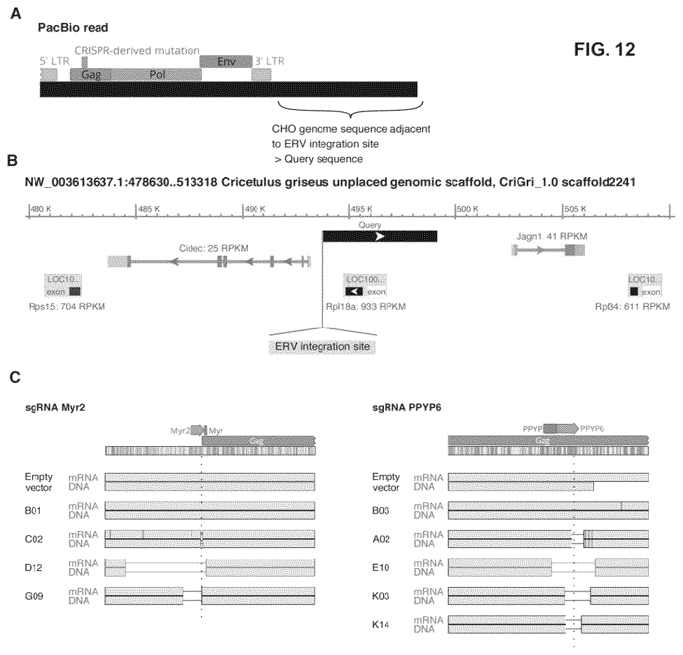

The Sanger chromatograms as well as the read frequencies of gag mutations observed during RNA and targeted DNA amplicon sequencing, respectively, corroborated the assumption that a single group 1 type-C ERV locus is transcribed, and may therefore mediate viral particle production by CHO cells. To further substantiate this assumption, the genome of the E10 clone was fully sequenced using the PacBio® approach, so as to obtain reads sufficiently long for the unambiguous determination of ERV-containing loci. This clone was selected as it appeared to contain only a single mutated ERV, so as to correlate its unique mutation at the RNA level with a potentially unique genomic locus (FIG. 4C). Analysis of the E10 clone genome sequence led to the identification of a single ERV locus bearing the mutation detected at the mRNA level (FIGS. 12A and 12B). The predicted ERV integration site was then validated by PCR amplification and DNA Sanger sequencing using locus-specific primers located outside of the ERV sequence in the parental CHO cell line as well as the deep-sequenced clones. All deep-sequenced clones, which contain CRISPR-derived mutations at the mRNA level, possessed the identical mutation also at this ERV locus, further supporting that this genomic region harbors the expressed type-C ERV element (FIG. 12C). Interestingly, this particular ERV integration was found to be hemizygous, as the other allele was devoid of a corresponding ERV integration, and to have occurred into open chromatin between two moderately expressed CHO cell genes.

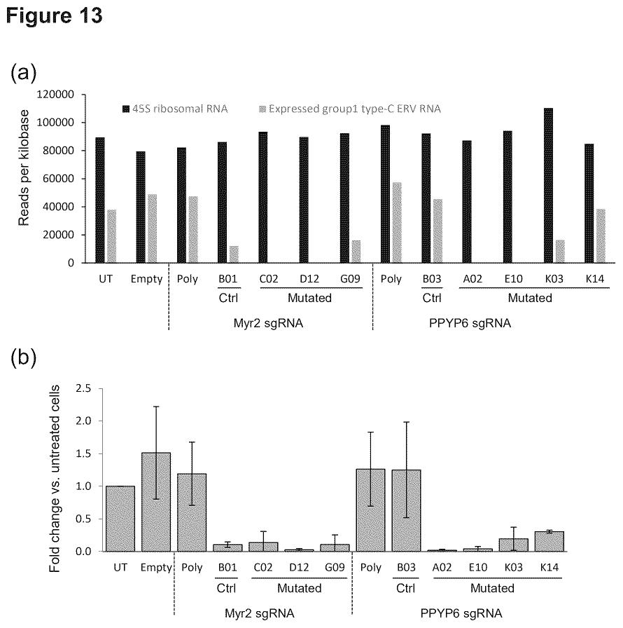

Next, it was assessed whether Gag loss-of-function mutations in this expressed ERV locus may lead to the anticipated inhibition of viral particle budding. Besides the previously characterized mutated clones, we analyzed in parallel their corresponding bulk-sorted polyclonal populations, as well as a clone devoid of detectable mutations in the expressed group 1 ERV sequence (B01 for Myr2, B03 for PPYP6), as additional controls. First, viral particles were extracted from the supernatant of the CHO cell cultures and the amount of type-C viral genomes was quantified by RT-qPCR. Preliminary data suggested that viral particles shed by Gag loss-of-function mutants contain 80% less group 1 C-type genomic viral RNA than control samples. while the amount of group 2 genomic viral RNA remained close to detection limit (data not shown). To substantiate this finding, RNA extracted from the viral particles shed by the D12 (Myr2 sgRNA) and E10 (PPYP6 sgRNA) clones was Illumina deep-sequenced. Remarkably, a more than 250-fold reduction in reads mapping to the group 1 ERV sequence in both D12 and E10 were observed when compared to wild-type CHO cells, while the trace amounts of reads mapping to group 2 remained close to the detection level (compare FIG. 13). This indicated that mutations in the single expressed group 1 ERV sequence that block translation initiation (D12) or introduce a frameshift in the gag gene downstream of the PPYP motif (E10) are sufficient to severely reduce the budding of complete viral particles.

### Characterization of Edited CHO Cell Lines Displaying Reduced Viral Budding

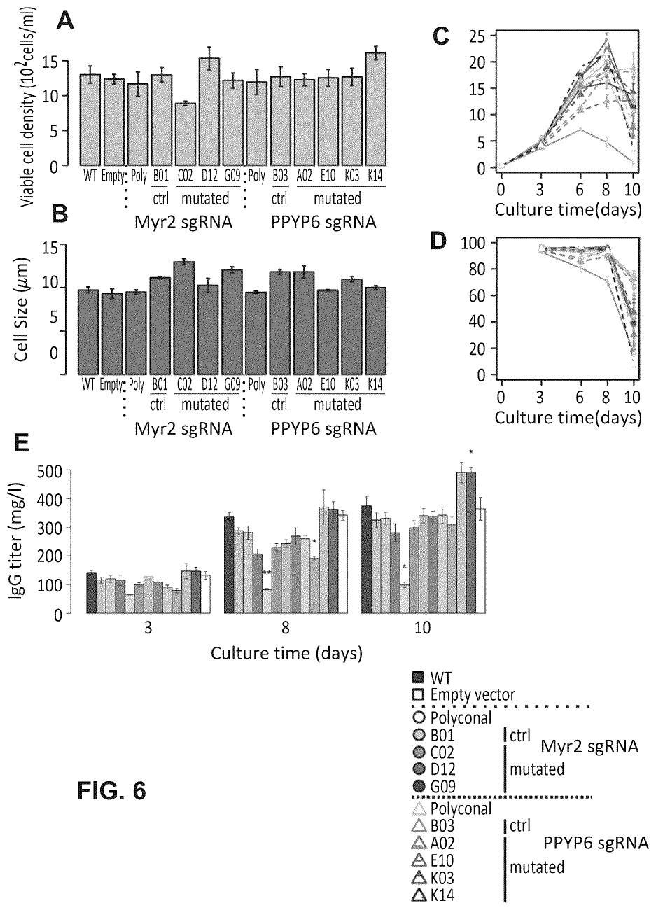

Having observed that CRISPR mutagenesis had efficiently inactivated viral particle release, it was next tested whether ERV inactivation would affect other CHO cell properties, such as cell growth, cell size and therapeutic protein production. ERV-edited clones were found to proliferate at similar rates as polyclonal populations, wild-type and empty vector-treated cell controls, with a density reaching approximately 12.5×10⁶ cells/ml after five days in culture (FIG. 6A). Such a cell density concords with the expected CHO-K1 doubling time of roughly 20h (46). Although two Myr2 sgRNA clones (C02, D12) and one PPYP6 sgRNA clones (K14) showed slightly modified cell cycle durations, the effect was not statistically significant. In addition, cell sizes tended to be elevated in ERV-edited cells, notably in the C02 clone, but they did not differ significantly when compared to the empty vector control cells (FIG. 6B).

Finally, the capacity of ERV-edited CHO cells to produce therapeutic proteins was assessed, a pivotal property of CHO cells for biotechnological use. The previously characterized ERV-mutated cells were used to generate polyclonal populations stably expressing a humanized therapeutic IgG immunoglobulin and quantified IgG secretion during ten-days fed-batch cultures. ERV-edited clones and polyclonal populations expressing the IgG protein demonstrated cell growth and cell viability properties similar to those of wild-type and empty vector control cells, as observed without therapeutic protein expression (FIGS. 6C and 6D). IgG titers in the cell culture supernatants increased over the course of the fed-batch experiment, as expected from the accumulation of the secreted IgG protein, reaching around 300-400 mg/I at the end of the fed-batch for control cells and most ERV-edited cell clones (FIG. 6E). Thus, ERV mutagenesis did not globally affect the capability of CHO cells to produce IgG proteins, although clone C02 (Myr2 sgRNA) secreted significantly less immunoglobulins, likely reflecting its reduced growth and increased cell size, while clones E10 and K03 (both PPYP6 sgRNA) produced 50% more IgG relative to the empty vector control. Overall, this indicated that CHO clones that were exposed to multi-loci ERV editing generally maintain normal CHO characteristics, while some clones, especially those with mutations in the PPYP region, appeared to have acquired a higher metabolic capacity to produce therapeutic proteins. However, this apparently augmented metabolism capacity could not be correlated to a specific ERV mutation type or to the total number of mutations, nor to cell growth or size, suggesting clone-specific effects.

As the person skilled in the art will appreciate, the above description is not limiting, but provides examples of certain embodiments of the present invention. With the guidance provided above, the person skilled in the art is able to devise a wide variety of alternatives not specifically set forth herein.

### REFERENCES

1. Berting, A., Farcet, M. R. and Kreil, T. R. (2010) Virus susceptibility of Chinese hamster ovary (CHO) cells and detection of viral contaminations by adventitious agent testing. Biotechnol. Bioeng., 106, 598-607.
2. Hartley, J. W. and Rowe, W. P. (1976) Naturally occurring murine leukemia viruses in wild mice: characterization of a new “amphotropic” class. J. Virol., 19, 19-25.
3. Shepherd, A. J., Wilson, N. J. and Smith, K. T. (2003) Characterisation of endogenous retrovirus in rodent cell lines used for production of biologicals. Biologicals, 31, 251-260.
4. Hojman, F., Emanoil-Raivier, R., Lesser, J. and Périès, J. (1989) Biological and molecular characterization of an endogenous retrovirus present in CHO/HBs-A Chinese hamster cell line. Dev. Biol. Stand., 70, 195-202.
5. Emanoil-Ravier, R., Hojman, F., Servenay, M., Lesser, J., Bernardi, A. and Peries, J. (1991) Biological and molecular studies of endogenous retrovirus-like genes in Chinese hamster cell lines. Dev. Biol. Stand., 75, 113-122.
6. Dinowitz, M., Lie, Y. S., Low, M. A., Lazar, R., Fautz, C., Potts, B., Sematinger, J. and Anderson, K. (1992) Recent studies on retrovirus-like particles in Chinese hamster ovary cells. Dev. Biol. Stand., 76, 201-207.
7. Manly, K. F., Givens, J. F., Taber, R. L. and Zeigel, R. F. (1978) Characterization of Virus-like Particles Released from the Hamster Cell Line CHO-K1 After Treatment with 5-Bromodeoxyuridine. J. Gen. Virol., 39, 505-517.
8. Gould, R. R. and Borisy, G. G. (1977) The pericentriolar material in Chinese hamster ovary cells nucleates microtubule formation. J. Cell Biol., 73, 601-615.
9. Heine U, Kramarsky B, Wendel E, S. R. (1979) Enhanced Proliferation of Endogenous Virus in Chinese Hamster Cells Associated with Microtubules and the Mitotic Apparatus of the Host Cell. J. Gen. Virol., 44, 45-55.
10. Anderson, K. K., Low, M.-A., Lie, Y. Y., Keller, G.-A. and Dinowitz, M. (1991) Endogenous origin of defective retroviruslike particles from a recombinant Chinese hamster ovary cell line. Virology, 181, 305-311.
11. Reuss, F. U. (1992) Expression of intracisternal A-particle-related retroviral element-encoded envelope proteins detected in cell lines. J. Virol., 66, 1915-1923.
12. Lie, Y. Y., Penuel, E. E., Low, M. M., Nguyen, T. T., Mangahas, J. J., Anderson, K. K. and Petropoulos, C. C. (1994) Chinese hamster ovary cells contain transcriptionally active full-length type C proviruses. J. Virol., 68, 7840-7849.
13. Anderson, K. K., Lie, Y. Y., Low, M. M., Williams, S. S., Fennie, E. E., Nguyen, T. T. and Wurm, F. F. (1990) Presence and transcription of intracisternal A-particle-related sequences in CHO cells. J. Virol., 64, 2021-2032.
14. Tihon, C. and Green, M. (1973) Cyclic AMP-amplified Replication of RNA Tumour Virus-like Particles in Chinese Hamster Ovary Cells. Nat. New Biol., 244, 227.
15. Wurm, F. (2013) CHO Quasispecies—Implications for Manufacturing Processes. Processes, 1, 296-311.
16. Donahue, R. R., Kessler, S. S., Bodine, D., McDonagh, K., Dunbar. C., Goodman, S., Agricola, B., Byrne, E., Raffeld, M. and Moen, R. (1992) Helper virus induced T cell lymphoma in nonhuman primates after retroviral mediated gene transfer. J. Exp. Med., 176, 1125-1135.
17. Urnovitz, H. H. and Murphy, W. W. (1996) Human endogenous retroviruses: nature, occurrence, and clinical implications in human disease. Clin. Microbiol. Rev., 9, 72-99.
18. Kaminski, R., Chen, Y., Fischer, T., Tedaldi, E., Napoli, A., Zhang, Y., Kam, J., Hu, W. and Khalili. K. (2016) Elimination of HIV-1 Genomes from Human T-lymphoid Cells by CRISPR/Cas9 Gene Editing. Sci. Rep., 6, 22555.
19. Yang, L., Giell, M., Niu, D., George, H., Lesha, E., Grishin, D., Aach, J., Shrock, E., Xu, W., Poci, J., et al. (2015) Genome-wide inactivation of porcine endogenous retroviruses (PERVs). Science., 350, 1101-1104.
20. Niu, D., Wei, H.-J., Lin, L., George, H., Wang, T., Lee, I.-H., Zhao, H.-Y., Wang, Y., Kan, Y., Shrock. E., et al. (2017) Inactivation of porcine endogenous retrovirus in pigs using CRISPR-Cas9. Science., 357, 1303-1307.
21. Fu. Y., Foden, J. J., Khayter, C., Maeder, M. M., Reyon, D., Joung, J. K. and Sander, J. J. (2013) High-frequency off-target mutagenesis induced by CRISPR-Cas nucleases in human cells. Nat. Biotechnol., 31, 822-826.
22. Zhu, L. L., Holmes, B. B., Aronin, N. and Brodsky, M. M. (2014) CRISPRseek: A Bioconductor package to identify target-specific guide RNAs for CRISPR-Cas9 genome-editing systems. PLoS One, 9, e108424.
23. Xu, H., Xiao, T., Chen, C.-H., Li, W., Meyer, C. C., Wu, Q., Wu, D., Cong, L., Zhang, F., Liu, J. J., et al. (2015) Sequence determinants of improved CRISPR sgRNA design. Genome Res., 25, 1147-1157.
24. Chari, R., Mali, P., Moosbumer, M. and Church, G. G. (2015) Unraveling CRISPR-Cas9 genome engineering parameters via a library-on-library approach. Nat. Methods, 12, 823.
25. Sander, J. J., Zaback, P., Joung, J. J., Voytas, D. D. and Dobbs, D. (2007) Zinc Finger Targeter (ZiFiT): An engineered zinc finger/target site design tool. Nucleic Acids Res., 35, 599-605.
26. Sander, J. J., Maeder, M. M., Reyon, D., Voytas, D. D., Joung, J. J. and Dobbs, D. (2010) ZiFiT (Zinc Finger Targeter): An updated zinc finger engineering tool. Nucleic Acids Res., 38, 462-468.
27. Ran, F. F., Hsu, P. P., Wright. J., Agarwala, V., Scott, D. D. and Zhang, F. (2013) Genome engineering using the CRISPR-Cas9 system. Nat. Protoc., 8, 2281-2308.
28. Brinkman. E. K., Chen, T., Amendola, M. and Van Steensel, B. (2014) Easy quantitative assessment of genome editing by sequence trace decomposition. Nucleic Acids Res., 42, 1-8.
29. Yoon, H. and Leitner, T. (2015) PrimerDesign-M: A multiple-alignment based multiple-primer design tool for walking across variable genomes. Bioinformatics, 31, 1472-1474.
30. Fadrosh, D. W., Ma, B., Gajer, P., Sengamalay, N., Ott, S., Brotman, R. R. and Ravel, J. (2014) An improved dual-indexing approach for multiplexed 16S rRNA gene sequencing on the Illumina MiSeq platform. Microbiome, 2, 6.
31. Le Foum, V., Girod, P. P., Buceta, M., Regamey, A. and Mermod, N. (2014) CHO cell engineering to prevent polypeptide aggregation and improve therapeutic protein secretion. Metab. Eng., 21, 91-102.
32. Feschotte, C. and Gilbert, C. (2012) Endogenous viruses: insights into viral evolution and impact on host biology. Nat. Rev. Genet., 13, 283.
33. Morikawa, Y., Hinata, S., Tomoda, H., Goto, T., Nakai, M., Aizawa, C., Tanaka, H. and Mura, S. (1996) Complete Inhibition of Human Immunodeficiency Virus Gag Myristoylation Is Necessary for Inhibition of Particle Budding. J. Biol. Chem., 271, 2868-2873.
34. Wapling, J., Srivastava, S., Shehu-Xhilaga, M. and Tachedjian, G. (2007) Targeting Human Immunodeficiency Virus Type 1 Assembly, Maturation and Budding. Drug Target Insights, 2, 159-182.
35. Henzy, J. J., Gifford, R. R., Johnson, W. W. and Coffin, J. J. (2014) A Novel Recombinant Retrovirus in the Genomes of Modern Birds Combines Features of Avian and Mammalian Retroviruses. J. Virol., 88, 2398-2405.
36. Segura-Morales, C., Pescia, C., Chatellard-Causse, C., Sadoul, R., Bertrand, E. and Basyuk, E. (2005) Tsg101 and Alix Interact with Murine Leukemia Virus Gag and Cooperate with Nedd4 Ubiquitin Ligases during Budding. J. Biol. Chem., 280, 27004-27012.
37. Daer, R. R., Cutts, J. J., Brafman, D. D. and Haynes, K. K. (2017) The Impact of Chromatin Dynamics on Cas9-Mediated Genome Editing in Human Cells. ACS Synth. Biol., 6, 428-438.
38. Gosselin, K., Deruy, E., Martien, S., Vercamer, C., Bouali, F., Dujardin, T., Slomianny, C., Houel-Renault, L., Chelli, F., De Launoit, Y., et al. (2009) Senescent keratinocytes die by autophagic programmed cell death. Am. J. Pathol., 174, 423-435.
39. Lemos, B. B., Kaplan, A. A., Bae, J. J., Ferrazzoli, A. A., Kuo, J., Anand, R. R., Waterman, D. D. and Haber, J. J. (2018) CRISPR/Cas9 cleavages in budding yeast reveal templated insertions and strand-specific insertion/deletion profiles. Proc. Natl. Acad. Sci., 115, E2040 LP-E2047.
40. Bae, S., Kweon, J., Kim, H. H. and Kim, J. (2014) Microhomology-based choice of Cas9 nuclease target sites. Nat. Methods, 11, 705-706.
41. Kostyrko, K., Neuenschwander, S., Junier, T., Regamey, A., Iseli, C., Schmid-Siegert, E., Bosshard, S., Majocchi, S., Le Fourn, V., Girod, P.-A., et al. (2017) MAR-Mediated transgene integration into permissive chromatin and increased expression by recombination pathway engineering. Biotechnol. Bioeng., 114, 384-396.
42. Kostyrko, K. and Mermod, N. (2015) Assays for DNA double-strand break repair by microhomology-based end-joining repair mechanisms. Nucleic Acids Res., 44, e56.
43. Sfeir, A. and Symington, L. L. (2015) Microhomology-Mediated End Joining: A Back-up Survival Mechanism or Dedicated Pathway?Trends Biochem. Sci., 40, 701-714.
44. Yu, A. A. and McVey, M. (2010) Synthesis-dependent microhomology-mediated end joining accounts for multiple types of repair junctions. Nucleic Acids Res., 38, 5706-17.
45. Lin, Y., Cradick, T. T., Brown, M. M., Deshmukh, H., Ranjan, P., Sarode, N., Wile, B. B., Vertino, P. P., Stewart, F. F. and Bao, G. (2014) CRISPR/Cas9 systems have off-target activity with insertions or deletions between target DNA and guide RNA sequences. Nucleic Acids Res., 42, 7473-7485.
46. Byrne, G., O'Rourke, S. S., Alexander, D. D., Yu, B., Doran, R. R., Wright, M., Chen, Q., Azadi, P. and Berman, P. P. (2018) CRISPR/Cas9 gene editing for the creation of an MGAT1-deficient CHO cell line to control HIV-1 vaccine glycosylation. PLoS Biol., 16, 1-23.
47. Semaan, M., Ivanusic, D. and Denner, J. (2015) Cytotoxic effects during knock out of multiple Porcine Endogenous Retrovirus (PERV) sequences in the pig genome by Zinc Finger Nucleases (ZFN). PLoS One, 10, 1-18.
48. Wang, T., Birsoy, K., Hughes, N. W., Krupczak, K. M., Post, Y., Wei, J. J., Lander, E. E. and Sabatini, D. D. (2015) Identification and characterization of essential genes in the human genome. Science., 350, 1096-1101.
49. Aguirre, A. A., Meyers, R. R., Weir, B. B., Vazquez, F., Zhang, C. C., Ben-David, U., Cook, A., Ha, G., Harrington, W. F., Doshi, M. M., et al. (2016) Genomic copy number dictates a gene-independent cell response to CRISPR/Cas9 targeting. Cancer Discov., 6, 914-929.
50. Ihry, R. R., Worringer, K. K., Salick, M. M., Frias, E., Ho, D., Theriault, K., Kommineni, S., Chen, J., Sondey, M., Ye, C., et al. (2018) P53 inhibits CRISPR-Cas9 engineering in human pluripotent stem cells. Nat. Med., 24, 939-946.
51. O'Connor, M. J. (2015) Targeting the DNA Damage Response in Cancer. Mol. Cell. 60, 547-560.
52. Ordando, S. S., Santiago, Y., DeKelver, R. R., Freyvert, Y., Boydston, E. E., Moehle, E. E., Choi, V. V., Gopalan, S. S., Lou, J. J., Li, J., et al. (2010) Zinc-finger nuclease-driven targeted integration into mammalian genomes using donors with limited chromosomal homology. Nucleic Acids Res., 38, 1-15.
53. Cristea, S., Freyvert, Y., Santiago, Y., Holmes, M. M., Umov, F. F., Gregory, P. P. and Cost, G. G. (2013) In vivo cleavage of transgene donors promotes nuclease-mediated targeted integration. Biotechnol. Bioeng., 110, 871-880.
54. Guirouilh-Barbat, Lambert, S., Bertrand, P. and Lopez, B. B. (2014) Is homologous recombination really an error-free process?Front. Genet., 5, 175.
55. Benson, F. F., Stasiak, A. and West, S. S. (1994) Purification and characterization of the human Rad51 protein, an analogue of E. coli RecA. EMBO J., 13, 5764-71.
56. Stark, J. J., Hu, P., Pierce, A. A., Moynahan, M. M., Ellis, N. and Jasin, M. (2002) ATP hydrolysis by mammalian RAD51 has a key role during homology-directed DNA repair. J. Biol. Chem., 277, 20185-20194.
57. Lee, J. J., Park, J. J., Ha, T. T., Samoudi, M., Lewis, N. N., Palsson, B. B., Kildegaard, H. H. and Lee, G. G. (2018) Revealing Key Determinants of Clonal Variation in Transgene Expression in Recombinant CHO Cells Using Targeted Genome Editing. ACS Synth. Biol., 10.1021/acssynbio.8b00290.
58. Pilbrough, W., Munro, T. T. and Gray, P. (2009) Intraclonal protein expression heterogeneity in recombinant CHO cells. PLoS One, 4, e8432.
59. Sigal, A., Milo, R., Cohen. A., Geva-Zatorsky. N., Klein, Y., Liron, Y., Rosenfeld, N., Danon, T., Perzov, N. and Alon, U. (2006) Variability and memory of protein levels in human cells. Nature, 444, 643-646.
60. Schmieder, V., Bydlinski, N., Strasser, R., Baumann, M., Kildegaard, H. H., Jadhav, V. and Borth, N. (2018) Enhanced Genome Editing Tools For Multi-Gene Deletion Knock-Out Approaches Using Paired CRISPR sgRNAs in CHO Cells. Biotechnol. J., 13, 1700211.
61. Hu, W., Kaminski, R., Yang, F., Zhang, Y., Cosentino, L., Li, F., Luo, B., Alvarez-Carbonell, D., Garcia-Mesa, Y., Kam, J., et al. (2014) RNA-directed gene editing specifically eradicates latent and prevents new HIV-1 infection. Proc. Natl. Acad. Sci., 111, 11461-11466.
62. Manrique, M. M., Celma, C. C. P., Gonzdlez, S. S. and Affranchino, J. J. (2001) Mutational analysis of the feline immunodeficiency virus matrix protein. Virus Res., 76, 103-113.
63. Kawada, S., Goto, T., Haraguchi, H., Ono, A. and Morikawa, Y. (2008) Dominant Negative Inhibition of Human Immunodeficiency Virus Particle Production by the Nonmyristoylated Form of Gag. J. Virol., 82, 4384-4399.
64. Nakamura, T., Yamada, K. K., Tomii, K. and Katoh, K. (2018) Parallelization of MAFFT for large-scale multiplesequence alignments. Bioinformatics, 34, 2490-2492.
65. Puri, V., Konda, S., Ranjit, S., Aouadi, M., Chawla, A., Chouinard, M., Chakladar, A. and Czech, M. M. (2007) Fat-specific Protein 27, a Novel Lipid Droplet Protein That Enhances Triglyceride Storage. J. Biol. Chem., 282, 34213-34218.
66. Boztug, K., Järvinen, P. P., Salzer, E., Racek, T., Mönch, S., Garncarz, W., Gertz, E. E., Schäffer, A. A., Antonopoulos, A., Haslam, S. S., et al. (2014) JAGN1 deficiency causes aberrant myeloid cell homeostasis and congenital neutropenia. Nat. Genet., 46, 1021-1027.

1.-8. (canceled)

9. An engineered cell comprising:
a genome of the cell comprising:
a sequence comprising a gag gene, an env gene, a pol gene and long terminal repeats (LTR) and comprising at least one alteration in the gag gene, env gene, pol gene and/or the LTRs, wherein the sequence is selected from:
(i) SEQ ID NO: 3,
(ii) SEQ ID NO: 1,
(iii) a variant of (i) or (ii); or
(iv) a sequence having more than 95%, 96%, 97%, 98%, 99% sequence identity with (i) and/or (ii) outside the gag, env, pol gene and/or the LTRs,
said at least one alteration being selected from the group consisting of insertions, deletions, substitutions and combinations thereof.

10. The engineered cell of claim 9, wherein the at least one alteration is in the gag, env, pol gene and/or the LTRs is in not more than 100, 90, 80, 70, 60, 50, 40, 30, 20, 15, 10, 5, 4, 3, 2 nucleotides including consecutive nucleotides, or 1 nucleotide of the gag, env, pol gene and/or the LTRs.

11. The engineered cell of claim 9, wherein the genome comprises:
(i) not more than 10%, 20%, 30%, 40%, 50% consecutive nucleotides of SEQ ID NO: 3, or
(ii) a sequence having more than 90% sequence identity with (i).

12. The engineered cell of claim 9, wherein the alteration(s) in the at least one full-length group 1 type-C ERV sequence(s) is in the gag gene, that comprises a PPYP motif and wherein (i) sequences encoding the PPYP motif and/or a sequence up to 5, 10, 15, 20, 25, 30, 35, 40, 45 or 50 nucleotides, including consecutive nucleotides, 5′ and/or 3′ flanking the sequences in (i) comprise the alteration(s).

13. The engineered cell of claim 9, wherein the genome comprises not more than 15, 14, 13, 12, 11, 10, 9, 8, 7, 6, 5, 4, 3, 2, 1 alteration(s) in the group 1 type-C ERV sequences or not more than 15, 14, 13, 12, 11, 10, 9, 8, 7, 6, 5, 4, 3, 2, 1 altered group 1 type-C ERV sequences.

14. The engineered cell of claim 9, wherein the alteration(s) is/are deletions, insertions, substitutions or combinations thereof, preferably alterations of the N-terminal Myr motif-encoding DNA sequence, such as one or several mutations that inhibits the myristoylation of the GAG protein by removing or substituting the amino-terminal glycine residue, or a PPYP mutation that inhibits the release of viral particles from the host cell, or one or several frameshift mutations that infer with a translation of the gag mRNA into a full-length GAG protein.

15. The engineered cell of claim 9, wherein the alteration(s) is/are frameshift mutation(s).

16. An engineered cell comprising:
a genome of the cell comprising group 1 type-C ERV sequences integrated into the genome, wherein at least one, including a singular, full-length group 1 type-C ERV sequence, such as SEQ ID NO: 3 or at least 10%, 20%, 30%, 40%, 50%, 60%, 70% 80% 90% or 100% consecutive nucleotides of SEQ ID NO: 3 and optionally 5′ and/or 3′ flanking regions of SEQ ID NO: 3, including 1-50, 30-100, 50-150, 100-200 or more than 200, 300, 400 or more than 500 consecutive nucleotides flanking SEQ ID NO: 3 are deleted from the genome.

17. The engineered cell of claim 16, wherein the flanking regions are SEQ ID NO: 4 and SEQ ID NO:5, respectively.

18. The engineered cell of claim 16, wherein the genome of the cell comprises:
(i) at least 80%, 90%, 95%, 98%, 99% or 100% consecutive nucleotides of SEQ ID NO: 4 and/or sequences having at least 90%, 95%, 98% or 99% sequence identity therewith and, directly adjacent thereto,
(ii) at least 80%, 90%, 95%, 98%, 99% or 100% consecutive nucleotides of SEQ ID NO: 5 and/or sequences having at least 90%, 95%, 98% or 99% sequence identity therewith.

19. The engineered cell of claim 16, wherein the alteration(s) are insertions of at least 5, 10, 15, 20, 25, 30, 50 or 100 nucleotides, deletions of at least 5, 10, 15, 20, 25, 30, 50 or 100 nucleotides, including consecutive nucleotides, or combinations thereof or combinations of insertions, substitution and/or deletions resulting together in an addition and/or removal of at least 5, 10, 15, 20, 25, 30, 50 or 100 nucleotides.

20. (canceled)

21. The engineered cell of claim 16, wherein the cell releases a number of viral particles (VP), viral like particles (VLP) or retroviral (like) particles (RV(L)Ps) per unit of time, the number being reduced, preferably more than 2-fold, more preferably more than 10-fold, even more preferably more than 50-fold, more than 100-fold, more than 150-fold, more than 200-fold or more than 250-fold relative to the VPs, VLPs or RV(L)Ps per unit of time released by its non-engineered counterpart.

22. The engineered cell of claim 16, wherein said engineered cell releases no or substantially no VP, in particular RVP.

23. The engineered cell of claim 16, wherein said cell further comprises a transgene, preferably integrated into the genome.

24. The engineered cell of claim 23, wherein the transgene is a marker gene encoding a marker protein such as GFP (green fluorescent protein) and/or a biotherapeutic and/or a non-coding RNA.

25. An engineered cell comprising:
a genome of the cell comprising SEQ ID NO: 3 or a variant thereof, and further comprising a sequence encoding a siRNA, wherein a target sequence of the siRNA is located within SEQ ID NO:3 or the variant thereof, more preferably within a sequence of SEQ ID NO: 3 encoding the Gag precursor protein or a variant thereof.

26. A method for producing a transgene product comprising:
providing the engineered cell of claim 9, introducing at least one transgene encoding the transgene product, such as a biotherapeutic, into the engineered cell, and
expressing the at least one transgene in the cell, wherein said engineered cell releases no or substantially no VP or VLP.

27. A detection kit and its use comprising:
(i) at least one primer against SEQ ID NO: 3, and/or
(ii) at least one primer against SEQ ID NO: 4 or 5, and
instructions how to use the primers of (i) and/or (ii) to detect the presence or absence of SEQ ID NO: 1, of SEQ ID NO: 3 from a genome of a CHO cell or a mutation within SEQ ID NO: 3 of the genome of the CHO cell.

28. The engineered cell of claim 16, wherein the engineered cell is of a mammalian cell line.

29. The engineered cell of claim 16, wherein the engineered cell is an engineered CHO cell.
